(19) United States
(12) Patent Application Publication
(10) Pub. No.: US 2025/0179576 A1
(43) Pub. Date: Jun. 5, 2025

(54) MICROBIAL SIGNATURES OF AUTISM SPECTRUM DISORDER

(71) Applicant: Piton Therapeutics, Inc., Watertown, MA (US)

(72) Inventors: Robert Hardie Mills, Allston, MA (US); Eric Hou-Jen Wang, La Jolla, CA (US); Aries Chavira, Allston, MA (US)

(73) Assignee: Piton Therapeutics, Inc., Watertown, MA (US)

(21) Appl. No.: 18/838,675

(22) PCT Filed: Feb. 17, 2023
(86) PCT No.: PCT/US2023/062882

§ 371 Date: Aug. 15, 2024

Related U.S. Application Data
(60) Provisional application No. 63/311,757, filed on Feb. 18, 2022.

(51) Int. Cl. C12Q 1/6883; A61K 31/194; A61K 38/51; A61K 45/06; C12Q 1/6809; C12Q 1/6851; C12Q 1/6869; C12Q 1/689; G06N 20/00; G16H 20/10; G16H 50/30

(52) U.S. Cl. CPC C12Q 1/6883; A61K 31/194; A61K 38/51; A61K 45/06; C12Q 1/6809; C12Q 1/6851; C12Q 1/6869; C12Q 1/689; C12Y 401/0304; G06N 20/00; G16H 20/10; G16H 50/30; C12Q 2600/158

(57) ABSTRACT

Aspects of the disclosure relate to methods and compositions for diagnosing and/or treating subjects having one or more symptoms of Autism Spectrum Disorder (ASD) or subjects at risk of developing ASD. In some embodiments, methods comprise determining and/or modulating levels of chorismate and/or a molecule within a chorismate metabolic pathway in a subject. In some embodiments, methods comprise determining and/or modulating levels of a microbial feature (e.g., a Sarcina bacterium) in a subject. In some embodiments, methods comprise determining and/or modulating levels of proton flux in a gastrointestinal tract in a subject.

### RELATED APPLICATIONS

This application claims the benefit under 35 U.S.C. § 119(e) of U.S. Provisional Application No. 63/311,757, filed Feb. 18, 2022, and entitled “Microbial Signatures of Autism Spectrum Disorder,” which is hereby incorporated by reference in its entirety for all purposes.

### BACKGROUND

Autism Spectrum Disorder (ASD) represents a group of neurodevelopmental disorders characterized by a spectrum of behavioral, speech, and social impairments. Recent reports suggest that diagnoses have increased to 1 in 44 children (2.3%) in the United States, highlighting ASD as a disorder of increasing prevalence. The precise etiology of autism is unknown but is thought to be multifactorial.

The human gut microbiota has emerged as an important factor in a wide range of diseases, including neurological conditions such as ASD. Mechanistically, there are multiple hypotheses for how microbes may be involved in ASD, including the maternal immune activation model, the production of neurotransmitters such as serotonin or GABA, the modulation of vagus nerve signaling, and the production of molecules such as 4-ethylphenylsulfate or short-chain fatty acids. However, it remains unclear which—if any—of these mechanisms drives the development of ASD. Accordingly, improved methods and compositions for diagnosing and/or treating ASD are needed.

### SUMMARY OF INVENTION

In some embodiments, aspects of the invention relate to systems and methods for identifying one or more molecules and/or microbial features associated with Autism Spectrum Disorder (ASD) in a subject. In some embodiments, aspects of the invention relate to methods and compositions for treating one or more symptoms of ASD and/or comorbidities of ASD in a subject or for reducing the risk of the subject developing ASD.

Systematic meta-analyses of studies that profiled the gut microbiome of individuals with ASD have revealed, in some aspects, that commonly differentially abundant metabolic pathways represented by the gut microbiome of individuals with ASD were related to a common precursor molecule, chorismate.

Accordingly, in some embodiments, methods and compositions for treating ASD comprise modulating molecules within chorismate-related pathways (e.g., chorismate metabolic pathways). In some embodiments, a method of treating a subject having one or more symptoms of ASD and/or of comorbidities of ASD comprises modulating chorismate levels in the subject. In some embodiments, a method of treating a subject having one or more symptoms of ASD and/or of comorbidities of ASD comprises modulating levels of one or more molecules in a chorismate metabolic pathway in the subject. In some embodiments, a method of treating a subject having one or more symptoms of ASD and/or of comorbidities of ASD comprises modulating the activity or level of one or more enzymes or metabolites in the chorismate metabolic pathway (e.g., one or more of the non-limiting examples of molecules listed in Table 1 or Table 2). In some embodiments, modulating comprises increasing the level and/or activity of an enzyme (e.g., administering an agonist). In some embodiments, modulating comprises decreasing the level and/or activity of an enzyme (e.g., administering an antagonist). In some embodiments, chorismate mutase is modulated. In some embodiments, chorismate lyase is modulated (e.g., inhibited). In some embodiments, isochorismate synthase is modulated (e.g., inhibited). In some embodiments, aminodeoxychorismate synthase (also known as PABA synthase) is modulated (e.g., inhibited). In some embodiments, anthranilate synthase is modulated.

In some embodiments, a modulating agent (e.g., an agonist or antagonist of one or more enzymes or other molecules in the chorismate metabolic pathway) is administered to a subject having one or more symptoms or ASD and/or of comorbidities of ASD or to a subject at risk of developing autism.

In some embodiments, the level of chorismate or one or more molecules within chorismate-related pathways (e.g., chorismate metabolic pathways) is modulated in the gut of a subject (e.g., within the microbiota of a subject). In some embodiments, the level of chorismate or one or more molecules within chorismate-related pathways (e.g., chorismate metabolic pathways) is modulated in the blood or a tissue (e.g., brain, colon, and/or small intestine) of a subject.

In some embodiments, the level of chorismate or one or more molecules within chorismate-related pathways (e.g., chorismate metabolic pathways) is modulated by administering a composition that modulates expression and/or activity of one or more enzymes in the chorismate metabolic pathway (e.g., within microbes in the gut of the subject or within the blood or tissue of the subject). In some embodiments, the composition comprises one or more agonists and/or antagonists of one or more enzymes in the chorismate metabolic pathway.

In some embodiments, the level of chorismate or one or more molecules within chorismate-related pathways (e.g., chorismate metabolic pathways) is modulated by administering a composition that modulates levels of microbial organisms within the gut of the subject. In certain embodiments, the microbial organisms produce chorismate and/or one or more chorismate metabolites. In some embodiments, the composition is a microbial composition. In some embodiments, the composition comprises one or more antibiotics, probiotics, and/or prebiotics that can modulate the relative amount of different microbial organisms in the gut of a subject.

In some embodiments, methods and compositions for assisting in the diagnosis of ASD in a subject having one or more symptoms of ASD and/or of comorbidities of ASD or at risk of developing ASD comprise determining a level of chorismate and/or one or more molecules within a chorismate metabolic pathway in a sample obtained from the subject. In some embodiments, a subject is identified as having ASD, or being at risk for ASD, if the level of one or more of such molecules is statistically correlated with ASD, for example based on the analysis of data from subjects diagnosed as having ASD.

In some embodiments, a sample is a fecal sample. In some embodiments, a sample is a serum sample. In some embodiments, a sample is a salivary, buccal, nasal, urine, cerebrospinal fluid, or gastro-intestinal sample (e.g., swab or biopsy).

In some embodiments, a subject is a human. In some embodiments, a subject is a child. In some embodiments, a subject is an adult. In some embodiments, a subject is at risk of developing ASD and/or one or more comorbidities of ASD. In some embodiments, a subject is diagnosed as having one or more signs or symptoms of ASD and/or one or more comorbidities of ASD.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 shows a schematic illustration of the shikimate pathway;

FIG. 2 shows a schematic illustration of chorismate metabolic pathways;

FIG. 3 shows, according to some embodiments, association of ASD status to microbiome alpha and beta-diversity. FIG. 3A shows Principle Coordinates Analysis (PCoA) from the unweighted UniFrac distance matrix of 1740 samples. FIG. 3B shows PcoA from the weighted UniFrac distance matrix of 1740 samples. FIG. 3C shows Pseudo-F statistics from a PERMANOVA beta-group significance test of ASD vs. control from unweighted and weighted UniFrac distance metrics. Bars with full color opacity refer to PERMANOVA tests with a P<0.05. FIG. 3D shows Log 2 fold change in alpha-diversity values of ASD to controls for each cohort and sized by significance. Boxplots show the median, quartiles, and 1.5× inter-quartile range of the data distribution. P-values were generated from unadjusted two-tailed t-tests.

FIG. 4 shows, according to some embodiments, taxonomic and functional differences between ASD and controls. FIG. 4A shows Log 2 fold change in the relative abundance of ASD/controls of the most significant phyla. Individual points are colored by cohort, with size indicating a significant difference between ASD and controls (unadjusted two-tailed t-tests p-value<0.05). FIG. 4B shows Log 2 fold change in the relative abundance of ASD/controls of the most significant genera, with the same coloring and sizing of individual points as FIG. 4A. FIGS. 4C-4E show per-sample plots of the log 10 transformed relative abundance of each sample for various classes and genera, stratified by ASD status. Boxplots show the median, quartile, and 1.5× inter-quartile range of the data distribution. FIG. 4C shows relative abundance of the class Actinobacteria in ASD vs. control children. FIG. 4D shows relative abundance of the genus Prevotella in ASD vs. control children. FIG. 4E shows relative abundance of the genus Bifidobacterium in ASD vs. control children. FIG. 4F shows significant microbial pathways when comparing ASD and controls. Plotted are the log 2 fold changes of the normalized pathway abundance values in ASD/control for the most commonly significant bacterial metabolic pathways for each cohort processed individual. Hierarchical clustering of studies and pathways was performed using Euclidean distance. Points are colored by log 2 fold change and sized by significance (unadjusted two-tailed t-tests P<0.05). FIG. 4G shows normalized log 2 fold change values of ASD/control for every pathway involving menaquinones and ubiquinones in each cohort processed individually or in aggregate. Individual points are colored by Log 2 ASD/control fold-change and sized to indicate statistical significance between ASD and controls (unadjusted two-tailed t-tests of unequal variance p-value <0.05). FIG. 4H shows normalized log 2 fold change values of ASD/control for chorismate, aromatic amino acids, and vitamin B pathways from each cohort processed individually or in aggregate. Individual points are colored by Log 2 ASD/control fold-change and sized to indicate statistical significance between ASD and controls (unadjusted two-tailed t-tests of unequal variance p-value <0.05). The L-tyrosine, superpathway of L-tryptophan biosynthesis, L-tryptophan biosynthesis, superpathway of L-tyrosine biosynthesis, and superpathway of L-phenylalanine biosynthesis pathways are related to aromatic amino acids. The adenosylcobalamin biosynthesis and superpathway of tetrahydrolate biosynthesis are vitamin B related pathways.

FIG. 5 shows, according to some embodiments, evaluations of machine learning performance with respect to ASD status prediction. FIG. 5A shows bar plots of the mean AUC±SEM from 50 iterations. Eleven classification algorithms were tested using the aggregated ASV-level data set. The Gradient Boosting Classifier and AdaBoost models performed best and were further tested. FIG. 5B shows the mean accuracy of the gradient boosting classifier models trained and tested on both the aggregated data and on each study individually on read counts collapsed at different taxonomic levels. Error bars show the standard error from 5-fold cross validation repeated 10 times. FIG. 5C shows the relative abundance and SHAP value for each sample of the top 25 features plotted in decreasing feature importance from the gradient boosting classifier model trained on the aggregate data at the ASV level. FIG. 5D is a cluster heatmap generated and colored by the SHAP values of the top 25 features from the gradient boosting classifier model trained and tested on the aggregate data and each cohort independently. Clustering of both cohorts and features was performed using hierarchical clustering of Euclidean distances.

FIG. 6, shows, according to some embodiments, evaluation of study design factors that influence the performance of machine learning models and taxonomic abundance. FIGS. 6A-6D show (top) per-study log 2 fold change of ASD to controls of the genus Prevotella (FIG. 6A), ratio of Prevotella to Bacteroidetes (FIG. 6B), order Desulfovibrionales (FIG. 6C), and class Deltaproteobacteria (FIG. 6D) of data sequenced from the V4 and V3-V4 hypervariable regions respectively and (bottom) log 10 transformed relative abundance of the genus Prevotella (FIG. 6A), ratio of Prevotella to Bacteroidetes (FIG. 6B), order Desulfovibrionales (FIG. 6C), and class Deltaproteobacteria (FIG. 6D) in each sample from the V4 and V3-V4 regions, split by ASD status (V4 n=1024, V3-V4 n=352). P-values were calculated by unadjusted Wilcoxon two-tailed t-tests. Boxplots show the median quartiles and 1.5× inter-quartile range of the data distribution. FIG. 6E shows machine learning AUC, accuracy, and F1 score of the gradient boosting clarifier model trained to predict ASD status from samples split by sequencing depth and variable region (sequencing depth: >140000 n=851, >6000 n=640, variable region: V3-V4 n=356, V4 n=1032, V1-V2 n=103). FIG. 6F shows AUC, accuracy, and F1 score of the gradient boosting clarifier model at predicting ASD status from samples split by control relationship, country, and sex (control relationship: related controls n=462, unrelated controls n=1029; country: USA n=761, Ecuador n=46, China n=684; sex: unknown n=168, male n=1021, female n=302). Bar plots show mean±SEM. FIG. 6G shows a heat map of the GBC model's average AUC trained and tested on samples binned at each potential age range from 1-16.

FIG. 7 shows, according to some embodiments, chorismate branchpoint enzyme abundance. FIG. 7A shows per-study log 2 fold change in the normalized enzyme number abundance of ASD/control samples from the five main chorismate branchpoint enzymes. FIG. 7B shows boxplots of the predicted and normalized chorismate lyase EC number by sample, stratified by cohort and ASD status. A Wilcoxon two-tailed t-test was performed between groups, with significant differences indicated to the right of each cohort (*, p<0.05; **, p<0.01, *** p<0.001). Boxplots show the median, quartiles and 1.5× inter-quartile range of the data distribution. FIG. 7C shows boxplots of the predicted and normalized menF gene abundance per sample and stratified by cohort and ASD status. A Wilcoxon two-tailed t-test was performed between groups (*, p<0.05; **, p<0.01, *** p<0.001). FIG. 7D shows a heatmap depicting the presence or absence of bacterial features containing the menF gene, highlighting the bacterial features present in the greatest number of studies. The heatmap indicates the bacteria's presence (1) or absence (0) in each cohort.

FIG. 8A shows, according to some embodiments, a plot of Log 2 fold change between ASD and control subjects for commonly altered genera.

FIG. 8B shows, according to some embodiments, histograms and KDE curves showing distribution of compositional abundance of Sarcina in the gut microbiome of ASD and control subjects.

FIG. 8C shows, according to some embodiments, a heatmap describing the fractional abundance of Sarcina in ASD or control subjects, divided by cohorts and study factors.

FIG. 9A shows, according to some embodiments, a bubble plot showing commonly significant pathways found in the gut microbiome of ASD subjects compared to controls.

FIG. 9B shows, according to some embodiments, histograms and KDE curves showing distribution of compositional abundance of the superpathway of chorismate metabolism in the gut microbiome of ASD and control subjects.

FIG. 9C shows, according to some embodiments, a heatmap describing the fractional abundance of superpathway of chorismate metabolism in ASD or control subjects, divided by cohorts and study factors.

FIG. 9D shows, according to some embodiments, Venn diagrams describing the overlap between superpathway of chorismate metabolism and various features linked to ASD.

FIG. 10A shows, according to some embodiments, a bubble plot showing commonly significant metabolic influx differences found in the gut microbiome of ASD subjects compared to controls.

FIG. 10B shows, according to some embodiments, histograms and KDE curves showing distribution of the influx of protons by the gut microbiome of ASD and control subjects.

FIG. 10C shows, according to some embodiments, a heatmap describing the fractional abundance of ASD or control subjects containing more than a threshold influx of protons by the gut microbiome, divided by cohorts and study factors.

FIG. 11 shows, according to some embodiments, an UpSet plot cross-comparing the patient populations of three subtypes of ASD.

FIG. 12A shows, according to some embodiments, a bar plot of the top 30 metabolites altered among ASD subjects with increased chorismate metabolism genes when given increased chorismate.

FIG. 12B shows, according to some embodiments, boxplots showing the influx difference for L-tyrosine for ASD subjects of the chorismate subtype when given increased chorismate in a simulated diet.

FIG. 12C shows, according to some embodiments, boxplots showing the influx difference for folate for ASD subjects of the chorismate subtype when given increased chorismate in a simulated diet.

FIG. 12D shows, according to some embodiments, boxplots showing the influx difference for indole for ASD subjects of the chorismate subtype when given increased chorismate in a simulated diet.

FIG. 12E shows, according to some embodiments, boxplots showing the influx difference for menaquinone 8 for ASD subjects of the chorismate subtype when given increased chorismate in a simulated diet.

### DETAILED DESCRIPTION

Aspects of the invention relate to methods and compositions for diagnosing and/or treating subjects having one or more symptoms of Autism Spectrum Disorder (ASD) and/or subjects at risk of developing ASD. In some embodiments, compositions that modulate chorismate levels in a subject are useful to treat ASD. In some embodiments, compositions that modulate chorismate metabolism (e.g., the biosynthesis of molecules downstream of chorismate) in a subject are useful to treat ASD. In some embodiments, compositions that modulate the relative abundance of microbial organisms (e.g., Sarcina bacteria, Clostridium ventriculi) in a subject (e.g., in the gut of a subject) are useful to treat ASD. In some embodiments, compositions that modulate levels of proton flux in a gastrointestinal (GI) tract of a subject are useful to treat ASD.

According to aspects of the invention, evaluating the abundance of one or more genes within the superpathway of chorismate metabolism within the human gut microbiota can help determine the utility of administering one or more therapeutic interventions for one or more of the core symptoms of autism. In some embodiments, a therapeutic intervention can include a single therapeutic intervention or a combination of two or more therapeutic interventions.

In some embodiments, the diagnosis of ASD in a subject is aided by evaluating (e.g., determining and/or monitoring) the abundance of one or more genes (e.g., all of the genes or a subset of the genes) within the microbiota (e.g., gut microbiota) within the superpathway of chorismate metabolism in a sample from a subject. In some embodiments, the abundance of a gene can be evaluated by determining DNA and/or RNA levels for the gene (e.g., using DNA and/or RNA sequence data). In some embodiments, the abundance of a gene can be evaluated by determining the level of one or more gene products in a sample.

In some embodiments, the diagnosis of ASD in a subject is aided by evaluating (e.g., determining and/or monitoring) the levels of one or more metabolites of the superpathway of chorismate metabolism (e.g., a combination of such metabolites) in a sample from a subject.

In some embodiments, a method of treating a subject at risk for ASD or a subject having one or more symptoms of ASD and/or of comorbidities of ASD comprises administering a probiotic and/or a prebiotic to the subject, for example to modulate the abundance of a microbial organism containing one or more of the genes within the superpathway of chorismate metabolism. Accordingly, in some embodiments a probiotic and/or a prebiotic can be used as a therapeutic intervention, or in combination with one or more additional therapeutic interventions, to treat one or more of the core symptoms of autism.

In some embodiments, the administration of molecules that increase or decrease the activity of one or a combination of the chorismate branchpoint enzymes within the gut microbiota may be used as a therapeutic intervention for one or more of the core symptoms of autism.

In some embodiments, the administration of probiotics containing chorismate branchpoint enzymes with modulated activity levels may be used as a therapeutic intervention for one or more of the core symptoms of autism.

In some embodiments, the administration of molecules such as chorismate, chorismic acid, and/or a salt or ester of either thereof may be used as a therapeutic intervention for one or more of the core symptoms of autism. In some embodiments, the administration of other molecules within the superpathway of chorismate metabolism may be used as a therapeutic intervention for one or more of the core symptoms of autism. In some embodiments, the administration of a combination of chorismate, chorismic acid, and/or a salt or ester of either thereof and one or more other molecules within the superpathway of chorismate metabolism may be used as a therapeutic intervention for one or more of the core symptoms of autism.

In some embodiments, evaluating (e.g., determining and/or monitoring) the activity of chorismate branchpoint enzymes within the gut microbiota of a subject may be used as an aid in the diagnosis of ASD in the subject.

In some embodiments, evaluating (e.g., determining and/or monitoring) the activity of chorismate branchpoint enzymes within the gut microbiota of a subject may be used as an aid in the determining of the appropriate therapeutic intervention or combination of therapeutic interventions to administer to a subject.

These and other aspects of the invention are described in more detail below.

As used herein, “ASD” refers to Autism Spectrum Disorder.

As used herein, “core symptoms of autism” include, but are not limited to, irritability, sleeplessness, speech and communication issues, aggression, digestive issues, socialization issues, limited repetitive patterns of behavior and interests, anxiety attacks, self-aggression, and mood disorders.

As used herein, “superpathway of chorismate metabolism” refers to the pathway for the production of chorismate from D-erythrose 4-phosphate and phosphoenolpyruvate (i.e., the shikimate pathway) and the pathways for the conversion of chorismate to downstream metabolites (e.g., aromatic amino acids and their derivatives, ubiquinols, menaquinols, tetrahydrofolate, enterobactin). As used herein, a “chorismate metabolic pathway” refers to the superpathway of chorismate metabolism or a pathway within the superpathway of chorismate metabolism.

As used herein, genes in the superpathway of chorismate metabolism include, but are not limited to, the genes within any microorganism from the human gut microbiota involved in the production of chorismate from D-erythrose 4-phosphate and phosphoenolpyruvate and the conversion of chorismate into ubiquinols, menaquinols, tetrahydrofolate, enterobactin, L-tryptophan, L-tyrosine, L-phenylalanine, and the metabolic products of L-tryptophan, including but not limited to serotonin, melatonin, kyneurenine, indoles, and any intermediate molecules. A non-limiting list of gene names can be found in Table 1.

As used herein, metabolites of the superpathway of chorismate metabolism include, but are not limited to, the intermediate molecules involved in the production of chorismate from D-erythrose 4-phosphate and phosphoenolpyruvate or any of the molecules that are derived from chorismate. A non-limiting list of metabolite names can be found in Table 2.

As used herein, “chorismate branchpoint enzymes” include, but are not limited to, Chorismate mutase, Anthranilate Synthase, Aminodeoxychorismate synthase (also known as para-aminobenzoic acid (PABA) synthase), Isochorismate synthase, Menaquinone-specific isochorismate synthase, Chorismate dehydratase, and Chorismate lyase.

As used herein, “therapeutic interventions” include, but are not limited to, melatonin supplements, folate supplements, probiotics, fecal transplantations, vitamin K supplementation, tryptophan supplementation or any inventions modulating serotonin, kynurenine or indoles, fructo-oligosaccharides, shikimate or shikimic acid or any salt or ester thereof, chorismate or chorismic acid or any salt or ester thereof, and modulators (e.g., inhibitors or agonists) of chorismate branchpoint enzymes and/or one or more genes or proteins in Table 1 or Table 2.

Described herein are embodiments of methods and compositions for evaluating a subject's risk of having or developing autism spectrum disorder (ASD), for identifying a treatment to provide to a subject to reduce the risk of developing ASD or to mitigate or alleviate one or more symptoms of ASD and/or of comorbidities of ASD, and/or for treating the subject with the identified treatment.

According to some embodiments, methods described herein comprise obtaining a sample from a subject. In some embodiments, the subject is a human subject. In certain embodiments, the subject is male. In certain embodiments, the subject is female. The subject may be from any geographical location. In certain embodiments, the subject is from North America (e.g., the United States), Asia (e.g., China, South Korea), Europe (e.g., Italy), and/or South America (e.g., Ecuador).

The subject may be of any age. In certain embodiments, the subject is a child (e.g., 17 years old or younger). In certain embodiments, the subject is an adult (e.g., 18 years old or older). In some embodiments, the subject has an age in a range from 1-5 years old, 1-7 years old, 1-10 years old, 1-15 years old, 1-17 years old, 1-18 years old, 1-21 years old, 1-50 years old, 1-70 years old, 1-100 years old, 2-7 years old, 2-10 years old, 2-15 years old, 2-17 years old, 2-18 years old, 2-21 years old, 2-50 years old, 2-70 years old, 2-100 years old, 5-10 years old, 5-15 years old, 5-17 years old, 5-18 years old, 5-21 years old, 5-50 years old, 5-70 years old, 5-100 years old, 10-15 years old, 10-17 years old, 10-18 years old, 10-21 years old, 10-50 years old, 10-70 years old, 10-100 years old, 15-21 years old, 15-50 years old, 15-70 years old, 15-100 years old, 18-50 years old, 18-70 years old, 18-100 years old, 21-50 years old, 21-70 years old, 21-100 years old, 50-70 years old, 50-100 years old, or 70-100 years old.

In some embodiments, the sample is a fecal sample. In certain embodiments, obtaining the sample comprises collecting a fecal sample (e.g., collecting a stool sample, swabbing a rectal cavity). In some embodiments, the sample is a gastrointestinal sample. In certain cases, a gastrointestinal sample comprises a gastric aspirate, biopsy (e.g., mucosal biopsy, gastric tissue biopsy), intestinal fluid, endoscopic brush, and/or laser capture microdissection sample. In some embodiments, obtaining the sample from a subject comprises performing a biopsy, endoscopy, and/or colonoscopy on the subject. In some embodiments, the sample is a serum, cerebrospinal fluid (CSF), urine, buccal, nasal, or salivary sample. In some embodiments, obtaining the sample from a subject comprises collecting a serum sample, performing a spinal tap on the subject, collecting a urine sample, and/or swabbing a cheek, throat, and/or nasal cavity of the subject.

In some embodiments, methods described herein comprise analyzing a sample obtained from a subject to evaluate one or more characteristics of a microbiome of the subject. In some cases, the one or more characteristics comprise the identity of one or more microbes detected within the microbiome and/or the amounts or relative amounts of one or more microbes detected within the microbiome.

In some cases, the microbiome may be a microbiome of the subject's gastrointestinal tract (e.g., a gut microbiome that comprises gut microbiota). In some embodiments, the sample may represent characteristics of the subject's gastrointestinal tract. In some instances, the sample is a fecal sample. In some instances, the sample is a gastrointestinal sample. In certain cases, the gastrointestinal sample is taken from the gastrointestinal tract (e.g., the lower gastrointestinal tract, the upper gastrointestinal tract). The sample may be obtained during any suitable procedure, including but not limited to colonoscopy, endoscopy, swabbing, or brushing a part of the gastrointestinal tract.

While some examples described herein focus on the gastrointestinal microbiome (of the upper and/or lower gastrointestinal tract), embodiments are not so limited and other embodiments may relate to microbiomes for other parts of a subject's anatomy (e.g., sinuses, skin, lungs, oral mucosa, or other). Any of the previously-described techniques for obtaining samples may be used, when medically appropriate, for obtaining samples from these other portions of a subject's anatomy. Samples obtained may be prepared and then the microbes within them analyzed to determine microbial features. In some cases, embodiments may relate to microbiome products (e.g., chorismate and its metabolites) present in other parts of a subject's anatomy (e.g., blood, brain, etc.).

In some embodiments, methods described herein comprise determining a level (e.g., an abundance) of a molecule (e.g., chorismate, a molecule within a chorismate metabolic pathway) and/or a microbial feature (e.g., a microbial organism) in a sample from a subject.

In some embodiments, determining the level of the molecule and/or the microbial feature in comprises determining an abundance of one or more genes (e.g., one or more genes within the superpathway of chorismate metabolism, including but not limited to one or more genes listed in Table 1) present in a sample from the subject. In some embodiments, determining the abundance of one or more genes present in the sample comprises extracting DNA and/or RNA from at least a portion of the sample. Any nucleic acid extraction method known in the art may be used. In some embodiments, determining the abundance of one or more genes present in the sample further comprises amplifying at least a portion of the DNA and/or RNA to produce a plurality of amplicons. Any nucleic acid amplification method known in the art may be used. Non-limiting examples of suitable nucleic acid amplification methods include polymerase chain reaction (PCR) and isothermal amplification methods (e.g., loop-mediated isothermal amplification (LAMP), rolling circle amplification (RCA), nucleic acid sequence based amplification (NASBA)). In some embodiments, determining the abundance of one or more genes present in the sample comprises performing a quantitative nucleic acid amplification method. A non-limiting example of a suitable quantitative nucleic acid amplification method is quantitative PCR (qPCR). In some embodiments, determining the abundance of one or more genes present in the sample further comprises sequencing one or more amplicons. Any suitable nucleic acid sequencing method may be used. In certain embodiments, the nucleic acid sequencing method is a long-read sequencing method. In certain embodiments, the nucleic acid sequencing method is a short-read sequencing method. In some embodiments, the nucleic acid sequencing method is a next-generation sequencing method. In some embodiments, the nucleic acid sequencing method is performed using an Illumina, Pacific Biosciences, Oxford Nanopore, and/or Roche 454 sequencing platform.

In some embodiments, determining the abundance of one or more genes present in a sample comprises sequencing at least a portion of 16S ribosomal RNA (rRNA) of microbes present in the sample (e.g., performing 16S rRNA gene amplicon (16S) sequencing). In some cases, performing 16S sequencing comprises amplifying at least a portion of one or more regions of the 16S rRNA genome of microbes present in the sample to produce a plurality of amplicons. Any nucleic acid amplification method (e.g., PCR) may be used. In some embodiments, the one or more regions of the 16S rRNA genome comprise at least one hypervariable region (e.g., V4, V3-V4, V1-V2). In some cases, performing 16S sequencing further comprises sequencing one or more amplicons of the plurality of amplicons. Any suitable nucleic acid sequencing method may be used.

In some embodiments, determining the abundance of one or more genes present in a sample comprises performing shotgun metagenomic sequencing to sequence the metagenomic content of the sample. In some cases, shotgun metagenomic sequencing methods comprise extracting DNA from a sample, fragmenting the extracted DNA into DNA fragments, and sequencing the DNA fragments. Any suitable nucleic acid sequencing method may be used. In some cases, the resulting sequences of the DNA fragments may be analyzed to identify microbes present in the sample.

In some embodiments, determining the abundance of one or more genes present in a sample comprises determining the level of one or more gene products (e.g., enzymes) in a sample. The level of the one or more gene products may be determined according to any method known in the art, including but not limited to an enzyme-linked immunosorbent assay (ELISA) and other antibody-based assays, and liquid chromatography-mass spectrometry (LC-MS)-based proteomics and other protein-based quantification assays. In some embodiments, determining the abundance of one or more genes present in a sample comprises determining the level of one or more molecules (e.g., chorismate and/or its metabolites) that directly or indirectly interact with one or more gene products (e.g., enzymes) in a metabolic pathway (e.g., as a substrate or a product). The level of the one or more molecules (e.g., substrates, products) may be determined according to any method known in the art, including but not limited to high-performance liquid chromatography (HPLC), liquid chromatography-mass spectrometry (LC-MS), gas chromatography-mass spectrometry (GC-MS), and fluorescence-based assays.

In some embodiments, determining the level of the molecule and/or the microbial feature in a sample from a subject comprises measuring the level of the molecule and/or microbial feature in the sample. The level of the molecule and/or the microbial feature in the sample may be measured according to any method known in the art. Non-limiting examples of suitable methods include HPLC, LC-MS, GC-MS, and matrix-assisted laser desorption ionization time-of-flight mass spectroscopy (MALDI-TOF). In certain embodiments, the level of the microbial feature in the sample may be measured by plating at least a portion of the sample on different media and/or under different conditions to evaluate microbial growth and identify one or more microbial organisms present in the sample.

According to some embodiments, a method comprises determining, if the level of a molecule or a microbial feature is higher or lower than a threshold value, that the subject has at least one subtype of ASD or is at risk of developing at least one subtype of ASD. In some cases, the threshold value may be determined based on the level of the molecule or microbial feature in one or more neurotypical individuals (e.g., individuals who have not been diagnosed with ASD). In some cases, the threshold value may be determined by identifying an optimal discriminatory boundary between a first population of individuals diagnosed with ASD and a second population of neurotypical individuals.

According to some embodiments, a method comprises identifying a subtype of ASD that a subject has or is at risk of having. In some embodiments, the method comprises determining a composition of a fecal microbiome or a presence or level of one or more molecules and/or microbial features in a sample obtained from the subject. In some embodiments, the subtype of ASD is a chorismate subtype. In some cases, the chorismate subtype may be characterized by an altered level of chorismate and/or a molecule within a chorismate metabolic pathway (e.g., the shikimate pathway, the pathways for conversion of chorismate into aromatic amino acids and their derivatives, folate and its derivatives, ubiquinols, menaquinols, and enterobactin) in the sample (e.g., relative to one or more neurotypical individuals). In some cases, the altered level of chorismate and/or the molecule within the chorismate metabolic pathway may be above or below a certain threshold. In some embodiments, the subtype of ASD is a Sarcina subtype. In some cases, the Sarcina subtype may be characterized by an altered (e.g., elevated) level of a Sarcina bacterium in the sample (e.g., relative to one or more neurotypical individuals). In some embodiments, the subtype of ASD is a proton influx subtype. In some cases, the proton influx subtype may be characterized by an altered (e.g., elevated) level of proton influx in a gastrointestinal tract of the subject.

### Chorismate

### The Chorismate Hypothesis of Autism

In some aspects, the evaluation and modulation of levels of chorismate and/or one or more molecules within a chorismate metabolic pathway in a subject (e.g., within the microbiota of a subject) can be useful for therapeutic and/or diagnostic applications for ASD. Wherever chorismate is used herein, chorismic acid and/or any salt or ester of either chorismate or chorismic acid may also be used, unless the context dictates otherwise. In general, it should be understood that a reference to a compound in its ionic, protonated, salt, or ester form may encompass other forms unless the context dictates otherwise.

In some aspects, either a relative absence or abundance of chorismate and/or one or more molecules within a chorismate metabolic pathway can be detected in subjects having one or more symptoms of ASD. In some aspects, either a relative absence or abundance of bacteria likely to contain the capacity to modulate levels of chorismate and its related molecules can be detected in subjects having one or more symptoms of ASD.

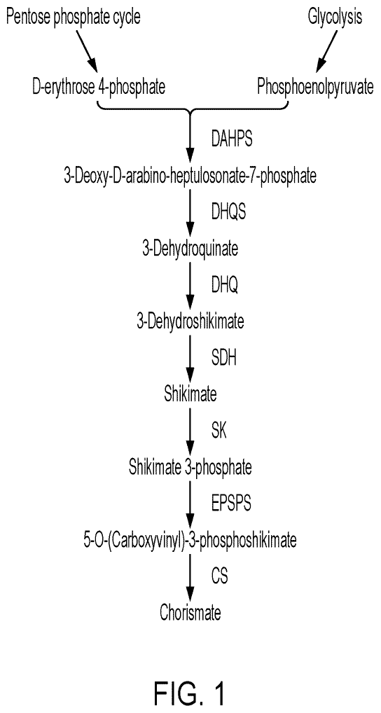

Chorismate is the end product of the shikimate pathway, which is found in microorganisms and plants, but not animals. An exemplary representation of the shikimate pathway is shown in FIG. 1. As shown in FIG. 1, the shikimate pathway comprises seven enzymatic steps: (1) 3-deoxy-D-arabino-heptulosonate-7-phosphate synthase (DAHPS) converts D-erythrose 4-phosphate (e.g., an intermediate of the pentose phosphate cycle) and phosphoenolpyruvate (e.g., an intermediate of glycolysis) to 3-deoxy-D-arabino-heptulosonate-7-phosphate (DAHP); (2) 3-dehydroquinate synthase (DHQS) converts DAHP to 3-dehydroquinate; (3) 3-dehydroquinate dehydratase (DHQ) converts 3-dehydroquinate to 3-dehydroshikimate; (4) shikimate 5-dehydrogenase (SDH) converts 3-dehydroshikimate to shikimate; (5) shikimate kinase (SK) converts shikimate to shikimate 3-phosphate; (6) 5-enolpyruvylshikimate-3-phosphate synthase (EPSPS) converts shikimate 3-phosphate to 5-O-carboxyvinyl-3-phosphoshikimate (also referred to as 5-enolpyruvylshikimate-3-phosphate (EPSP)); and (7) chorismate synthase (CS) converts 5-O-carboxyvinyl-3-phosphoshikimate to chorismate.

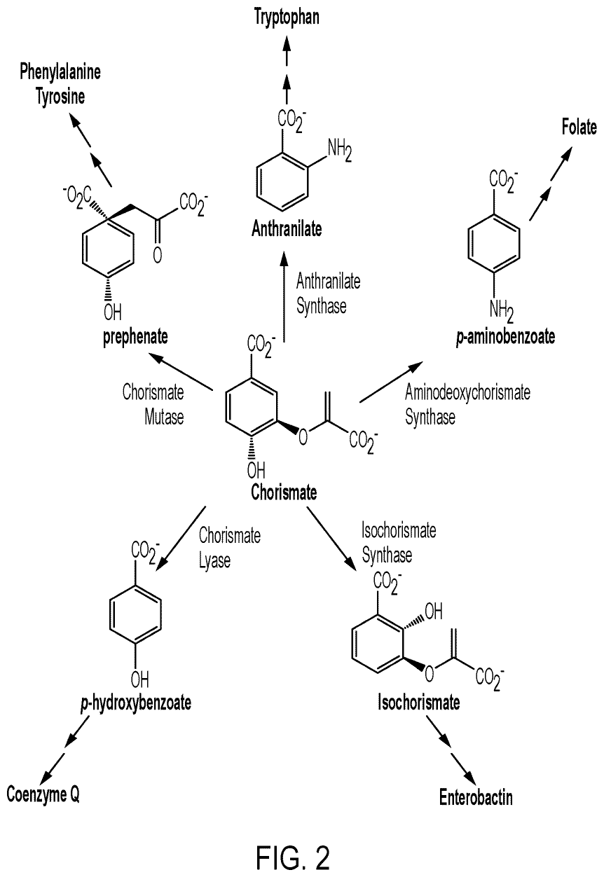

Chorismate can then be transformed into several biologically relevant aromatic molecules, including but not limited to aromatic amino acids (e.g., phenylalanine, tyrosine, tryptophan) and their derivatives, folate and its derivatives (e.g., tetrahydrofolate), siderophores (e.g., enterobactin), ubiquinols (e.g., coenzyme Q), and menaquinols. FIG. 2 shows an exemplary representation of five chorismate-dependent pathways. In one pathway, chorismate mutase converts chorismate to prephenate, which is a precursor of phenylalanine and tyrosine. In another pathway, anthranilate synthase converts chorismate to anthranilate, which is a precursor of tryptophan. In another pathway, aminodeoxychorismate synthase (also known as para-aminobenzoic acid (PABA) synthase) converts chorismate to p-aminobenzoate, which is a precursor of folate. In another pathway, isochorismate synthase converts chorismate to isochorismate, which is a precursor of enterobactin. In another pathway, chorismate lyase converts chorismate to p-hydroxybenzoate, which is a precursor of ubiquinols and/or menaquinols. According to aspects of the present invention, levels of one or more downstream metabolites of chorismate may be altered in a subject having ASD or at risk of ASD.

Because of the absence of the shikimate pathway in humans, the levels of each molecule along the shikimate pathway, as well as the pathways dependent on chorismate, are dependent on either the amount of each molecule being taken in through diet or the amount produced by gut microorganisms.

Certain chemical compounds that target one or more enzymes of the shikimate pathway may be linked to ASD. One example of this is glyphosate (also known as Roundup). Glyphosate targets the enzyme EPSPS, preventing the production of 5-O-(Carboxyvinyl)-3-phosphoshikimate (which may be converted into chorismate by chorismate synthase). One study has observed that an increase in the number of children with autism has corresponded to an increase in the amount of glyphosate applied to corn and soy. See Shaw, Elevated urinary glyphosate and clostridia metabolites with altered dopamine metabolism in triplets with autistic spectrum disorder or suspected seizure disorder: A case study, Integr Med (Encinitas), 16(1): 50-57 (2017). In some cases, glyphosate may kill beneficial bacteria, leaving bacteria (e.g., Clostridia) that can produce harmful molecules (e.g., toxins). In some cases, glyphosate may compete with Class I EPSPS enzymes but not with Class II EPSPS enzymes. Accordingly, in some cases, exposure of a subject to glyphosate may result in a decrease in species reliant on Class I EPSPS enzymes (e.g., in a gastrointestinal tract of the subject). In some embodiments, exposure of a subject to glyphosate may result in a decreased level of chorismate (e.g., in a gastrointestinal tract of the subject).

In some embodiments, exposure of a subject to glyphosate may result in the subject exhibiting one or more symptoms of ASD and/or having a higher risk of developing ASD. In certain embodiments, a method comprises determining that a subject has or is at risk of having a subtype of ASD (e.g., a chorismate subtype of ASD) based on an elevated level of glyphosate in the subject (e.g., relative to one or more neurotypical individuals).

In some cases, exposure of a subject to glyphosate may contribute to non-Hodgkin's lymphoma, mature B-cell lymphoma, mature T-cell and natural killer cell lymphoma, celiac disease, gluten intolerance, overgrowth of pathogenic bacteria, decreased beneficial microbes, impaired serotonin signaling, infertility, miscarriages, birth defects, obesity, inflammatory bowel disease, anorexia nervosa, Alzheimer's disease, Parkinson's disease, multiple sclerosis, liver disease, ADHD, amyotrophic lateral sclerosis (ALS), cancer, and/or cachexia.

Aromatic Amino Acids (and their Downstream Products)

In some embodiments, certain aromatic amino acids (e.g., tryptophan, tyrosine, phenylalanine) are non-limiting examples of molecules within a chorismate metabolic pathway.

In some embodiments, chorismate may be converted to tryptophan via a tryptophan biosynthesis pathway. In a first step of the tryptophan biosynthesis pathway, anthranilate synthase may convert chorismate to anthranilate, which is an upstream metabolite (i.e., precursor) of tryptophan. In some cases, alterations in the tryptophan biosynthesis pathway may be associated with ASD in individuals. Accordingly, in certain cases, alterations in levels of tryptophan and/or a molecule within the tryptophan biosynthesis pathway (e.g., relative to neurotypical individuals) may be associated with ASD. In some cases, a lower level of tryptophan and/or a molecule within the tryptophan biosynthesis pathway (e.g., relative to neurotypical individuals) may be associated with ASD. In some cases, a higher level of tryptophan and/or a molecule within the tryptophan biosynthesis pathway (e.g., relative to neurotypical individuals) may be associated with ASD. Accordingly, in some embodiments, a method comprises determining that a subject has or is at risk of developing at least one subtype of ASD (e.g., a chorismate subtype) based on a level of tryptophan and/or a molecule within the tryptophan biosynthesis pathway in a sample from the subject that is altered (e.g., increased, decreased) relative to the level in one or more neurotypical individuals. The sample may be any sample described herein (e.g., a fecal sample, a gastrointestinal sample, a serum sample, a CSF sample).

In some cases, alterations in levels of one or more downstream metabolites of tryptophan may be associated with ASD. Tryptophan may be further converted to downstream metabolites through at least three major pathways. In some cases, tryptophan may be converted to kynurenine or derivatives thereof through a kynurenine pathway. In some cases, tryptophan may be converted to serotonin or derivatives thereof through a serotonin pathway. In some cases, tryptophan may be converted to an indole through an indole pathway. Each of these pathways may be of potential interest to ASD.

In the serotonin pathway, the enzyme tryptophan hydroxylase (TPH) converts tryptophan into 5-hydroxytryptophan (5-HTP), which is then converted to serotonin (5-HT) by the enzyme Aromatic Amino Acid Decarboxylase (AAAD). Serotonin, a neurotransmitter, has a wide range of physiological functions in the brain and the periphery through its activation of 5-HT receptors. Serotonin itself can then be converted into several molecules, including melatonin.

In some cases, individuals with ASD may have an altered (e.g., higher, lower) level of serotonin relative to one or more neurotypical individuals. In certain cases, individuals with ASD may have a higher level of serotonin (e.g., in a serum sample) relative to one or more neurotypical individuals. In certain embodiments, the level of serotonin in a subject may be decreased by administration of probiotics and/or fructo-oligosaccharides.

In some cases, individuals with ASD may have an altered (e.g., higher, lower) level of melatonin relative to one or more neurotypical individuals. In certain cases, individuals with ASD may have a lower level of melatonin relative to one or more neurotypical individuals. In some cases, lower levels of melatonin may lead to difficulty sleeping. In certain embodiments, the level of melatonin in a subject may be increased by administration of a melatonin supplement.

In the kynurenine pathway, tryptophan is converted to kynurenine by tryptophan dioxygenase or indoleamine dioxygenase. Kynurenine may then be converted to several molecules, including but not limited to kynurenic acid, xanthurenic acid, quinolinic acid, picolinic acid, and hydroxyanthranilic acid.

In some cases, levels of kynurenine may be increased in individuals with ASD. In the gut, levels of kynurenine and other related molecules are mediated by the activity of bacterial and host IDO1 enzymes. Kynurenine as well as its downstream metabolites (including kynurenic acid, xanthurenic acid, quinolinic acid and picolinic acid) have various known biological processes, including neurotransmission, inflammation immune response, and mucosal protective effects in the gut. Other downstream metabolites of kynurenine, including hydoxyanthranilic acid, have neurotoxic effects.

In some cases, individuals with ASD may have an altered (e.g., higher, lower) level of kynurenine and/or one or more of its metabolites (e.g., downstream metabolites) relative to one or more neurotypical individuals. In certain cases, individuals with ASD may have a higher level of kynurenine and/or one or more its metabolites (e.g., downstream metabolites) relative to one or more neurotypical individuals. In certain cases, individuals with ASD may have a lower level of kynurenine and/or one or more its metabolites (e.g., downstream metabolites) relative to one or more neurotypical individuals.

Indoles are the third major category of molecules deriving from tryptophan. In the case of indoles, the microbiota play a main role in producing indoles. For example, in some cases, the bacterium Clostridium sporogenes converts tryptophan into the neurotransmitter tryptamine. In some cases, indoles may trigger signaling from AhR, which can trigger various immune responses.

In some cases, individuals with ASD may have an altered (e.g., higher, lower) level of an indole and/or one or more of its metabolites (e.g., downstream metabolites) relative to one or more neurotypical individuals. In certain cases, individuals with ASD may have a higher level of an indole and/or one or more its metabolites (e.g., downstream metabolites) relative to one or more neurotypical individuals. In certain cases, individuals with ASD may have a lower level of an indole and/or one or more its metabolites (e.g., downstream metabolites) relative to one or more neurotypical individuals.

In some embodiments, chorismate may be converted to tyrosine and/or phenylalanine. In some cases, chorismate mutase may convert chorismate to prephenate, which is a precursor of tyrosine and phenylalanine. In some cases, alterations in tyrosine biosynthesis and/or phenylalanine biosynthesis may be associated with ASD in individuals. Accordingly, in certain cases, alterations in levels of tyrosine, phenylalanine, and/or one or more of their metabolites (e.g., downstream metabolites) relative to one or more neurotypical individuals may be associated with ASD. In some cases, a lower level of tyrosine, phenylalanine, and/or one or more of their metabolites (e.g., downstream metabolites) relative to one or more neurotypical individuals may be associated with ASD. In some cases, a higher level of tyrosine, phenylalanine, and/or one or more of their metabolites (e.g., downstream metabolites) relative to one or more neurotypical individuals may be associated with ASD. Accordingly, in some embodiments, a method comprises determining that a subject has or is at risk of developing at least one subtype of ASD (e.g., a chorismate subtype) based on a level of tyrosine, phenylalanine, and/or one or more of their metabolites (e.g., downstream metabolites) in a sample from the subject that is altered (e.g., increased, decreased) relative to the level in one or more neurotypical individuals. The sample may be any sample described herein (e.g., a fecal sample, a gastrointestinal sample, a serum sample, a CSF sample).

Accordingly, in some embodiments the level of aromatic amino acids (and/or their downstream products) may be evaluated in a sample from a subject and/or modulated in the subject in order to assist in the diagnosis and/or treatment of ASD in the subject.

### Folate

In some embodiments, folate is a non-limiting example of a molecule within the chorismate metabolic pathway. In some cases, the enzyme aminodeoxychorismate synthase (also known as PABA synthase) converts chorismate toward folate biosynthesis. Folate (also known as Vitamin B9) is required for DNA and RNA synthesis as well as for the metabolism of amino acids. A lack of folate can lead to neural tube defects and, for this reason, has historically been supplemented within some transgenic foods. A portion of the human gut microbiota possess the genes capable of producing and modifying folate. There are 12 enzymes involved in converting chorismate to folate, with the activity of dihydrofolate reductase (DHFR) acting as the rate-limiting step. Folate is also dependent on Vitamin B12 (Cobalamin) for the conversion of N5-methyl tetrahydrofolate into the active form of folate, tetrahydrofolate (THF). Cobalamin also plays an important regulatory role in folate and ubiquinone biosynthesis by acting as a co-factor for an important transcriptional regulator.

In some cases, a level of folate, one or more of its derivatives (e.g., tetrahydrofolate), and/or one or more molecules involved in folate biosynthesis (e.g., cobalamin) is altered (e.g., increased, decreased) in individuals with ASD compared to neurotypical individuals. In certain cases, individuals with ASD may have increased circulating autoantibodies against folic acid receptors needed to transport folic acid across the blood-brain barrier. This may lead to a decreased uptake of folic acid in the brain and downstream consequences related to ASD pathophysiology. In certain cases, a method of treating an individual with ASD comprising administering folate, one of its derivatives, and/or one or more molecules involved in folate biosynthesis may alleviate one or more symptoms of ASD (e.g., language and communication difficulties).

Accordingly, in some embodiments the level of folate may be evaluated in a sample from a subject and/or modulated in the subject in order to assist in the diagnosis and/or treatment of ASD in the subject.

### Ubiquinols and Menaquinols

In some embodiments, ubiquinols and menaquinols are non-limiting examples of molecules within the chorismate metabolic pathway. In some cases, chorismate may branch toward ubiquinol and menaquinol production when converted to p-hydroxybenzoate by chorismate lyase. These molecules are typically found in cellular membranes and are part of electron transport chains. Ubiquinone (also referred to as Coenzyme Q) is utilized for aerobic respiration while menaquinones are commonly used by bacteria for anaerobic respiration and lactic acid fermentation. Menaquinones are also called Vitamin K2 and can vary depending on the length of their isoprenoid carbon chains. Menaquinone-4 (MK4) is the main form of Vitamin K in the brain, and serum concentrations of MK4 may be lower in children with ASD. About 75% of MK4 is produced in the gut and it is reportedly higher in males than females. Given MK4's roles in neural development, there have been some hypotheses that it may be important to ASD pathophysiology. There are also reports of a correlation between Vitamin K levels and improvement in Autism severity. In some same cases, altered levels of ubiquinol and/or menaquinol production by gut microbiota may have a disproportional effect on male brain development.

In some cases, alterations in ubiquinol and/or menaquinol biosynthesis may be associated with ASD in individuals. Accordingly, in certain cases, alterations in levels of a ubiquinol (e.g., Coenzyme Q, a ubiquinol of 6-13 carbon isoprenoid chains) and/or one or more of its metabolites (e.g., downstream metabolites) relative to one or more neurotypical individuals may be associated with ASD. In certain cases, alterations in levels of a menaquinol (e.g., menaquinone-4, a menaquinol of 6-13 carbon isoprenoid chains) and/or one or more of its metabolites (e.g., downstream metabolites) relative to one or more neurotypical individuals may be associated with ASD. In some cases, a lower level of a ubiquinol, a menaquinol, and/or a metabolite thereof (e.g., relative to neurotypical individuals) may be associated with ASD. In some cases, a higher level of a ubiquinol, a menaquinol, and/or a metabolite thereof (e.g., relative to neurotypical individuals) may be associated with ASD. Accordingly, in some embodiments, a method comprises determining that a subject has or is at risk of developing at least one subtype of ASD (e.g., a chorismate subtype) based on a level of a ubiquinol, a menaquinol, and/or a metabolite thereof in a sample from the subject that is altered (e.g., increased, decreased) relative to the level in one or more neurotypical individuals. The sample may be any sample described herein (e.g., a fecal sample, a gastrointestinal sample, a serum sample, a CSF sample).

Accordingly, in some embodiments, the level of ubiquinols and menaquinols may be evaluated in a sample from a subject and/or modulated in the subject in order to assist in the diagnosis and/or treatment of ASD in the subject.

### Siderophores

In some embodiments, siderophores are non-limiting examples of molecules within the chorismate metabolic pathway. A non-limiting example of a siderophore is enterobactin. In some cases, an isochorismate synthase enzyme (e.g., EntC) converts chorismate to isochorismate in the first step towards producing enterobactin from chorismate. Siderophores are typically thought of as virulence factors in a fight between humans and bacteria for metals. However, some studies have shown a beneficial role for enterobactin on the growth of C. elegans. This phenotype in C. elegans was linked to mitochondrial function, and mitochondrial dysfunction has been observed in ASD children.

In some cases, levels of siderophores (e.g., enterobactin) may be altered (e.g., increased, decreased) in individuals with ASD relative to neurotypical individuals. In some cases, a lower level of a siderophore (e.g., enterobactin) relative to neurotypical individuals may be associated with ASD. In some cases, a higher level of a siderophore (e.g., enterobactin) relative to neurotypical individuals may be associated with ASD. Accordingly, in some embodiments, a method comprises determining that a subject has or is at risk of developing at least one subtype of ASD (e.g., a chorismate subtype) based on a level of a siderophore (e.g., enterobactin) in a sample from the subject that is altered (e.g., increased, decreased) relative to the level in one or more neurotypical individuals. The sample may be any sample described herein (e.g., a fecal sample, a gastrointestinal sample, a serum sample).

Accordingly, in some embodiments the level of siderophores may be evaluated in a sample from a subject and/or modulated in the subject in order to assist in the diagnosis and/or treatment of ASD in the subject.

### Chorismate Subtype & Interventions

Some embodiments are directed to methods and compositions for diagnosing and/or treating a chorismate subtype of ASD.

According to some embodiments, a method of assisting in the diagnosis of a subject having one or more symptoms of ASD and/or of comorbidities of ASD comprises determining a level of chorismate and/or a molecule within a chorismate metabolic pathway in a sample obtained from the subject. In some embodiments, the method further comprises determining, if the level of chorismate and/or the molecule within the chorismate metabolic pathway is higher or lower than a threshold value, that the subject has or is at risk of developing ASD (e.g., a chorismate subtype of ASD).

In some embodiments, the molecule within the chorismate metabolic pathway is an upstream or downstream metabolite of chorismate. Non-limiting examples of upstream metabolites of chorismate include D-erythrose 4-phosphate, phosphoenolpyruvate, 3-deoxy-D-arabino-heptulosonate-7-phosphate, 3-dehydroquinate, 3-dehydroshikimate, shikimate, shikimate 3-phosphate, and 5-O-(carboxyvinyl)-3-phosphoshikimate. Non-limiting examples of downstream metabolites of chorismate include aromatic amino acids (e.g., tryptophan, tyrosine, phenylalanine) and metabolites thereof (e.g., serotonin, melatonin, kyneurenine, an indole), folate and derivatives thereof (e.g., tetrahydrofolate), ubiquinols (e.g., coenzyme Q), menaquinols, and siderophores (e.g., enterobactin).

In some embodiments, determining the level of chorismate and/or the molecule within the chorismate metabolic pathway comprises determining an abundance of one or more genes within the superpathway of chorismate metabolism present in a sample obtained from the subject. In certain embodiments, determining the abundance of the one or more genes comprises amplifying at least a portion of one or more nucleic acids present in the sample to form a plurality of amplicons. The one or more nucleic acids may be amplified according to any nucleic acid amplification method known in the art (e.g., PCR, qPCR) to form a plurality of amplicons. In some embodiments, determining the abundance of the one or more genes further comprises sequencing one or more amplicons of the plurality of amplicons. The one or more amplicons may be sequenced according to any nucleic acid sequencing method known in the art (e.g., a long-read sequencing method, a short-read sequencing method).

In some instances, determining the abundance of one or more genes within the superpathway of chorismate metabolism present in a sample obtained from the subject comprises sequencing at least a portion of 16S ribosomal RNA (rRNA) of microbes present in the sample (e.g., performing 16S rRNA gene amplicon (16S) sequencing). In some instances, determining the abundance of one or more genes within the superpathway of chorismate metabolism present in a sample obtained from the subject comprises performing shotgun metagenomic sequencing to sequence the metagenomic content of the sample.

In some embodiments, determining the level of chorismate and/or the molecule within the chorismate metabolic pathway comprises measuring the level of chorismate and/or the molecule within the chorismate metabolic pathway (e.g., using HPLC, LC-MS, GC-MS, and/or MALDI-TOF).

Some embodiments are directed to methods of treating a subject having one or more symptoms of ASD and/or of comorbidities of ASD or at risk of developing ASD. Examples of comorbidities of ASD include, but are not limited to, obsessive-compulsive disorder (OCD), attention deficit disorder (ADD), attention deficit hyperactivity disorder (ADHD), epilepsy, schizophrenia, sleep disorders, gastrointestinal disorders, obesity, irritability, anxiety, depression, and eating disorders.

In certain embodiments, the subject has the chorismate subtype of ASD. In some cases, a sample obtained from the subject may comprise a compositional abundance of one or more molecules and/or microbial features indicating that the subject may be likely to respond to a chorismate-based intervention. In certain cases, the one or more molecules and/or microbial features comprise enterobacteriales, L-arginine degradation products, and/or enterobactin. In certain cases, the one or more molecules and/or microbial features comprise one or more metabolites of the superpathway of chorismate metabolism, including but not limited to shikimate, chorismate, tryptophan, serotonin, melatonin, phenylalanine, tyrosine, folate (e.g., tetrahydrofolate), a ubiquinol (e.g., Coenzyme Q), and a menaquinol. In some cases, a sample obtained from the subject may comprise an abundance of one or more genes (e.g., one or more genes within the superpathway of chorismate metabolism) indicating that the subject may be likely to respond to a chorismate-based intervention.

In some embodiments, the method of treating the subject comprises modulating a level of chorismate and/or a molecule within a chorismate metabolic pathway in the subject to alleviate one or more symptoms of ASD and/or of comorbidities of ASD or to reduce the risk of developing ASD. In some embodiments, modulating the level comprises increasing the level. In some embodiments, modulating the level comprises decreasing the level. In some cases, the level is modulated in the gut of the subject.

In some embodiments, modulating the level of chorismate and/or the molecule within the chorismate metabolic pathway comprises administering to the subject a composition comprising chorismate, chorismic acid, and/or a salt or ester of either thereof. In some embodiments, modulating the level of chorismate and/or the molecule within the chorismate metabolic pathway comprises administering to the subject a composition comprising a chorismate prodrug. In some embodiments, modulating the level of chorismate and/or the molecule within the chorismate metabolic pathway comprises administering to the subject a composition comprising a chorismate metabolite (and/or another molecule involved in a chorismate metabolic pathway). In certain embodiments, the composition comprises chorismate, chorismic acid, and/or a salt or ester of either thereof, and one or more chorismate prodrugs. In certain embodiments, the composition comprises chorismate, chorismic acid, and/or a salt or ester of either thereof, and one or more chorismate metabolites (and/or one or more other molecules involved in a chorismate metabolic pathway). In certain embodiments, the composition comprises one or more chorismate prodrugs and one or more chorismate metabolites (and/or one or more other molecules involved in a chorismate metabolic pathway). The one or more chorismate metabolites may comprise one or more upstream chorismate metabolites (e.g., shikimate) and/or one or more downstream chorismate metabolites (e.g., tryptophan, phenylalanine, tyrosine, serotonin, melatonin, an indole, a ubiquinol, a menaquinol such as menaquinone-4, tetrahydrofolate, enterobactin). The one or more other molecules involved in a chorismate metabolic pathway may comprise one or more enzymes (e.g., one or more chorismate branchpoint enzymes) and/or one or more molecules involved in regulation of a chorismate metabolic pathway (e.g., adenosylcobalamin, cobalamin (also known as Vitamin B12)).

In some embodiments, the composition may be provided as a pharmaceutical composition comprising one or more pharmaceutically acceptable buffers, salts, additives, or other agents. In some embodiments, the composition (e.g., pharmaceutical composition) may be administered enterally, parenterally, or via any other suitable route. In some embodiments, the composition (e.g., pharmaceutical composition) may be administered orally, rectally, intravenously, intramuscularly, via inhalation, and/or topically. In some instances, the composition (e.g., pharmaceutical composition) may be in tablet form, in gelcap form, in capsule form, in liquid form, or in any other form suitable for oral administration. In some cases, the composition (e.g., pharmaceutical composition) may comprise a coating or other system configured to provide for pH-dependent and/or time-dependent release of an active component of the composition. In certain instances, the composition (e.g., pharmaceutical composition) may comprise a capsule comprising a pH-dependent colonic release coating. In some instances, the composition (e.g., pharmaceutical composition) is configured for intravenous administration.

In some embodiments, a composition (e.g., a pharmaceutical composition) comprises a pharmaceutically acceptable carrier (e.g., a diluent, adjuvant, excipient, or vehicle with which the composition is administered). Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers.

In some embodiments, a composition (e.g., a pharmaceutical composition) is administered to a subject in an effective amount, for example in an amount sufficient to alleviate (e.g., delay or reduce the onset, progression, and/or severity of) one or more symptoms of ASD and/or of comorbidities of ASD. In some embodiments, the composition (e.g., pharmaceutical composition) is administered to a subject in an amount sufficient to reduce a risk of a subject developing ASD. In some instances, modulating the level of chorismate and/or the molecule within the chorismate metabolic pathway comprises modifying the subject's diet to increase chorismate intake.

In certain embodiments, modulating the level of chorismate and/or the molecule within the chorismate metabolic pathway comprises administering to the subject a composition (e.g., a pharmaceutical composition) that modulates (e.g., increases, decreases) expression and/or activity of one or more enzymes in the chorismate metabolic pathway. In some embodiments, the composition (e.g., pharmaceutical composition) comprises an agonist, antagonist, activator, and/or inhibitor of the one or more enzymes. In some embodiments, the one or more enzymes comprise a chorismate branchpoint enzyme. In some embodiments, chorismate mutase is modulated. In some embodiments, chorismate lyase is modulated (e.g., inhibited). In some embodiments, isochorismate synthase is modulated (e.g., inhibited). In some embodiments, aminodeoxychorismate synthase (also known as PABA synthase) is modulated (e.g., inhibited). In some embodiments, anthranilate synthase is modulated.

In certain embodiments, modulating the level of chorismate and/or the molecule within the chorismate metabolic pathway comprises administering to the subject a composition (e.g., a pharmaceutical composition) that modulates (e.g., increases, decreases) an amount of a microbe that produces chorismate and/or the molecule within the chorismate metabolic pathway in a gastrointestinal tract of the subject. In some embodiments, the composition (e.g., pharmaceutical composition) comprises one or more antibiotics, prebiotic compounds, and/or probiotic compounds. In certain cases, the one or more antibiotics comprise vancomycin, fidaxomicin, and/or metronidazole.

In some embodiments, a method of reducing a risk of developing ASD and/or treating one or more symptoms of ASD and/or of comorbidities of ASD comprises providing microbes that mimic a typical representation of chorismate-related genes and/or that modulate the microbiome in a manner that restores a typical representation of chorismate-related genes. In some embodiments, providing a supplement of one or more chorismate-related molecules can be useful to direct the microbiome toward a typical presentation of chorismate-related genes and therefore help treat one or more of the symptoms of autism.

The composition (e.g., pharmaceutical composition) may be administered to a subject of any age (e.g., a child, an adult). In some embodiments, the composition (e.g., pharmaceutical composition) may be administered chronically. In certain embodiments, the composition (e.g., pharmaceutical composition) may be administered over a period of more than 30 days, 3 months, 6 months, 9 months, 1 year, 3 years, 5 years, 10 years, or 20 years. In certain embodiments, the composition (e.g., pharmaceutical composition) may be administered over a period of 1-3 months 1-6 months, 1-9 months 1 month to 1 year, 1 month to 3 years, 1 month to 5 years, 1 month to 10 years, 1 month to 20 years, 3-6 months, 3-9 months, 3 months to 1 year, 3 months to 3 years, 3 months to 5 years, 3 months to 10 years, 3 months to 20 years, 6-9 months, 6 months to 1 year, 6 months to 3 years, 6 months to 5 years, 6 months to 10 years, 6 months to 20 years, 1-3 years, 1-5 years, 1-10 years, 1-20 years, 3-5 years, 3-10 years, 3-20 years, 5-10 years, 5-20 years, or 10-20 years. In some embodiments, a composition (e.g., a pharmaceutical composition) is administered daily, weekly, monthly, or at shorter or longer time intervals. In some embodiments, a composition (e.g., a pharmaceutical composition) is administered until one or more biomarkers indicative of ASD risk are reduced. In some embodiments, a composition (e.g., a pharmaceutical composition) is administered in an amount sufficient to alleviate one or more symptoms of ASD and/or of comorbidities of ASD or to reduce the risk of developing ASD.

### Microbial Organisms

According to some embodiments, a method of assisting in the diagnosis of a subject having one or more symptoms of ASD and/or of comorbidities of ASD comprises determining an abundance of one or more microbial features (e.g., microbial organisms) and/or their byproducts (e.g., toxins) in a sample obtained from the subject. In some embodiments, the method further comprises determining, if the abundance of the one or more microbial features (e.g., microbial organisms) and/or their byproducts (e.g., toxins) is higher or lower than a threshold value, that the subject has or is at risk of developing ASD (e.g., a Sarcina subtype of ASD).

Non-limiting examples of microbial organisms include Sarcina, Prevotella (e.g., P. stercorea), Bifidobacteria, Actinobacteria, Bacteroidetes, Proteobacteria, Eggerthella lenta, Enterobacteriaceae, Aeromonas, Agrobacterium, Enterococcus, Actinobacillus, Lactobacillus, and/or Streptococcus. In certain embodiments, the microbial organism is a Sarcina bacterium. In certain embodiments, the microbial organism comprises Sarcina ventriculi (also known as Clostridium ventriculi). In certain embodiments, the microbial organism comprises Clostridium perfringens. In some cases, a subject whose sample has an elevated level of Sarcina bacteria (e.g., relative to neurotypical individuals) may be referred to as having the Sarcina subtype of ASD.

Some embodiments are directed to methods of treating a subject having one or more symptoms of ASD and/or of comorbidities of ASD or at risk of developing ASD. In certain embodiments, the subject has the Sarcina subtype of ASD. In some embodiments, the method comprises modulating a level of one or more microbial organisms (e.g., S. ventriculi and/or other Sarcina bacteria) in the subject to alleviate one or more symptoms of ASD and/or of comorbidities of ASD or to reduce the risk of developing ASD. In some cases, the level of the one or more microbial organisms is modulated in the gut of the subject. In some embodiments, modulating the level of the one or more microbial organisms comprises decreasing the level. In certain instances, modulating the level comprises administering a composition comprising one or more antibiotics and/or phage therapy to the subject. In some embodiments, modulating the level comprises increasing the level. In certain instances, modulating the level of the one or more microbial organisms comprises administering to the subject a composition comprising one or more prebiotics and/or probiotics.

In some embodiments, the composition (e.g., a composition comprising one or more antibiotics, bacteriophages, prebiotic compounds, and/or probiotic compounds) may be provided as a pharmaceutical composition comprising one or more pharmaceutically acceptable buffers, salts, additives, or other agents. In some embodiments, the composition (e.g., pharmaceutical composition) may be administered enterally, parenterally, or via any other suitable route. In some embodiments, the composition (e.g., pharmaceutical composition) may be administered orally, rectally, intravenously, intramuscularly, via inhalation, and/or topically. In some instances, the composition (e.g., pharmaceutical composition) may be in tablet form, in gelcap form, in capsule form, in liquid form, or in any other form suitable for oral administration. In some cases, the composition (e.g., pharmaceutical composition) may comprise a coating or other system configured to provide for pH-dependent and/or time-dependent release of an active component of the composition. In certain instances, the composition (e.g., pharmaceutical composition) may comprise a capsule comprising a pH-dependent colonic release coating. In some instances, the composition (e.g., pharmaceutical composition) is configured for intravenous administration.

In some embodiments, a composition (e.g., a pharmaceutical composition) comprises a pharmaceutically acceptable carrier (e.g., a diluent, adjuvant, excipient, or vehicle with which the composition is administered). Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers.

In some embodiments, a composition (e.g., a pharmaceutical composition) is administered to a subject in an effective amount, for example in an amount sufficient to alleviate (e.g., delay or reduce the onset, progression, and/or severity of) one or more symptoms of ASD and/or of comorbidities of ASD. In some embodiments, the composition (e.g., pharmaceutical composition) is administered to a subject in an amount sufficient to reduce a risk of a subject developing ASD.

The composition (e.g., pharmaceutical composition) may be administered to a subject of any age (e.g., a child, an adult). In some embodiments, the composition (e.g., pharmaceutical composition) may be administered chronically. In certain embodiments, the composition (e.g., pharmaceutical composition) may be administered over a period of more than 30 days, 3 months, 6 months, 9 months, 1 year, 3 years, 5 years, 10 years, or 20 years. In certain embodiments, the composition (e.g., pharmaceutical composition) may be administered over a period of 1-3 months 1-6 months, 1-9 months 1 month to 1 year, 1 month to 3 years, 1 month to 5 years, 1 month to 10 years, 1 month to 20 years, 3-6 months, 3-9 months, 3 months to 1 year, 3 months to 3 years, 3 months to 5 years, 3 months to 10 years, 3 months to 20 years, 6-9 months, 6 months to 1 year, 6 months to 3 years, 6 months to 5 years, 6 months to 10 years, 6 months to 20 years, 1-3 years, 1-5 years, 1-10 years, 1-20 years, 3-5 years, 3-10 years, 3-20 years, 5-10 years, 5-20 years, or 10-20 years. In some embodiments, a composition (e.g., a pharmaceutical composition) is administered daily, weekly, monthly, or at shorter or longer time intervals. In some embodiments, a composition (e.g., a pharmaceutical composition) is administered until one or more biomarkers (e.g., levels of a Sarcina bacterium) indicative of ASD risk are reduced. In some embodiments, a composition (e.g., a pharmaceutical composition) is administered in an amount sufficient to alleviate one or more symptoms of ASD and/or of comorbidities of ASD or to reduce the risk of developing ASD.

### Proton Flux

According to some embodiments, a method of assisting in the diagnosis of a subject having one or more symptoms of ASD and/or of comorbidities of ASD comprises determining a level of proton flux in a gastrointestinal tract of the subject from a sample obtained from the subject. In some embodiments, the method further comprises determining, if the level of proton flux is higher than a threshold value, that the subject has or is at risk of developing ASD (e.g., a proton flux subtype of ASD).

Some embodiments are directed to methods of treating a subject having one or more symptoms of ASD and/or of comorbidities of ASD or at risk of developing ASD. In certain embodiments, the subject has the proton flux subtype of ASD. In some embodiments, the method comprises modulating a gastrointestinal pH of the subject to alleviate one or more symptoms of ASD and/or of comorbidities of ASD or to reduce the risk of developing ASD. In some embodiments, modulating the pH comprises increasing the pH. In some embodiments, modulating the pH comprises decreasing the pH. In some embodiments, modulating the gastrointestinal pH comprises administering to a subject a composition or treatment configured to modulate the gastrointestinal pH of the subject. In some embodiments, the composition or treatment comprises one or more proton-pump inhibitors. Non-limiting examples of proton-pump inhibitors include omeprazole, lansoprazole, pantoprazole, rabeprazole, esomeprazole, dexlansoprazole, and derivatives thereof. In some embodiments, the composition or treatment comprises bismuth subsalicylate or derivatives thereof formulated for colonic release.

In some embodiments, the composition (e.g., a composition comprising one or more proton-pump inhibitors and/or bismuth subsalicylate or derivatives thereof) may be provided as a pharmaceutical composition comprising one or more pharmaceutically acceptable buffers, salts, additives, or other agents. In some embodiments, the composition (e.g., pharmaceutical composition) may be administered enterally, parenterally, or via any other suitable route. In some embodiments, the composition (e.g., pharmaceutical composition) may be administered orally, rectally, intravenously, intramuscularly, via inhalation, and/or topically. In some instances, the composition (e.g., pharmaceutical composition) may be in tablet form, in gelcap form, in capsule form, in liquid form, or in any other form suitable for oral administration. In some cases, the composition (e.g., pharmaceutical composition) may comprise a coating or other system configured to provide for pH-dependent and/or time-dependent release of an active component of the composition. In certain instances, the composition (e.g., pharmaceutical composition) may comprise a capsule comprising a pH-dependent colonic release coating. In some instances, the composition (e.g., pharmaceutical composition) is configured for intravenous administration.

In some embodiments, a composition (e.g., a pharmaceutical composition) comprises a pharmaceutically acceptable carrier (e.g., a diluent, adjuvant, excipient, or vehicle with which the composition is administered). Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers.

In some embodiments, a composition (e.g., a pharmaceutical composition) is administered to a subject in an effective amount, for example in an amount sufficient to alleviate (e.g., delay or reduce the onset, progression, and/or severity of) one or more symptoms of ASD and/or of comorbidities of ASD. In some embodiments, the composition (e.g., pharmaceutical composition) is administered to a subject in an amount sufficient to reduce a risk of a subject developing ASD.

The composition (e.g., pharmaceutical composition) may be administered to a subject of any age (e.g., a child, an adult). In some embodiments, the composition (e.g., pharmaceutical composition) may be administered chronically. In certain embodiments, the composition (e.g., pharmaceutical composition) may be administered over a period of more than 30 days, 3 months, 6 months, 9 months, 1 year, 3 years, 5 years, 10 years, or 20 years. In certain embodiments, the composition (e.g., pharmaceutical composition) may be administered over a period of 1-3 months 1-6 months, 1-9 months 1 month to 1 year, 1 month to 3 years, 1 month to 5 years, 1 month to 10 years, 1 month to 20 years, 3-6 months, 3-9 months, 3 months to 1 year, 3 months to 3 years, 3 months to 5 years, 3 months to 10 years, 3 months to 20 years, 6-9 months, 6 months to 1 year, 6 months to 3 years, 6 months to 5 years, 6 months to 10 years, 6 months to 20 years, 1-3 years, 1-5 years, 1-10 years, 1-20 years, 3-5 years, 3-10 years, 3-20 years, 5-10 years, 5-20 years, or 10-20 years. In some embodiments, a composition (e.g., a pharmaceutical composition) is administered daily, weekly, monthly, or at shorter or longer time intervals. In some embodiments, a composition (e.g., a pharmaceutical composition) is administered until one or more biomarkers indicative of ASD risk (e.g., high proton flux) are reduced. In some embodiments, a composition (e.g., a pharmaceutical composition) is administered in an amount sufficient to alleviate one or more symptoms of ASD and/or of comorbidities of ASD or to reduce the risk of developing ASD.

### Techniques and Systems for Evaluating a Subject's Risk of Having or Developing ASD

In some embodiments, once one or more characteristics (e.g., abundance of molecules and/or microbial features) are determined from a sample from a subject, the one or more characteristics may be analyzed with a risk analysis facility to evaluate the subject's risk of having or developing ASD and/or for identifying a treatment to provide to the subject. The risk analysis facility may be implemented in software, such as in executable instructions that are executed by one or more servers, one or more laboratory devices (e.g., a laboratory device that receives, prepares, and analyzes a sample, such as a fecal sample), one or more personal computing devices or mobile computing devices, or other devices.

In some such embodiments that operate with a risk analysis facility, the analysis of a subject's risk for having or developing ASD and/or the analysis for identifying a treatment may be performed in some embodiments using one or more rules. For example, when a rule or combination of rules are met, a subject may be determined have a higher or lower risk of ASD or may be found to be a candidate for a particular treatment or not. Such rules may, in some cases, relate to the level of one or more molecules and/or type(s) of microbes detected within the subject's microbiome or amounts or relative amounts of the molecules and/or microbes. As one specific example, if a subject's fecal sample demonstrates a relative abundance of a particular microbe within the fecal sample, the patient may be found to be at increased risk for ASD.

In some embodiments, in addition to or as an alternative to a rules-based analysis, a risk analysis facility may leverage a trained machine learning model to evaluate a subject's risk of having or developing ASD or to identify a treatment to provide to the patient. In some embodiments that implement a machine learning model, such a machine learning model may be trained based on one or more characteristics (e.g., abundance of molecules and/or microbial features) of a population of people that were diagnosed with ASD and a population of people that were not diagnosed with ASD and, as a result of the training, may have learned relationships between sample characteristics (e.g., abundance of molecules and/or microbial features) and presence or absence of ASD and/or the success or lack of success of certain characteristics. In some such embodiments, once one or more characteristics are obtained through analyzing a sample, the one or more characteristics may be input to the machine learning model. In some such embodiments, the machine learning model may respond by outputting a classification of the subject's microbiome or of the subject into a particular class, of a set of classes. Each class may be associated with a risk of or a particular risk level of having or developing ASD and/or being a candidate or not for a particular treatment. In other embodiments, each class may additionally or alternatively be related to a subtype of ASD, where each subtype of ASD corresponds to a risk level, a type of ASD a subject may have or develop, a likely combination of symptoms a subject may experience, a severity of symptoms, or other characteristics of an ASD condition the subject may be at risk of having or developing. In some embodiments, rather than outputting a single class, a machine learning engine may output a set of one or more classes and, for each class, a confidence level associated with the class indicating likelihood computed by the machine learning engine that the subject is correctly classified into that class. The likelihoods may then be evaluated, each by a risk analysis facility, to identify one or more of the classes into which the subject will be identified as being classified. For example, the class with the highest likelihood may be identified as the class into which a subject fits. As another example, only the classes with likelihoods above a first threshold may be analyzed further, and a determination may be made from among those classes which is/are the class(es) the subject will be identified as matching. For example, the highest may be identified, or the one or more classes that are more than a threshold amount more likely than other classes.

In embodiments that use a risk analysis facility, once a subject is identified by the risk analysis facility as fitting some criterion or criteria (e.g., through a rules analysis, a machine learning analysis such as a classification, or another analysis), then the risk analysis facility may initiate a diagnostic or treatment workflow consistent with the criterion/criteria. For example, a workflow may include outputting to a clinician (e.g., lab technician, physician, nurse, etc.) a determination of the criteria/criterion met and associate risks, diagnoses, treatment recommendations, or other information associated with the satisfaction of those criteria/criterion. As another example, the workflow may include prescribing a treatment, such as initiating an order for a prescription for a treatment associated with the criteria/criterion or with the risk or treatment recommendation. As another example, the workflow may include ordering a follow-up analysis, such as a developmental monitoring or screening or other evaluation by a physician (e.g., psychologist, neurologist, or other physician) to confirm the risk or treatment recommendation or obtain additional information regarding the risk to inform a treatment plan. In embodiments that identify a treatment to provide to a subject to reduce a risk of developing or to mitigate or alleviate the symptoms of ASD or of comorbidities of ASD, various suitable therapies may be leveraged. In some embodiments, the therapies may be related to microbial features, such as microbial features used in evaluating a subject's risk or having or developing ASD. Such therapies may include a treatment to introduce one or more particular microbes into a patient's microbiome or one or more compounds (e.g., drugs, medications, etc.) to encourage growth of particular microbes. Such a treatment to encourage growth may include administration of a probiotic or prebiotic. Such therapies may additionally or alternatively include a treatment to attempt to remove microbes from a subject's microbiome or discourage growth of particular microbes, such as through antibiotics or phage therapy. Such therapies may further additionally or alternatively include a treatment to introduce or increase a prevalence or, or remove or decrease a prevalence of, microbial molecules or other compounds associated with microbes (e.g., beneficial compounds that are produced by or stem from a microbe, or toxins or harmful or non-beneficial compounds that are produced by or stem from a microbe). Therapies based on microbial features may include therapies to encourage the patient's microbiome to become more similar to a target microbiome, e.g., through having a composition of microbes that is more similar to the target microbiome's composition than a patient's current microbiome composition.

### Example 1

In this Example, a quantitative meta-analysis of existing ASD microbiome data was conducted. The effects that 16S ribosomal RNA gene amplicon (16S) sequencing methods, sex, geographic location, control type, and age have on detecting differences in the ASD microbiome, as well as how these factors may influence the results of machine learning prediction of ASD status, were evaluated. The data was derived from thirteen published case-control cohorts and one curated cohort from the American Gut Project (AGP). The cohorts were analyzed individually and as a whole to draw conclusions about how the ASD gut microbiome differs from developmentally neurotypical individuals.

### Included Studies and Sample Demographics

Following the Preferred Reporting Items for Systematic reviews and Meta-Analyses (PRISMA), a dataset consisting of fourteen different case control cohorts was curated. The resulting dataset had a total of 1,740 samples, of which 888 were diagnosed with ASD and 852 were typically developing controls. The largest cohort was manually curated from participants from the AGP (n=524), with other large cohorts from Dan et al. (n=286) and Chen 2020 (n=246). Control subjects were selected from the AGP in a manner strictly controlling for technical variability while finding best matches based on a variety of selected variables. This approach resulted in a control cohort that largely reflected the demographics of the AGP's ASD subjects. In total, a majority of samples were from males, which follows the general trend of increased rates of ASD amongst men. The studies typically included participants between the ages of 2-6 years old, with notable exceptions being Kong et al. and Kang et al., which included older individuals. Thirteen of the cohorts represented samples from the United States (n=1079) and China (n=866), and one study, Zurita et al., 2019, was conducted in Ecuador. In addition, a large number of the samples (n=1280) were processed targeting the 16S V4 hypervariable region, while 357 samples were sequenced from the 16S V3-V4 region and 103 were processed from the V1-V2 region. Most studies utilized an age and sex matched sample from unrelated controls, while two studies utilized siblings and two studies utilized the mothers of ASD children.

### Evaluating Differences in ASD Microbiome Diversity

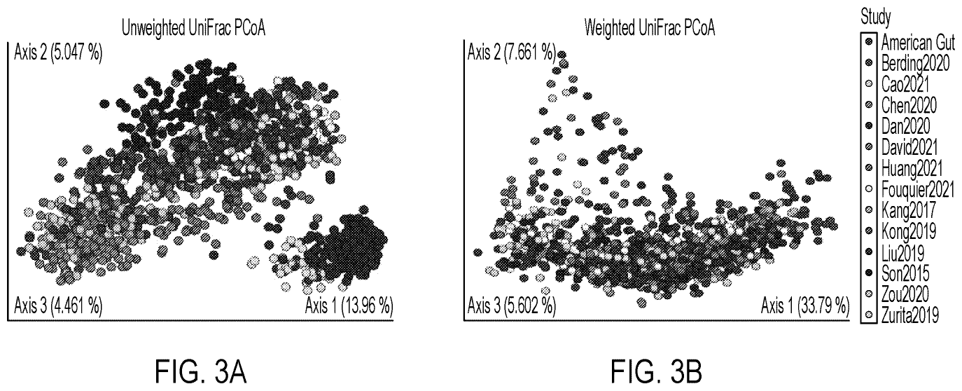

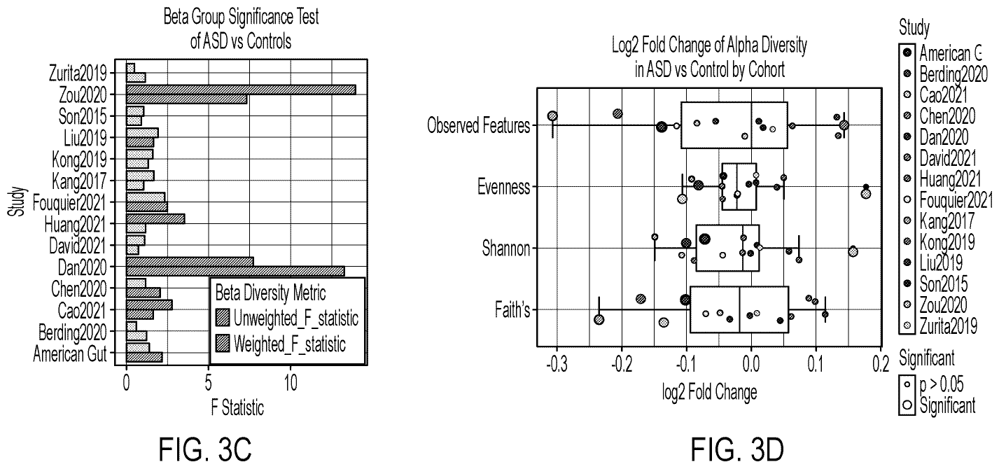

To address the question of whether microbiome diversity is altered in ASD, multiple alpha and beta-diversity metrics were assessed on both a per-study and an aggregated dataset approach. For beta-diversity, two commonly utilized distance metrics—unweighted and weighted UniFrac—were tested. These metrics are phylogenetically-informed metrics that were not used in all of the original reports of the datasets. In the aggregate, a significant clustering by study was observed for both the unweighted and weighted UniFrac distance metrics, though the effect was notably stronger for the unweighted UniFrac distance (unweighted UniFrac PERMANOVA P<0.001, pseudo-F=42.15; weighted UniFrac PERMANOVA P<0.001, pseudo-F=18.54) (FIGS. 3A-3B). This observation may indicate that study-unique microbes of low abundance can increase study separation and drive study discrepancies. While less striking than the study effect, significant clustering by ASD status was observed in unweighted UniFrac distance (PERMANOVA P<0.001, pseudo-F=3.99), but not weighted UniFrac distance (PERMANOVA P=0.162, pseudo-F statistic=1.48). Strong separation was also observed related to the 16S hypervariable region used, highlighting the importance of accounting for which variable region was used. Differences related to sequencing depth and geographic location in unweighted UniFrac distances were also observed, though these results were confounded by the effect of variable region and study.

Given that there was a strong confounding effect on the beta-diversity results driven by which study the samples were coming from, each cohort was further analyzed individually. Eight of the fourteen cohorts showed statistically significant differences (PERMANOVA P<0.05) in the ASD gut microbiome compared to neurotypical controls from either the weighted or unweighted UniFrac distance metrics (FIG. 3C). These results indicated that there is variability in detecting beta-diversity differences between ASD and controls, though it can be detected in a majority of studies and when analyzing studies in aggregate.

Each study was also analyzed for the effect of ASD status on within-sample microbial diversity. Four alpha-diversity metrics were utilized: Faith's phylogenetic diversity, Shannon diversity, evenness, and observed features. For all metrics, the study effect was large, requiring analysis of each of the fourteen cohorts individually. For each of the four alpha-diversity metrics utilized, there were at least three studies that showed statistically significant differences in the alpha-diversity values between ASD and neurotypical controls (Students' t-test, P<0.05) (FIG. 3D). Of the 14 cohorts, 7 showed statistically significant differences in alpha-diversity between ASD and neurotypical controls in at least one of the four metrics (FIG. 3D). In general, studies showed modestly decreased alpha-diversity among ASD subjects (median ASD/control difference in observed features=+0.04%, evenness=−2.5%, Shannon=−0.09%, and Faith's=−1.2%). These results suggested an unclear association between ASD and alpha-diversity. Potential reasons for this discrepancy are later described in this Example.

### Differences in Microbial Relative Abundance Between ASD and Controls

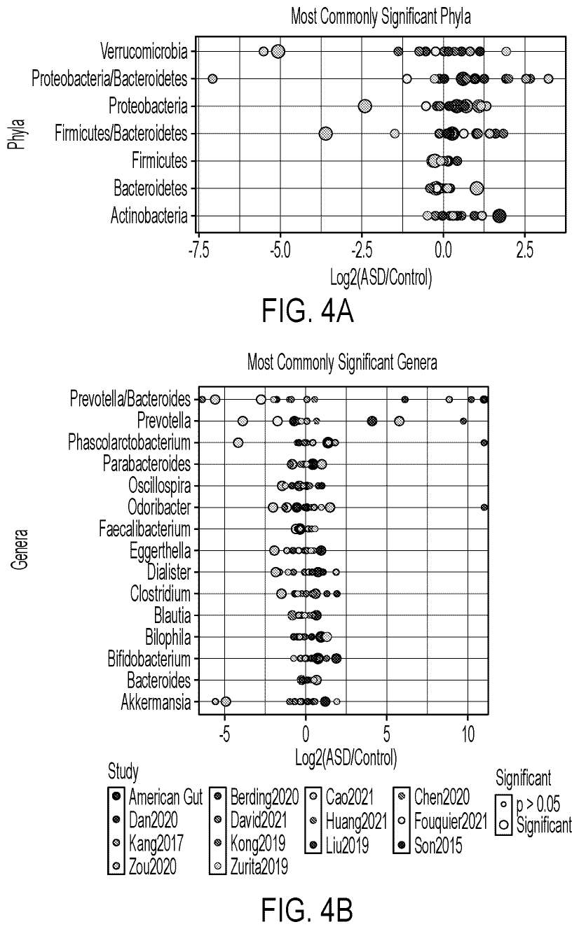

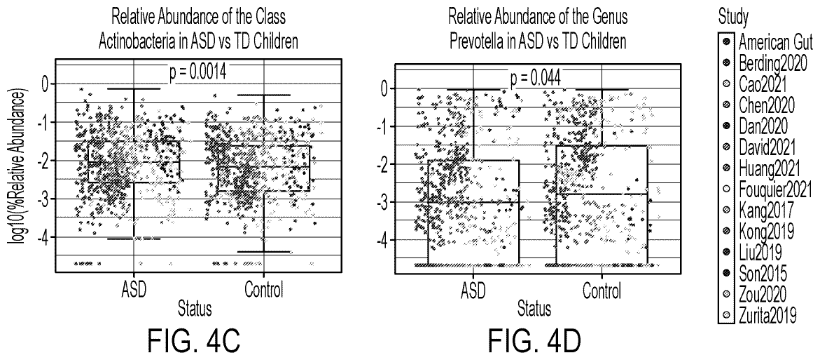

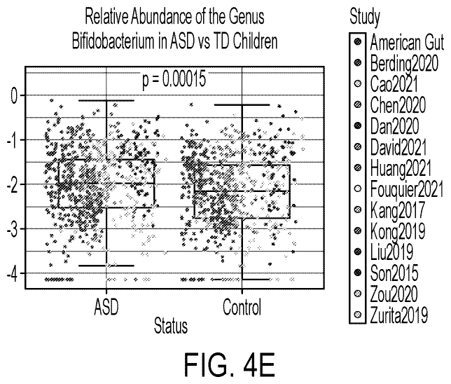

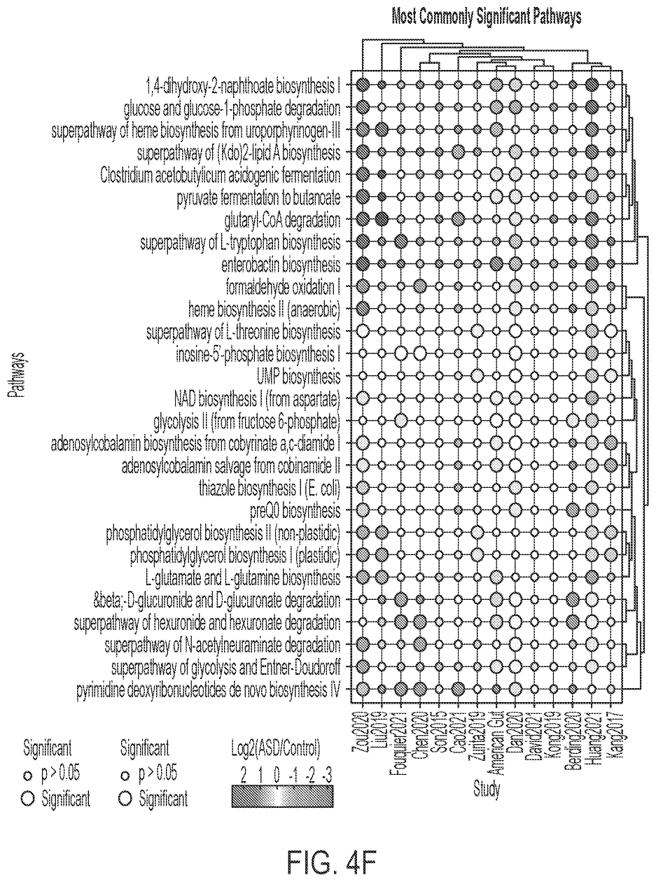

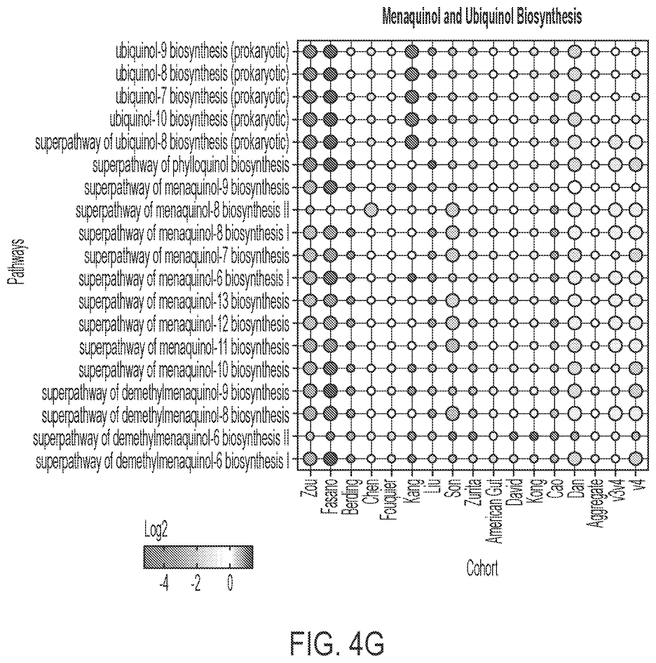

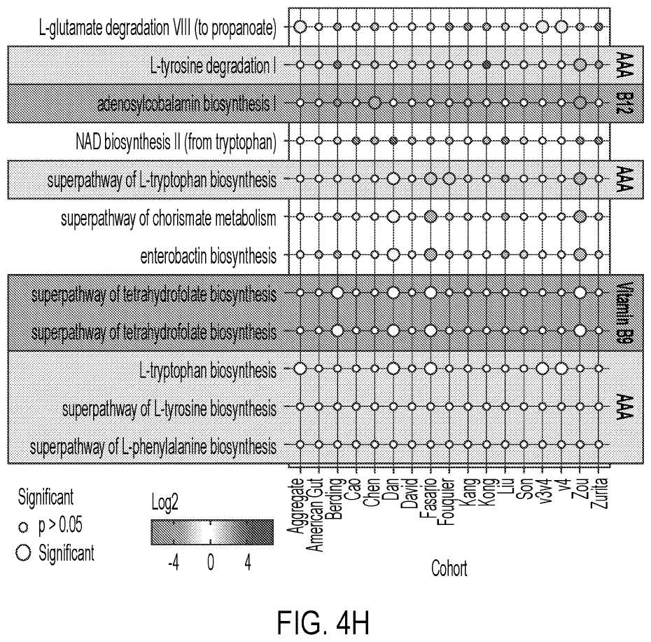

Differences in microbial relative abundances between ASD and control subjects were assessed at a per-study level, aggregating the data at different phylogenetic levels. Few phyla were consistently significantly different; the most commonly significant phylum was Bacteroidetes, with four studies reaching statistical significance (FIG. 4A). Despite not reaching statistical significance, a majority of cohorts did show increased Actinobacteria and Proteobacteria (FIG. 4A). The ratios of specific phyla were also investigated. It was found that the ratio of Firmicutes to Bacteroidetes was elevated in ASD individuals in Berding et al., 2020, Cao et al., 2021, Dan et al., 2020, David et al., 2021, Huang et al., 2021, Fouquier et al., 2021, Kong et al., 2019, Liu et al., 2019, and Son et al., 2015 (FIG. 4A). In contrast, the ratio of Firmicutes to Bacteroidetes was decreased in Zou et al., 2021, Chen et al., 2020, and Kang et al., 2017 (FIG. 4A). A significant difference in this ratio was not found between samples in the USA and China.

The most common genus to have a significant difference in its relative abundance was Prevotella, for which five cohorts reached statistical significance (unadjusted P<0.05) (FIG. 4B). However, studies were split as to whether Prevotella was increased or decreased in ASD subjects (FIG. 4B). Common ASD associations were also inconsistently increased or decreased at the class and order level, though most studies showed an increased abundance of the class Enterobacteriales among ASD subjects.

When analyzing the data in aggregate, there were also few taxa that generalized to the whole dataset. A few notable exceptions include the class Actinobacteria, which was significantly higher in ASD children compared to controls (Wilcoxon t-test P=0.0014, FIG. 4C). Despite inconsistent per-study findings for Prevotella, the general trend was found to indicate a decrease in ASD samples compared to controls (Wilcoxon t-test, P=0.044, FIG. 4D). One of the strongest findings among the aggregate data confirmed past associations with increased Bifidobacterium among ASD subjects (Wilcoxon t-test, P=0.00015, FIG. 4E).

### Chorismate Links Several Microbial Pathways Commonly Altered in ASD

Despite the taxonomic inconsistency across studies, functional pathways encoded by the microbiome of ASD subjects may be more generalizable. To test this possibility, the metabolic pathway abundances of each sample were evaluated and compared for ASD and control subjects. After assessing the most commonly significant pathways, two major clustering patterns of pathways were observed that were generally increased versus generally decreased in ASD subjects (FIG. 4F). Of particular interest, several of the commonly identified pathways shared a common molecular precursor, chorismate.

Chorismate is a product of the shikimate pathway, which is absent in humans. Chorismate can be transformed into several important molecules, including menaquinols and ubiquinols. 18 pathways involving the biosynthesis of various menaquinols and ubiquinols that were significant in three or more studies were identified (FIG. 4G). These pathways included the biosynthesis of menaquinols 6-13, which were largely increased in ASD apart from Zou et al., which showed a significant decrease among ASD subjects (FIG. 4G).

Menaquinones and ubiquinones are vital molecules involved in bacterial anaerobic and aerobic respiration respectively and can be synthesized from chorismate.

Aromatic amino acids (AAA's) are also products originating from chorismate. From the analysis, L-tryptophan biosynthesis was significantly altered (P<0.05) in four studies and increased among ASD subjects in nine of the fourteen studies (FIG. 4F). However, pathways related to the other aromatic amino acids, L-tyrosine and phenylalanine, were not as commonly significant, though L-tyrosine degradation was increased in ASD within eight of the fourteen cohorts (FIG. 4H). It was also noticed that other amino acid-related pathways, such as L-threonine biosynthesis and L-glutamate and L-glutamine biosynthesis, were significantly decreased in five cohorts each (FIG. 4F). These results support previously noted differences in amino acid metabolism in ASD patients and expand the analysis to new cohorts.

Other molecules derived from chorismate include siderophores and folate, which were also found to be altered among several ASD studies. Enterobactin is a metal chelating siderophore, which was identified to be significantly altered in Dan et al., Huang et al., and Zou et al., (FIG. 4H). Tetrahydrofolate biosynthesis was further identified to be significantly decreased in 4 studies (FIG. 4H). Further, adenosylcobalamin biosynthesis (also known as Vitamin B12) was decreased in ten of the fourteen cohorts, with four cohorts reaching concordant significance (FIG. 4F). Vitamin B12 catalyzes the conversion of homocysteine to methionine and tetrahydrofolate, playing a rate-limiting step on folate levels.

### Evaluating Machine Learning Performance on ASD Status Prediction

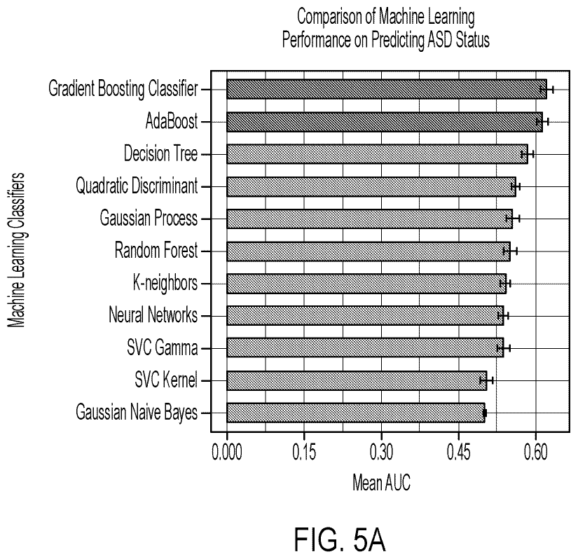

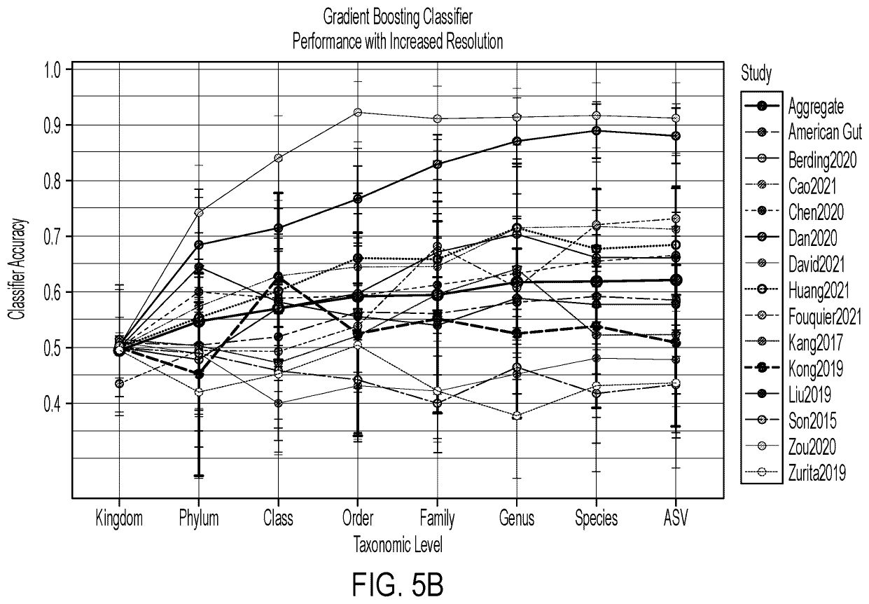

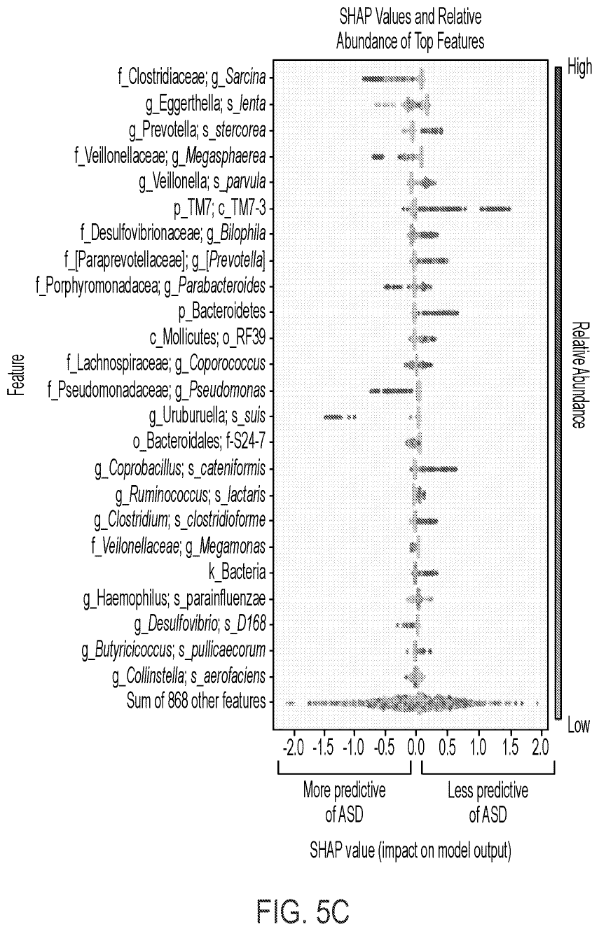

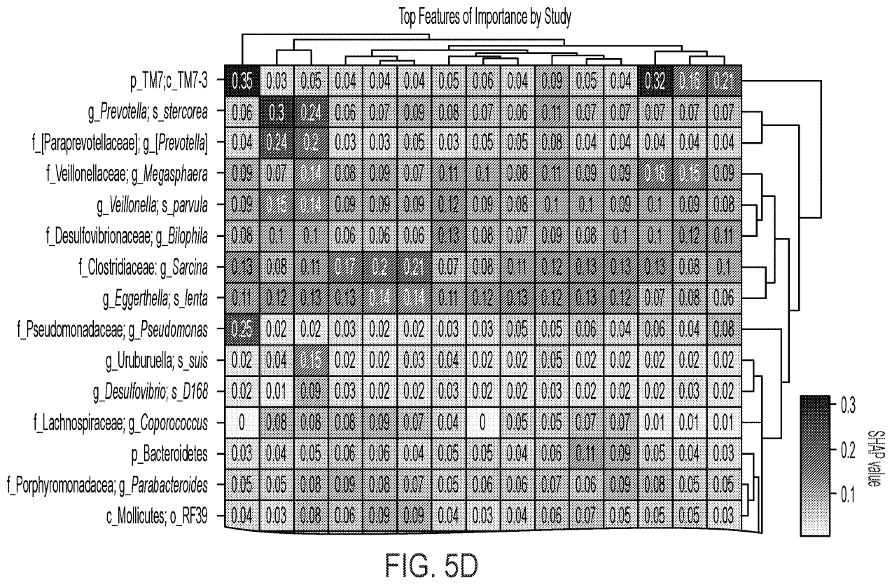

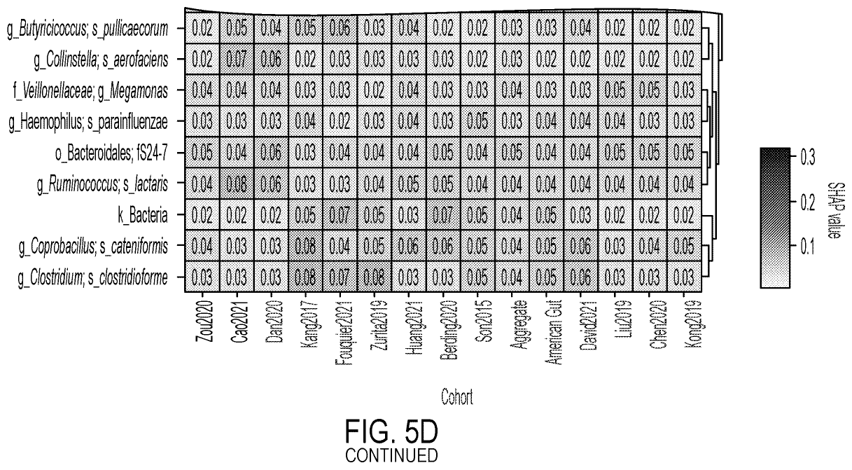

The relationship between the microbiome and ASD may be non-linear and unlikely to be identified through reductionistic approaches. Therefore, various machine learning approaches were utilized to assess whether the ASD gut microbiome is distinguishable from that of neurotypical individuals. The performance and accuracy of predicting ASD status were evaluated using eleven different classification algorithms across the aggregate of the fourteen datasets (FIG. 5A). All eleven models performed at least marginally better than chance as determined by having an area under the receiver operating characteristic curve greater than 0.5 (FIG. 5A). The top performing models, gradient boosting classifier (GBC) and adaboost classifier (ABC), were able to distinguish ASD individuals from controls with an area under the curve (AUC) greater than 0.6 (GBC; AUC=0.62±0.03, ABC; AUC=0.61±0.02) (FIG. 5A). Using the F1 score of each model to assess both precision and recall, it was found that GBC and ABC models performed at 0.62±0.03, and 0.62±0.02 respectively. The performance of each model was assessed by evaluating each model's classification accuracy. Both the GBC and ABC models reported the largest percent accuracy of 62%±0.03 and 61%±0.02 respectively. Across all the metrics, the GBC performed the best at classifying ASD status and was therefore selected for downstream analysis.

As the initial machine learning model comparison was performed on amplicon sequencing variants (ASVs), seeing how taxonomic resolution impacted the model's performance and accuracy was of interest. Fifty iterations of randomized train-test splits utilizing the GBC model on read count data either uncollapsed or aggregated at seven different taxonomic levels (Kingdom, Phylum, Class, Order, Family, Genus, Species, ASV) were performed. Increasing taxonomic resolution improved the model's accuracy, with only marginal gains past genus level classification (FIG. 5B). Similar trends were observed when performing the same analysis with both the adaboost and decision tree classifiers. Training and testing each dataset individually, GBC models performed better than chance (AUC>0.5) in all but three data sets, and five had AUCs above 0.70 (FIG. 5B). These results indicate that the ASD gut microbiome may contain information that would be beneficial to assessing ASD status.

The important features in distinguishing individuals with ASD from controls were next investigated. The top three features from the GBC model trained and tested on the aggregated ASV-level dataset were an ASV from the genus Sarcina (family Clostridiaceae), Eggerthella lenta, and Prevotella stercorea (FIG. 5C). Among the top 25 features, increased relative abundances of an ASV from Sarcina, the genus Pseudomonas, and Uruburuella suis were important for ASD classification, while increased abundances of an ASV from class TM7-3, the phylum Bacteroidetes, Coporobacillus cateniformis, and Clostridium clostridioforme were important for control classification (FIG. 5C). To provide more context to the findings, the top 25 features from the aggregate dataset were further assessed for their importance to predicting ASD status within GBC models generated on each individual dataset. Hierarchical clustering of the importance scores of these features to each data set revealed two main groupings (FIG. 5D). Of particular interest was a group of features that contained similarly high importance scores across all datasets. This group included an ASV from Eggerthella lenta, the genus Sarcina, the genus Bilophila, Veilonella parvula, and the genus Megasphaera. These top features highlight that despite the heterogeneity of results among studies, there are several microbial features that can consistently be utilized for ASD classification.

### Evaluating the Influence of Study Design on Study Outcomes

To address the variability in ASD microbiome results reported in the literature and highlighted in this Example, it was sought to evaluate how factors of study design can impact the findings of an ASD microbiome study. Some of these factors include, but are not limited to, 16S sequencing methods, geographic location, sex, age, and the relationship of the control subject to the ASD subjects. Regarding various technical choices of 16S sequencing methods, the effect of sequencing depth and choice of 16S hypervariable region were evaluated.

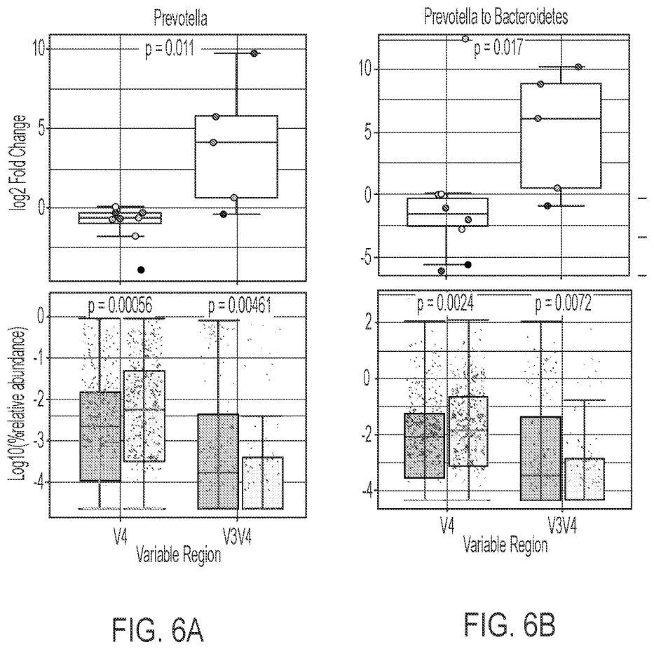

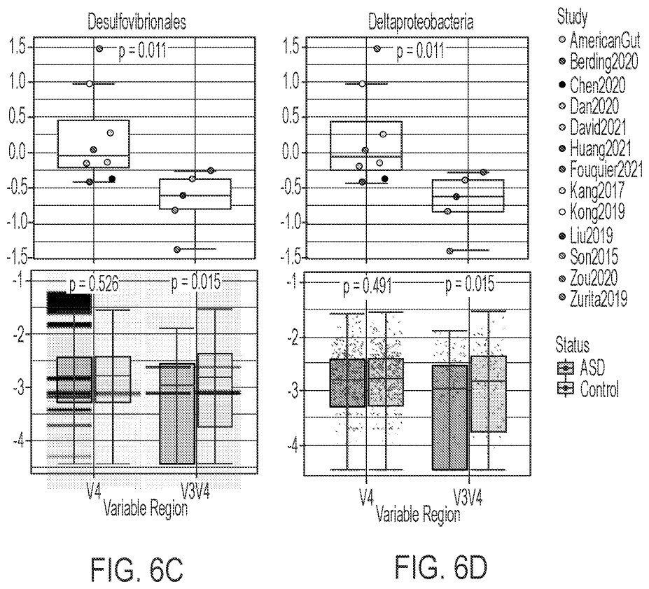

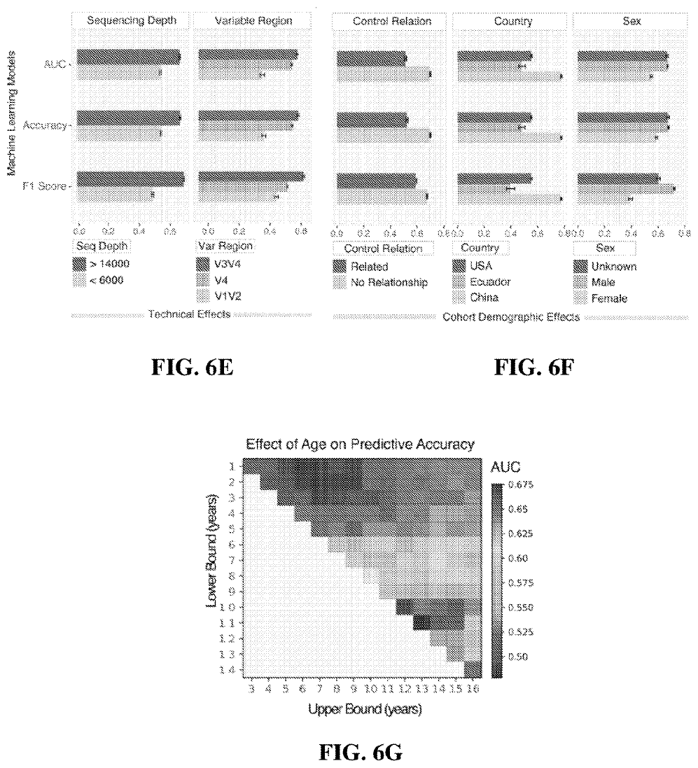

Among the 16S hypervariable regions included in the data, one study targeted the V1-V2 region, 5 studies utilized the V3-V4 region, and 8 studies used the V4 region. V1-V2 samples were excluded from this analysis given the limited number of studies conducted utilizing that variable region. It was found that the studies utilizing the V3-V4 variable region had a significantly higher relative abundance of the genus Prevotella in ASD subjects (P=4.61E-3) while studies targeting the V4 region had a significantly lower relative abundance of this genus in ASD subjects (P=5.6E-4) (FIG. 6A). This was also consistent when comparing the ratio of ASD/control relative abundances for Prevotella from a per-study perspective (P=0.011) (FIG. 6A). It was also found that the ratio of the genera Prevotella to phylum Bacteroidetes, the order Desulfovibrionales, and the class Deltaproteobacteria were different between the V3-V4 and V4 regions (per-study Wilcoxon t-test P=0.017, 0.011, and 0.011 respectively) (FIGS. 6B-6D). When analyzing the data in aggregate as opposed to on a per-study basis, it was noted that significant differences could be found for Desulfovibrionales and Deltaproteobacteria among V3-V4 studies, while no difference was found among V4 samples (both decreased in V3-V4 ASD with a P=0.015).

Despite identifying discrepancies between taxonomic associations to ASD by sequenced variable region, machine learning models trained on both data sets performed well above chance while V3-V4 data outperformed V4 data (average AUC of 0.65 and 0.70 for V4 and V3-V4 models respectively) (FIG. 6E). These results suggest that there are notable taxonomic differences that may occur dependent on the 16S hypervariable region sequenced, however machine learning models built for ASD classification are possible when utilizing either the V4 or V3-V4 hypervariable region.

A split in the dataset was noted in regards to sequencing depth, where 6 studies were sequenced at a relatively lower sequencing depth (sequencing around 6,000 reads per sample) and 8 were sequenced at a higher sequencing depth (sequencing a minimum of 14,000 reads per sample). There was a notable difference in the performance of the model trained on samples from cohorts with a minimum of 14,000 reads per sample, to that of the cohorts with a minimum of 6,000, as assessed by the AUC, Accuracy, and F1 statistic (FIG. 6E). Models trained on data from studies using a high sequencing depth had an average AUC of 0.75 while studies with a low sequencing depth had an average AUC of 0.59. However, from a taxonomic perspective, no significant effect of sequencing depth was found on the relative abundance differences of ASD to controls. These results suggest that increased sequencing depth may be important for accurate ASD classification, while not impacting taxonomic results.

Next, it was evaluated how aspects related to the demographics of the study participants may alter ASD microbiome results. For the effect of geographic location, the cohorts were split by which country each study's subjects were from. No statistically significant differences in ASD to control relative abundance ratios from studies in the USA versus studies from China were found. However, a striking difference was found in the ability of GBC models to classify ASD and control samples dependent on country (FIG. 6F). It was found that the models performed better on Chinese samples, with an average AUC of 0.78 versus an AUC of 0.6 on samples from the USA. These results suggest that geographic location may be an important factor for ASD classification utilizing the gut microbiome.

Some studies utilized age and sex match controls, while other studies used household controls such as siblings or mothers. No statistically significant differences in ASD to control relative abundance ratios from those with related individuals as controls to those with unrelated individuals were found. A notable difference was observed in the GBC model's ability to classify ASD and controls from the cohorts which utilized unrelated controls compared to those with related controls (FIG. 6F). The model performed better from samples of cohorts with unrelated controls, with an average AUC of 0.71, compared to cohorts with related controls, which had an average AUC of 0.54. This indicates that there may be larger differences in the microbial profile of ASD individuals compared to controls when the controls are not related to the ASD individual.

It was also assessed how sex influenced microbial profiling and the diagnostic accuracy of the machine learning model of ASD and control individuals. The samples were split by sex and the AUC of the model for predicting ASD status of females was compared to males (FIG. 6F). A greater accuracy for predicting ASD status from male samples was observed, with an average AUC of 0.67 versus an AUC of 0.55 from female samples (FIG. 6F). Taxonomic differences of ASD vs. controls on a per-study basis were not examined due to the limited number of females within each dataset. However, the difference in predictive accuracy of ASD status from male samples compared to that of females may indicate the influence of sex on microbial profiling and subsequent predictive accuracy.

The microbial composition of the gut microbiome is known to shift during human development. It was thus assessed how the age of the samples influenced the ability to predict ASD status. To do this, the data was subset at every potential age range between the ages of 1 and 16 and the performance of GBC models in predicting ASD status was compared (FIG. 6G). It was observed that samples from the ages of 2-7 years old obtained the greatest predictive accuracy and that as the age of the individuals increased, the AUC of the model concurrently decreased (FIG. 6G). To validate that the findings were not simply due to the corresponding sample sizes available at each age range, the effect of sample size on GBC model performance was first determined, and the corresponding sample sizes were then plotted. It was observed that samples from younger subjects performed better than expected by sample size at ASD status prediction.

### Chorismate Metabolism Branch Point Enzymes

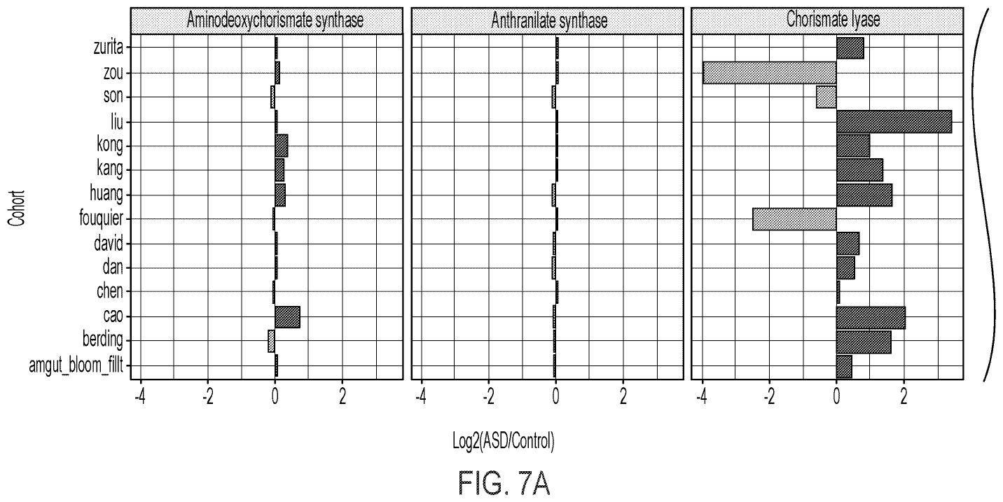

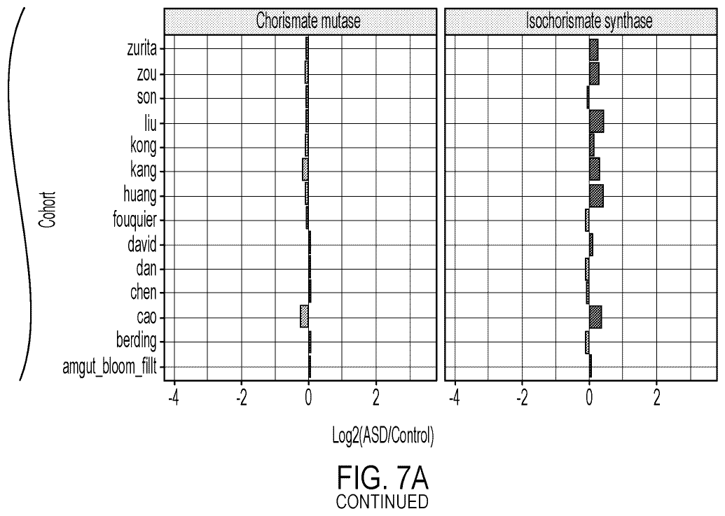

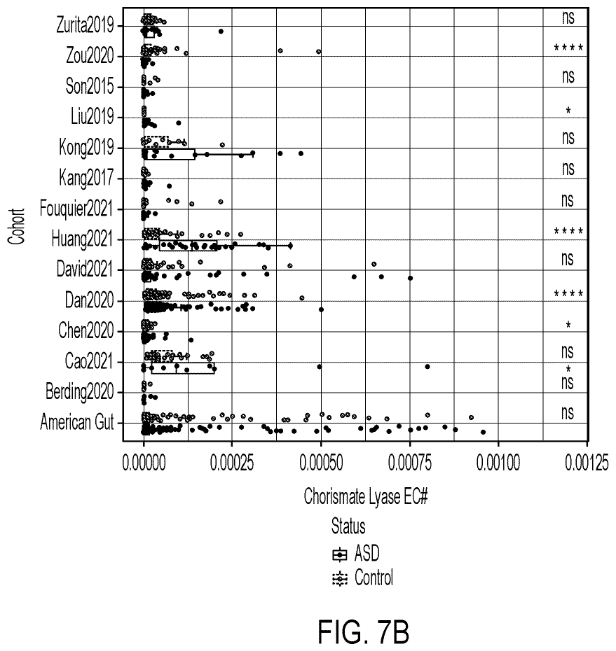

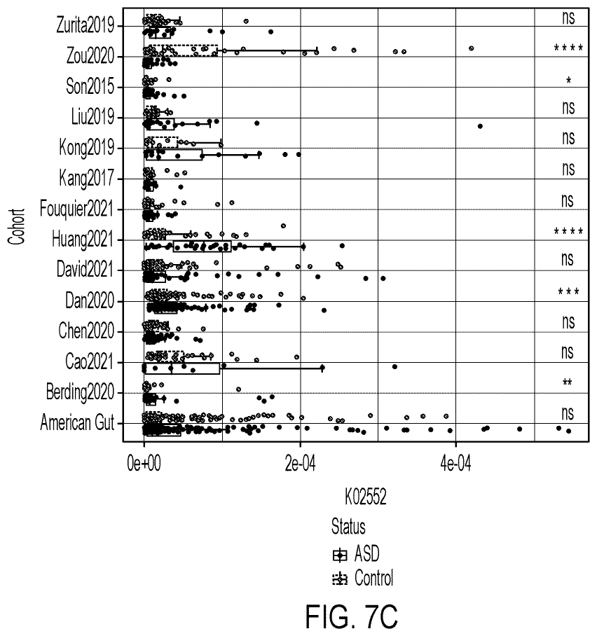

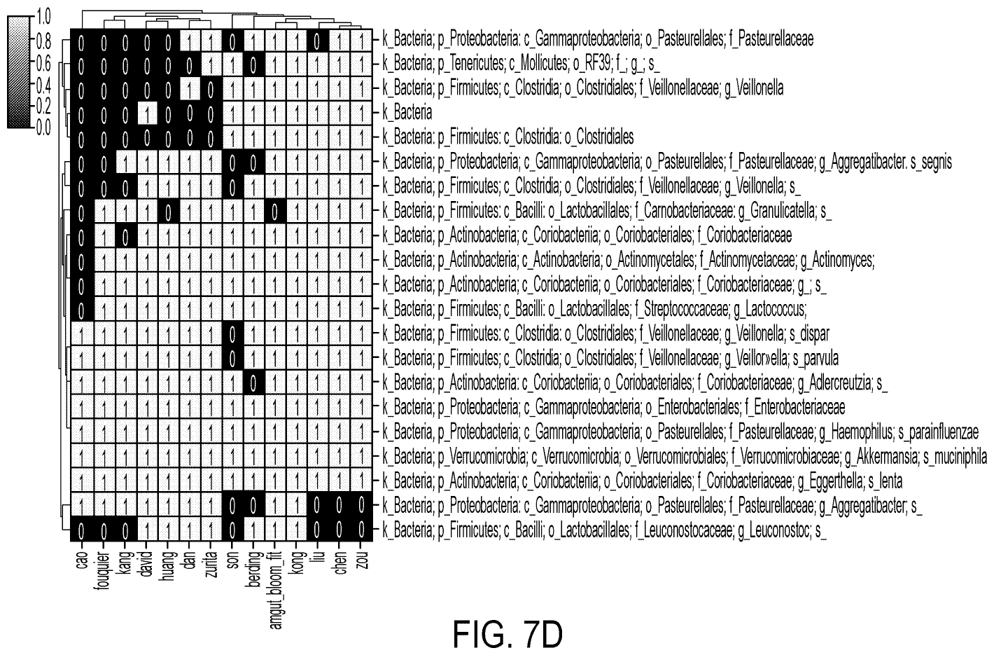

In addition to assessing the differences in the pathways derived from chorismate listed above, it was assessed whether the six branch point enzymes of chorismate metabolism were altered in the ASD gut microbiome. It was observed that the enzyme chorismate lyase, which commits chorismate down the path of ubiquinol biosynthesis, was upregulated in the ASD gut microbiome in eleven of the fourteen cohorts, for which two reached statistical significance (FIG. 7A). It was observed that there was a much larger difference in ASD gut microbiome compared to controls for the branchpoint enzyme chorismate lyase than any of the other branch point enzymes. To see if ASD samples have a greater number of chorismate lyase enzymes than their control counter plots, the normalized enzyme number was calculated for each sample, and the numbers for ASD individuals and controls were compared for each study (FIG. 7B). It was shown that five cohorts showed statistically significant differences in the predicted number of chorismate lyase enzymes in the ASD gut microbiome (FIG. 7B).

Lastly, one of the most commonly significant pathways related to chorismate was the biosynthesis of various menaquinols. It was desired to investigate whether this might be due to an increase, or decrease, in the branchpoint enzyme responsible for committing chorismate to the production of menaquinols, which is menaquinol-specific isochorismate synthase. Isochorismate synthases have been documented to convert chorismate to both enterobactin biosynthesis and menaquinol biosynthesis, and are classified under the same enzyme commission number (EC 5.4.4.2). The isochorismate synthase, menF, which is found in several bacterial species, and which commits chorismate to menaquinol biosynthesis, was further investigated. The predicted gene count for menF was examined, and it was assessed whether it was more or less enriched in ASD fecal samples compared to controls. Three studies showed statically significant differences in the predicted gene abundance for menF (FIG. 7C). While not reaching significance, a majority of studies showed an increase in the menaquinol specific isochorismate synthase in the ASD gut microbiome (FIG. 7C). This trend is comparable to that observed for the entire menaquinol biosynthesis pathways mentioned previously (FIG. 4H).

The next goal was to identify which bacteria contained the menF gene. The bacteria that contained the menF gene in each cohort were identified. A heatmap of the bacteria was generated to assess how prevalent they were in each cohort (FIG. 7D). Four of the bacteria that harbored the menF gene were of the bacteria important for ASD of control classification from the machine learning model (FIG. 5C). The increased importance of these bacteria for the machine learning classification models elucidated further that chorismate, chorismate-related metabolites, or chorismate converting enzymes are important in the ASD gut microbiome and may make promising therapeutic targets.

### Discussion of Results

In this example, a systematic approach to reanalyzing the existing ASD gut microbiome data and providing an in-depth report of the commonalities and discrepancies between datasets was taken. The extent to which aspects of study design influence the taxonomic findings and accuracy of machine learning models designed for ASD classification was tested. Given the variety of choices in different data processing approaches, and the variability these choices introduce, it was important to reanalyze the data using consistent methodology. With the data set, common questions regarding how the ASD microbiome differs from control individuals addressing alpha-diversity, beta-diversity, and relative abundance differences of the microbiome at different taxonomic levels were revisited. Mixed results were found in whether alpha-diversity differences were observed, with slightly more consistency in beta-diversity differences of ASD individuals compared to controls. What is commonly reported was expanded by analyzing the data based on factors such as variable region, control relationship, age, sex, and geographic location and by utilizing machine learning models to estimate the impact of these factors on the classification of ASD status using microbiome data.

This Example contextualizes the ASD microbiome field by evaluating how both technical choices and cohort demographics alter results. Microbial compositions are known to vary across different human populations and based on technical choices. Of note, significant discrepancies in the diagnostic capability of the machine learning models based on data from the USA vs. data from Chinese cohorts were observed. One possible explanation is the collection of more severe ASD cohorts in China than the USA. Supporting this is a report that suggests that the prevalence of ASD in China may be underestimated and that the true prevalence rates may be closer to those in the USA. This may indicate that children with an ASD diagnosis in China may represent more severe cases than their western counterparts. Alternatively, the results may also be a result of different countries representing populations that differ in socio-economic status, diets, living conditions, and environmental factors.

The increased ability to predict ASD status at a younger age was also found to be of particular interest. This suggests that developing a diagnostic screening tool based on the microbiome could facilitate early diagnoses, which may be of great interest given the limited diagnostic tools available for ASD to date. It is also possible that these results represent a critical window of ages in the pathogenesis of ASD. However, many confounders exist, including bias in the studies that samples belonged to or the potential that ASD cases at ages 2-7 represent more severe cases than those of older children.

Regarding study discrepancies in taxonomic associations to ASD, the most important factor was found to be the choice of 16S hypervariable region. Discordant results were found in both the per-study and per-sample relative abundance for the ratio of Prevotella to Bacteroidetes, genus Prevotella, order Desulfovibrionales, and class Deltaproteobacteria in ASD vs. controls in the V3-V4 cohorts to the V4 cohorts. It was particularly interesting that Prevotella appeared to be increased in ASD among V4 datasets while it was decreased in ASD among V3-V4 cohorts. The V3-V4 region represents a larger portion of DNA (˜300 bp vs. ˜150 bp among these studies), so one potential explanation for this could be varying taxonomic assignments of reads based on longer or shorter read lengths.

Despite large study variability, several consistent trends in the ASD gut microbiome were identified across the cohorts. The largest taxonomic consistency was the enrichment of Actinobacteria, at both the phylum and class level, as well as increased Bifidobacterium, and a general decrease in Prevotella among ASD individuals. The genus Bifidobacterium (phylum Actinobacteria) is important to human development early in life, while it is later taken over by strict anaerobic bacteria. In some cases, the increased prevalence of Bifidobacterium may be indicative of an underdeveloped gut microbiome.

Some consistencies among the predicted functional pathways encoded by the ASD gut microbiome were also found. Chorismate is a common branch point for the synthesis of several biologically relevant molecules, including the AAA's, menaquinones, ubiquinones, enterobactin, and folate. Several pathways related to these downstream metabolites were found to be altered in the ASD gut microbiome. Intriguingly, chorismate biosynthesis is dependent on the enzyme 5-enolpyruvylshikimate-3-phosphate synthase (EPSPS), which is the target of glyphosate. In some cases, glyphosate levels may be linked to ASD rates and an altered microbiome.

### Methods

### Literature Search Strategy and Selection Criteria

A systematic literature search was performed through the PubMed database, from inception to Jul. 15, 2021, to identify applicable studies assessing autism spectrum disorder and the gut microbiome. The following search terms were used as free-text words only, or as key words: ASD (or Autism Spectrum Disorder or Autism) and (gut microbiome, or microbiota, or fecal microbiome). No other search features or advanced language limits were used. Articles were included in this meta-analysis if: (a) fecal microbiota samples were taken of a patient with autism and/or that of a typically developing individual; (b) data and respective metadata were retrievable and interpretable; and (c) a autism diagnosis was given by a healthcare professional. From the literature search, a total of 244 studies were assessed for inclusion. Of the 244 studies, 36 studies indicated cohorts wherein the microbiome's role in ASD was researched and were read in full to assess their inclusion eligibility. A total of 12 studies met the inclusion criteria described above and were included in this analysis. One dataset deposited in the Sequence Read Archive (SRA) with its respective metadata, that was not yet published but published during manuscript preparation, was identified. Lastly, open-source data generated from the American Gut Project (AGP) was utilized to cultivate an additional dataset of ASD children and controls. A propensity score algorithm was designed to identify a best-match control subject for each ASD subject. Together, these 14 cohorts yielded a total of 888 ASD samples and 852 control samples of typically developing individuals from a wide range of ages and geographic locations.

### Exclusion Criteria

With the goal of minimizing known technical effects, studies which only utilized shotgun metagenomic sequencing methods, Roche 454 sequencing, and/or 16S rRNA pyrosequencing were not included. Thus, 15 studies were excluded from the analysis for incompatible sequencing methods. Eight other studies met the inclusion criteria but did not have publicly available metadata. All eight authors were contacted but did not respond to a request for the metadata and thus were unable to be included in the analysis.

Compiling a Cohort from the American Gut Project

The AGP dataset contains samples from more than 15,000 participants, thus representing a diverse dataset to compile targeted cohorts with. All available 16S data and metadata were downloaded from Qiita.ucsd.edu (https://giita.ucsd.edu/study/description/10317). Fecal samples from subjects who were diagnosed with ASD by a physician were first identified, and a control cohort based on each ASD subject's features was compiled. Therefore, a method based on propensity score matching was adapted which consisted of two main steps: scoring and matching. For each sample in the ASD cohort, a corresponding control sample was selected based on having an exact match of specific metadata features (run date, sample type, and sex) and having the closest “similarity score” calculated from other metadata features (age, BMI, c-section, country, diabetes, antibiotic history, probiotic frequency, prepared meals frequency, allergies, prepared methods frequency, red meat frequency, fermented food consumption, whole grain frequency, vitamin B supplementation frequency, plant protein frequency, vitamin D frequency, vegetable frequency, epilepsy or seizure disorder). Exact matching allowed selection of features to be controlled for. For example, to control for technical variability between sequencing runs, it was required for the control sample to have come from the same sequencing run date as the ASD sample. On the other hand, the similarity scores were simply the conditional probability of cohort assignment given a vector of metadata features. In this way, the matched control samples represented a subset of the control cohort that had similar, if not the same, metadata feature distributions as that of the ASD samples.

To calculate similarity scores, each feature was first binarized using the OneHotEncoder class from scikit-learn version 0.24.2 (https://scikit-learn.org/). These features were then fit using scikit-learn's LogisticRegression class where the dependent variable was cohort status (i.e. 1 for ASD cohort; 0 for control cohort). The similarity score was the output of the sigmoid function, which was also the predicted probability of being assigned to the ASD cohort.

During the matching process, the similarity score from each sample in the ASD cohort was compared with the similarity score from every control sample whose “exact matches” variables were the same. The control sample with the closest similarity score was then placed in the control cohort and removed as a possible control sample for subsequent rounds of matching. After matching every ASD sample, a control cohort of unique samples and whose sample size was the same as that of the ASD cohort was obtained.

### Data Acquisition

For data publicly deposited in the NCBI Sequence Read Archive (SRA), sample metadata was downloaded from each project using the SRA Run Selector tool (https://www.ncbi.nlm.nih.gov/Traces). A custom script (process_experiment.py) available on github (https://github.com/mortonjt/GetData) was used to systematically download, trim primers and process samples into data tables. In cases where data and sample metadata were not deposited through SRA, they were provided directly from the original study's authors and processed using identical parameters as described below.

### Standardized 16S Processing Pipeline

One goal was to collect and reprocess each study in a systematic and harmonious way to reduce errors imposed by different processing methods and enable the drawing of better conclusions about the ASD gut microbiome and the dataset as a whole. Each study was processed individually utilizing the same processing steps with the QIIME 2 (v. 2020.8) software. All the samples were also processed together with the same pipeline to draw conclusions and uncover trends about the aggregated data as a whole. The first step of the systematic processing pipeline was the demultiplexing and removal of primers when needed, utilizing the custom script process_experiment.py. Next, all sequences were trimmed to a length of 150 bp. After trimming, sequences were denoised and filtered using the Deblur QUIME 2 2020.8 plugin. Taxonomic classification was performed with the QIIME 2 q2-feature-classifier plug-in with the full length 16S pre-fitted GreenGenes-trained Naive Bayes classifier (gg-13-8-99-nb-classifier.qza) provided by QIIME 2 (QIIME 2 v. 2020.8). Phylogenetic placement was assigned with the SEPP fragment insertion plug-in from QIIME 2 (v. 2020.8). SEPP was chosen to minimize the effects of hypervariable regions by assigning short reads to a reference phylogeny. All features that were not aligned to the tree were removed with the q2-fragment-insetion filter-features QIIME 2 2020.8 plugin. The resulting tables were ASV-level read counts which were used for all downstream analyses.

Prior to alpha and beta diversity analyses, the sequences from each cohort were rarefied independently to the necessary depth to retain the maximum number of reads as possible, while filtering any samples where the number of reads was very low. For the aggregated data, the resulting table was rarefied to a sequencing depth of 6000 with the core-phylogenetic QIIME 2 v. 2020.8 plug-in. Alpha diversity was assessed with Faith's phylogenetic diversity, Shannon diversity, species evenness, and observed features from the core-phylogenetic QIIME 2 v. 2020.8 plug-in. The unweighted UniFrac and weighted UniFrac distance matrices, also calculated using the core-phylogenetic QIIME 2 v. 2020.8 plug-in, were used to compute beta diversity distances and corresponding Principle Coordinates Analysis (PCoA) plots.

### Analysis of Alpha-Diversity, Beta-Diversity, Taxonomic and Functional Differences

All samples that contained less than 6,000 reads per sample were filtered before all fold change, relative abundance, pathways abundance, and machine learning comparative analyses, for both the aggregated data and per-study level analysis. Analyses were performed using custom Python (version 3.6.0) scripts. Statistical significance of beta-diversity distances between groups was calculated using the QIIME 2 beta-group-significance and reported p-values were calculated using Permutational multivariate analysis of variance (PERMANOVA). Differences between ASD and control subject's alpha-diversity were performed only on a per-cohort basis due to large variability between studies. The alpha diversity log 2 ratio of ASD to controls was performed in NumPy version 1.19.1 (https://numpy.org/), from ASD and control subjects mean values as calculated using SciPy version 1.5.2 (https://scipy.org/). The significance of the log 2 fold change for each cohort was assessed by an unadjusted, two-tailed t-tests with assumed unequal variance in SciPy version 1.5.2.

For the per-study log 2 fold changes of each taxonomic level mentioned, samples were first normalized to relative abundances by dividing ASV read counts by the total reads per sample. Relative abundances were summed at each taxonomic level using Pandas version 0.25.3 (https://pandas.pydata.org/). At the per-study level, log 2 ratios of ASD and control subject means were calculated using SciPy version 1.5.2 and log transformed in NumPy version 1.19.1. Statistical significance was assessed in the same manner as that for alpha diversity described above. The log 10 transformed relative abundance in each sample for the significant taxa that were highlighted was performed in Pandas version 0.25.3. To avoid instances of infinity due to log 10 transformation, the minimum non-zero normalized relative abundance value was added to all samples utilizing NumPy version 1.19.1. Next, each sample's normalized relative abundance was log 10 transformed with NumPy version 1.19.1 before plotting.

Microbial functional profiles were estimated from 16S data using the Phylogenetic Investigation of Communities by Reconstruction of Unobserved States (Picrust2) software (v. 2019.1). The resulting pathway abundance tables were analyzed for differences between ASD and control subjects as described above. All figures were generated using ggplot2 version 3.3.3 in R version 4.0.5.

### Machine Learning Classification of ASD Status

To predict ASD status from the microbiome, eleven different classification models, evaluation metrics, and auxiliary functions from the scikit-learn package version 0.24.2 (https://scikit-learn.org/) were used. Unless stated otherwise, 5-fold cross validation repeated 10 times was used to estimate the averages and variances of different performance metrics. Hyperparameter optimization was not performed. Reported results included the area under the curve (AUC) of the ROC curve, as well as the accuracy and the F1 score of each model.

During the initial evaluation of different machine learning models, the aggregate ASV-level dataset was used and normalized so that relative abundances summed to unity before training and evaluating each machine learning model. Next, for each study (including the aggregate dataset), the dataset was collapsed at different levels of taxonomical hierarchy (e.g. genus) by grouping each taxa (e.g. Clostridium), summing their raw read counts, and normalizing so that the relative abundances summed to unity. This processed dataset was then used for training and testing the GBC, ABC, and the decision tree classifier from scikit-learn. Reported results include the area under the curve (AUC) of the ROC curve, as well as the accuracy and the F1 score of each model.

To determine the importance of each feature, the Shapley additive explanations (SHAP) package (https://github.com/slundberg/shap) was applied to the Gradient Boosting Classifier model trained on the aggregate ASV dataset. The top 25 most important features by cumulative SHAP value were displayed using the beeswarm plotting function of the SHAP package. Absolute SHAP values from each of the top 25 features were averaged for each study and displayed as a clustermap using the seaborn package version 0.11.0 (https://seaborn.pydata.org/). To determine the effects of different metadata variables, the entire dataset was separated by their corresponding metadata features. Each partition of the dataset was further split using 5-fold cross validation, and this data was used to train and evaluate a Gradient Boosting Classifier trained to predict ASD or control status. The AUC of ROC, F1 score, and accuracy were used to evaluate model performance. The entire process was repeated 50 times to estimate the standard error of each performance metric.

To estimate the predictability of ASD at different age ranges, the predictability of ASD across different age ranges from 1- to 16-year-olds was evaluated. That is, only the subset of data that fell into each age bin (e.g., 1-3 year olds) was used. To ensure sufficient data within each age bin, the lower and upper bounds of each bin had to be separated by 2 years. Using 5-fold cross validation of this subset of data, 30 instances of GBC models were trained and evaluated. The average AUC of the ROC curve of each age bin was displayed using a seaborn heatmap. Age ranges that were invalid (e.g., 3-1 year olds) were excluded and displayed as a gray square.

### Data Availability

Data utilized in this Example was from publicly available datasets deposited in the Sequencing Run Archive (SRA, https://www.ncbi.nlm.nih.gov/sra). Data can be accessed using the following study identifiers: PRJNA282013, PRJNA578223, PRJNA642975, PRJNA453621, PRJNA644763, PRJNA687773, PRJNA624252, PRJEB27306, PRJEB27306, PRJNA529598, PRJEB42687. Other datasets included data from David et al. 2021 (available at http://files.cgrb.oregonstate.edu/David Lab/ASD_study 1/) and data from the AGP available from Qiita under study identifier 10317 (https://qiita.ucsd.edu/study/description/10317).

### Code Availability

Data processing scripts are available on Github (https://github.com/mortonjt/GetData). Notebooks for both data analysis, and visualizations are also available on Github (https://github.com/erxw/ASD_16S_metaanalysis).

### Example 2

In this Example, further meta-analysis was conducted on additional cohorts. While the data set used in Example 1 consisted of 1,740 samples from 14 cohorts, the data set used in this Example consisted of 3,589 samples from 22 cohorts of 16S sequencing data on ASD and control subjects. A meta-analysis framework was established for identifying commonly altered taxa, the functional differences between gut microbiomes, and the predicted metabolic fluxes occurring in subjects' microbiota.

### Sarcina

Taxonomic analyses identified a potential biomarker of ASD status in the presence of sequencing data from the genus Sarcina. The work of this Example suggests around 14% of all ASD subjects contained a threshold abundance of the species while only 6.4% of control subjects contained a similar abundance of the species.

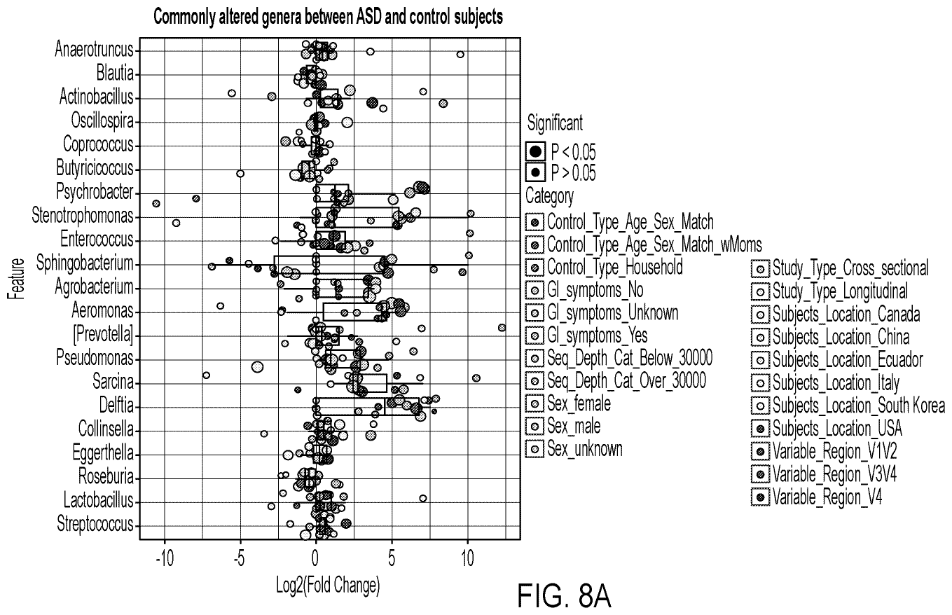

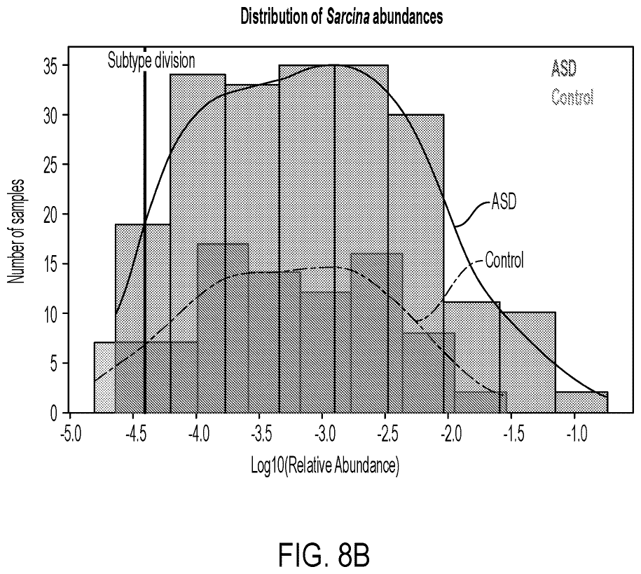

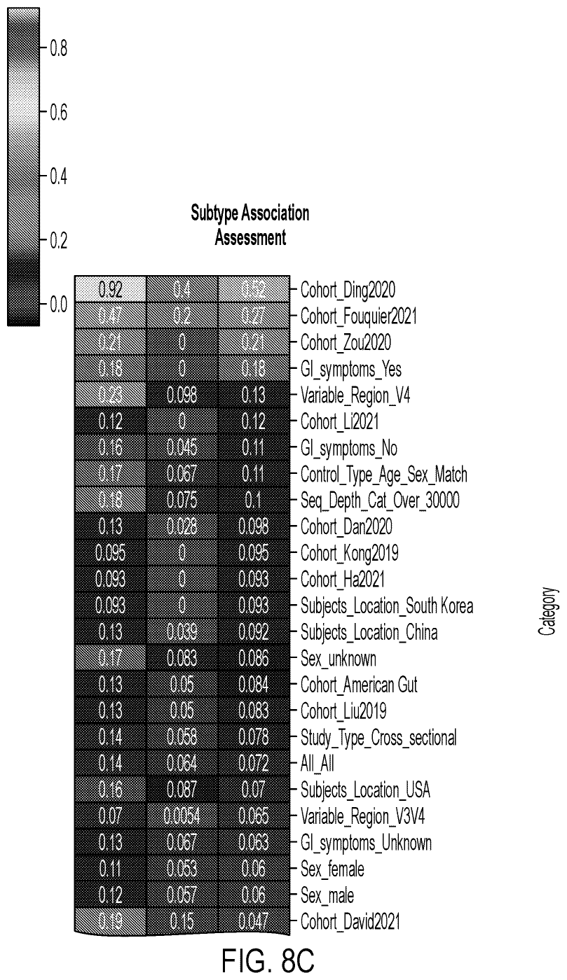

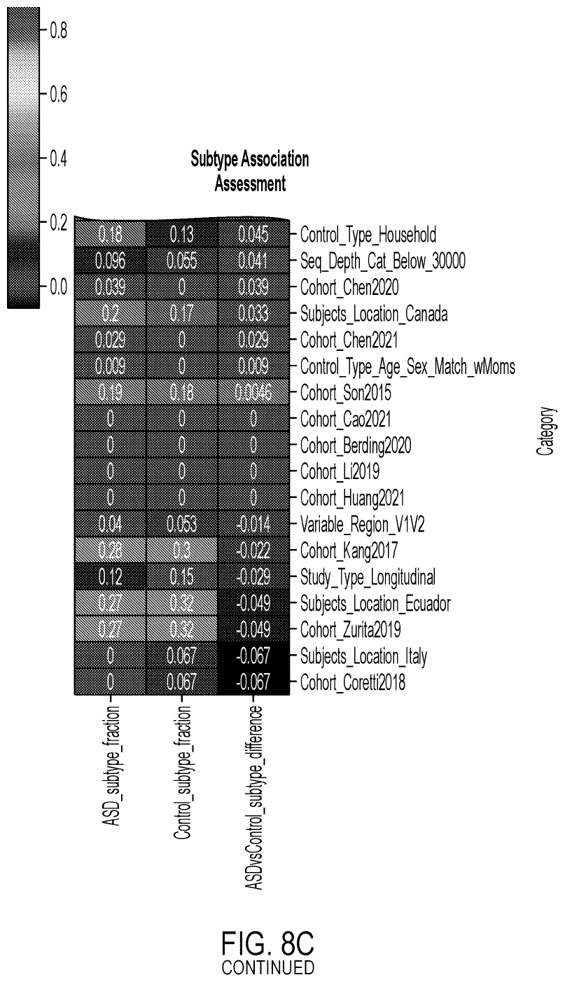

FIG. 8A shows commonly altered genera between ASD and control subjects. Compiled 16S sequencing data was collapsed at the genera level and split among different metadata characteristics regarding samples to compare differences between ASD and controls based on various factors. Log 2 transformed fold-change of the ASD/Control compositional comparison was plotted along each factor, and significance according to a ranksum test adjusted for multiple hypothesis testing was expressed by the size of each point. Boxplots displaying the interquartile range (IQR) show the general distribution of data. Notably, the IQR for Sarcina was entirely beyond 0, suggesting a biomarker of ASD status resilient to varying study factors.

The Sarcina biomarker may be Clostridium ventriculi or closely related to this species. Other closely related species include Clostridium perfringens, which is known to contain several toxins that can induce ASD-like symptoms, including tachycardia, dehydration, anorexia, GI problems, tremors, dullness, depression, lethargy, hyperesthesia, hyperactivity, irritability, seizures, neuroinflammation, permeable intestine/blood-brain barrier, and oligodendrocyte death. Therefore the toxin(s), or bacteria, may be a target for therapeutic interventions in ASD.

FIG. 8B shows histograms of compositional abundance (when present) of Sarcina in the gut microbiome for ASD and control subjects. FIG. 8B also shows overlaid kernel density estimation (KDE) curves for ASD and control subjects. An optimal discriminatory boundary for classifying ASD vs. controls was determined and plotted in FIG. 8B.

FIG. 8C is a heat map describing the fractional abundance of ASD or control subjects containing more than a threshold abundance of Sarcina in their gut microbiome, with the data set divided by cohorts and study factors. In addition, the difference between the ASD fractional abundance and control fractional abundance was plotted, showing the cohorts and study factors where this feature was highly enriched in ASD subjects.

### Chorismate

Meta-analysis of the expanded data set of this Example has largely supported the findings from Example 1 and bolstered the claims suggesting that chorismate is a key molecule, likely decreased in the gut of many ASD subjects. Upon reanalysis of the most commonly significant pathways, chorismate-related pathways were frequent.

The chorismate metabolism pathway has been identified as being present in most, if not all, gut microbiomes, and ASD subjects had a higher proportion of their microbiome devoted to these pathways. Identification of a discriminatory boundary based on chorismate metabolism identified cohorts and study factors where chorismate metabolism was particularly good at discriminating ASD and control subjects. These findings suggest that interventions based around chorismate may be particularly relevant in locations such as Italy, are non-discriminatory to the sex of ASD subjects, and may not be related to gastrointestinal symptoms.

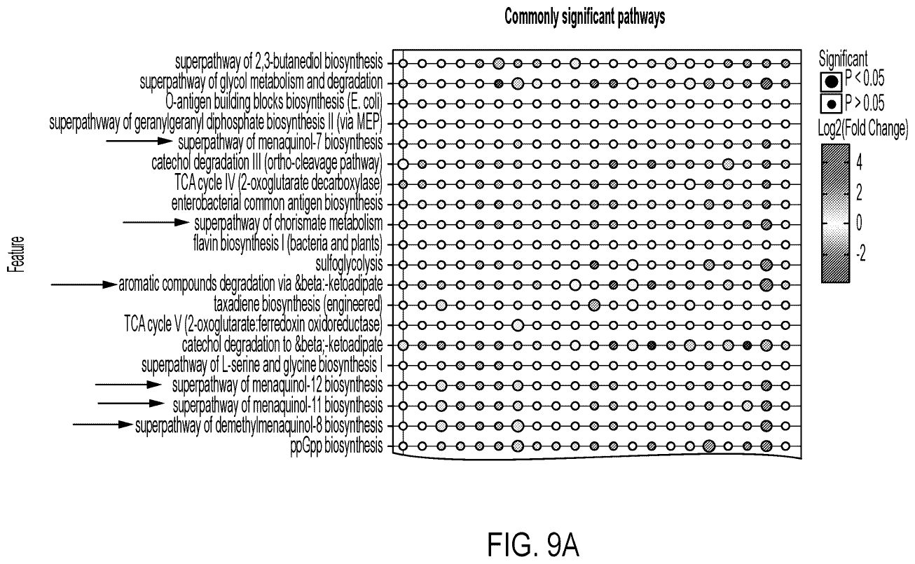

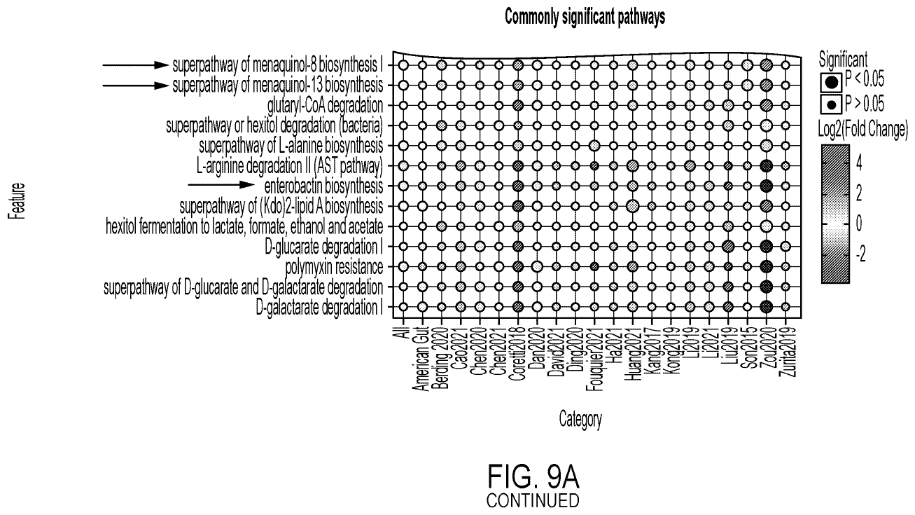

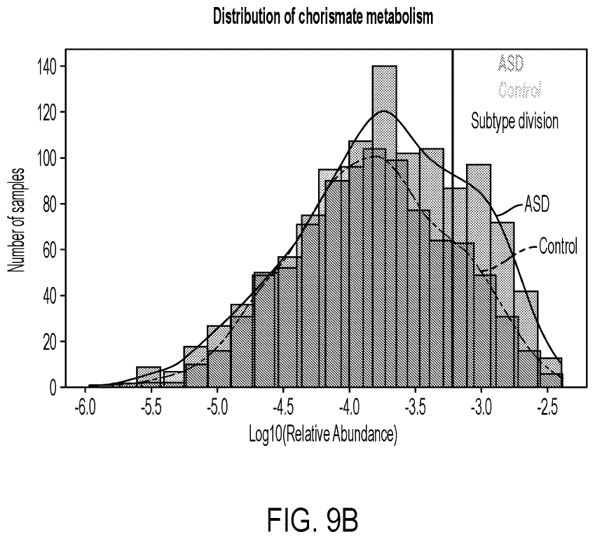

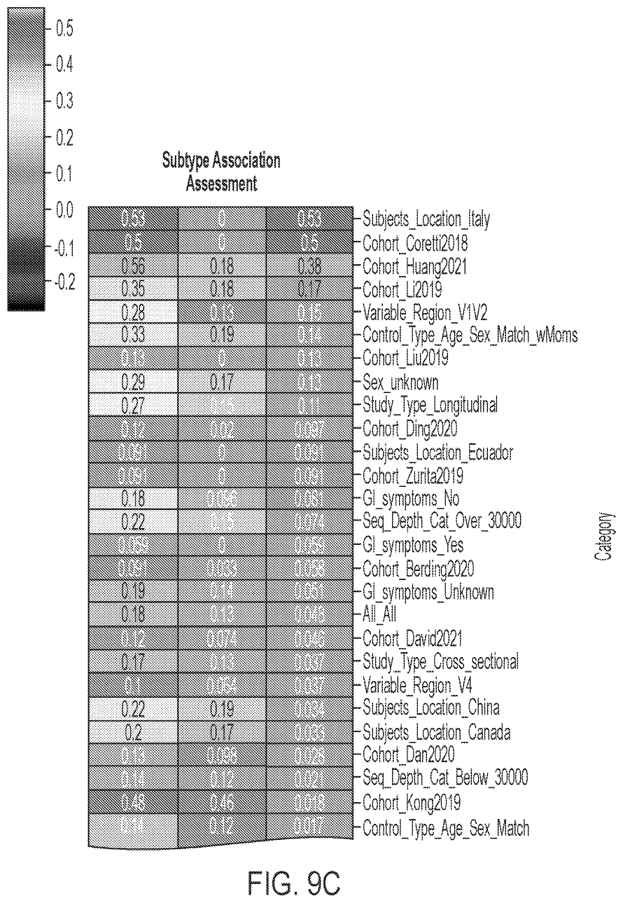

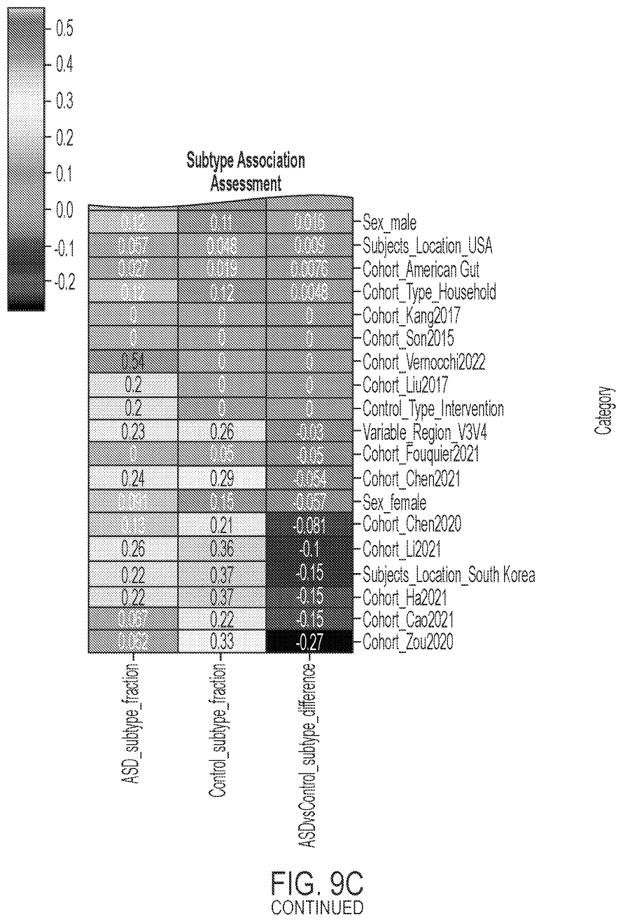

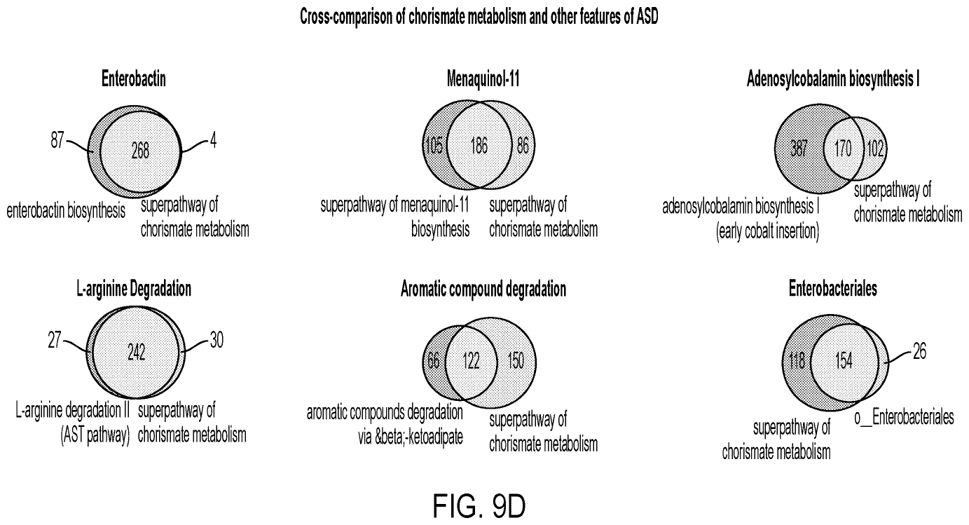

FIG. 9A is a bubble plot showing the most commonly significant pathways found in cross-sectional cohorts of the ASD gut microbiome and controls. Each point is colored by the Log 2 compositional fold-change between ASD and control subjects and sized by whether the association passed a significance threshold of a P<0.05 via ranksum testing without adjusting for multiple-hypothesis testing. Pathways related to chorismate are indicated by arrows. These pathways include: superpathway of menaquinol-7 biosynthesis, superpathway of chorismate metabolism, aromatic compounds degradation via beta-ketoadipate, superpathway of menaquinol-12 biosynthesis, superpathway of menaquinol-11 biosynthesis, superpathway of demethylmenaquinol-8 biosynthesis, superpathway of menaquinol-8 biosynthesis, superpathway of menaquinol-13 biosynthesis, and enterobactin biosynthesis.

FIG. 9B shows histograms of compositional abundance (when present) of the superpathway of chorismate metabolism in the gut microbiome for ASD and control subjects. FIG. 9B also shows overlaid KDE curves for ASD and control subjects. An optimal discriminatory boundary for classifying ASD vs. controls was determined and plotted in FIG. 9B.

FIG. 9C is a heat map describing the fractional abundance of ASD or control subjects containing more than a threshold abundance of superpathway of chorismate metabolism in their gut microbiome, with the data set divided by cohorts and study factors. In addition, the difference between the ASD fractional abundance and the control fractional abundance was plotted, showing the cohorts and study factors where this feature was highly enriched in ASD subjects.

FIG. 9D shows a cross-comparison of chorismate metabolism and other features of the microbiome. Discriminatory thresholds were identified for various features linked to ASD status, and ASD subjects identified as having high levels of each category were compared to the ASD subjects identified as having increased levels of chorismate metabolism. FIG. 9D shows Venn diagrams describing the overlap between subjects categorized as having increased abundance of each feature listed. As shown in FIG. 9D, a strong overlap was identified in patients with increased enterobactin genes, menaquinol-11 genes, aromatic compound degradation, and L-arginine degradation. The chorismate metabolism phenotype may often be driven by enterobacteriales as those with increased enterobacteriales were almost entirely represented by those with increased chorismate metabolism. Accordingly, in some embodiments, compositional abundance of enterobacteriales, L-arginine degradation, and/or enterobactin may be used to assess whether a patient is likely to respond to chorismate interventions.

### Metabolic Flux Differences

Metabolic flux differences between ASD and control subjects were analyzed using the computational tool MICOM. MICOM uses metabolic models built around the known nutrients required and metabolic pathways within microorganisms and simulates how the collective species identified in a microbiome would interact with each other given the diet being taken in. For these studies, a standard “western diet” was utilized. A customized “western diet” that increased the amount of chorismate available from the diet was also developed.

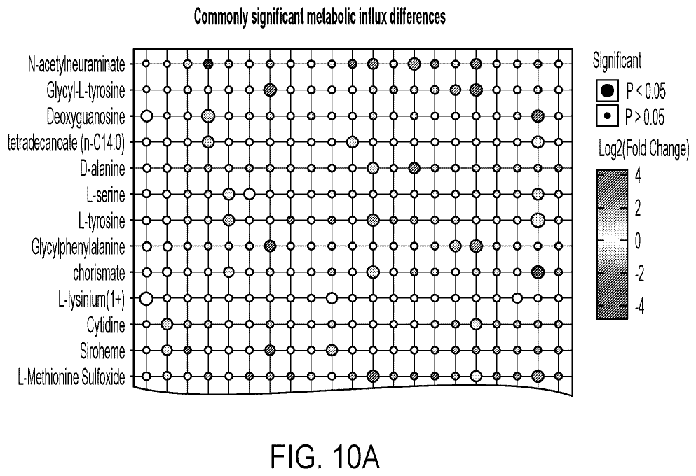

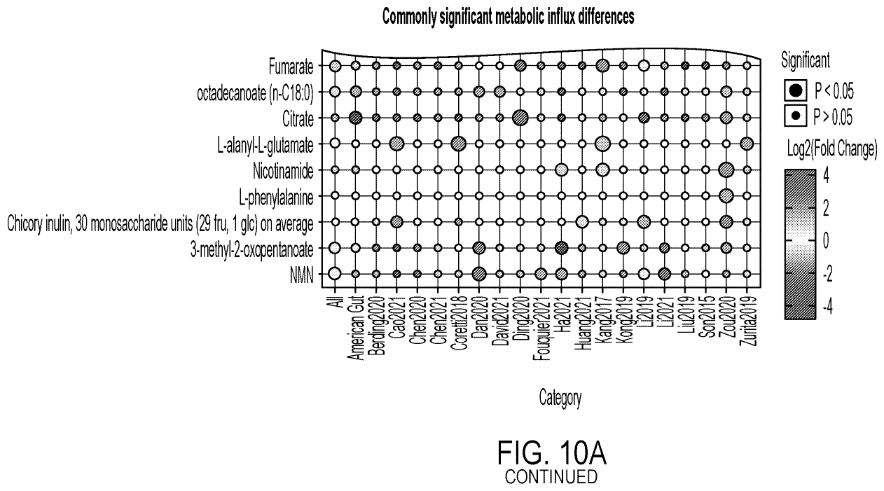

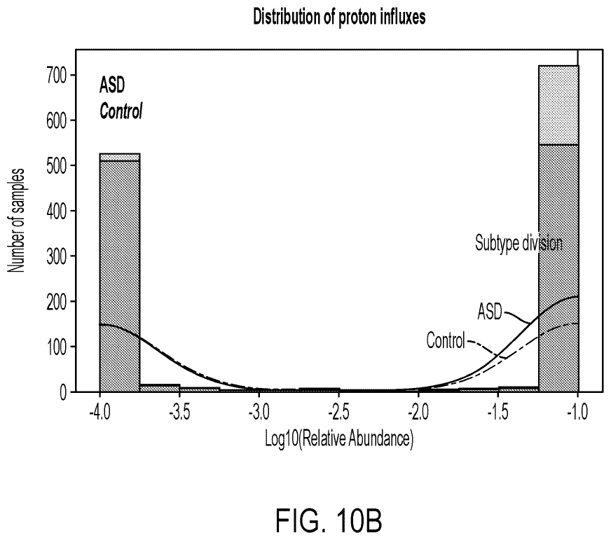

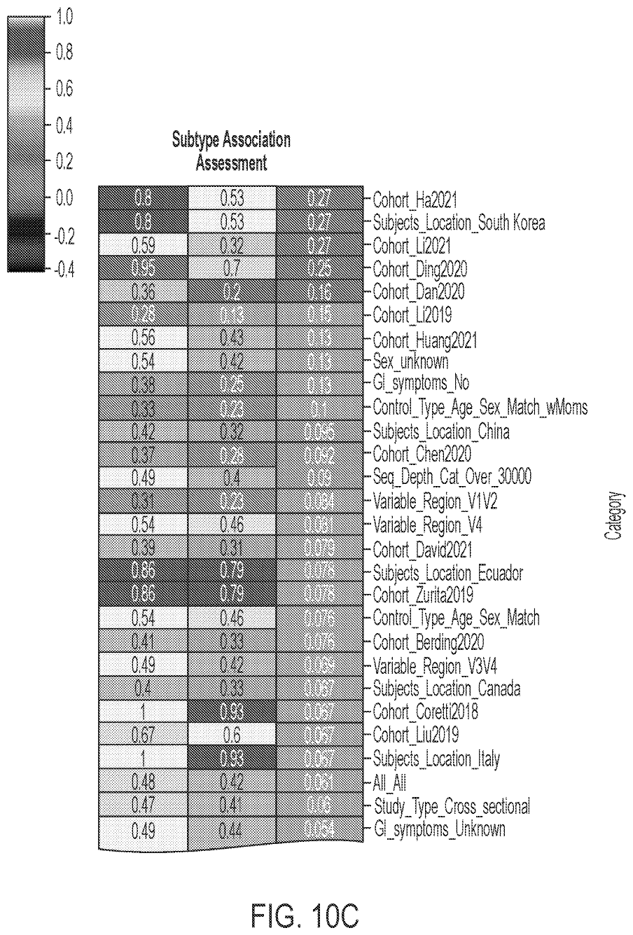

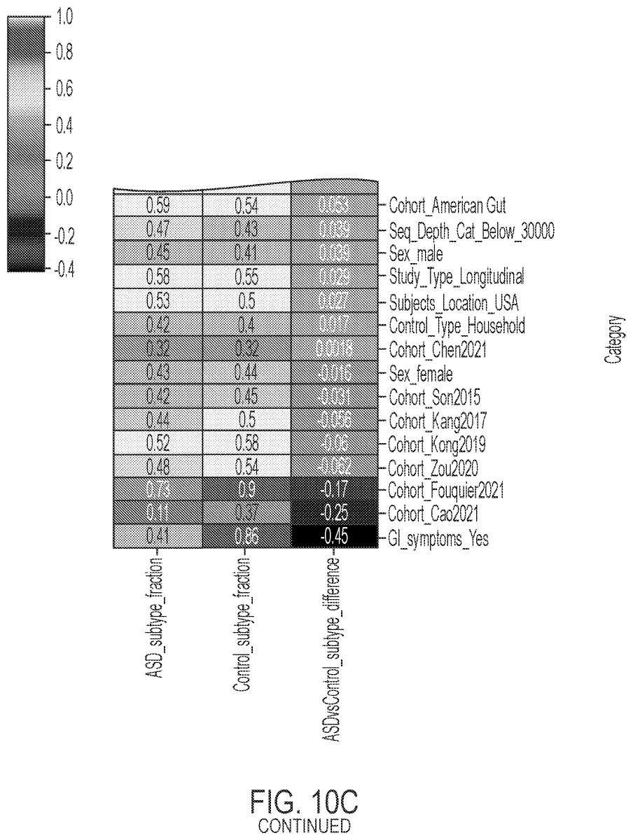

FIG. 10A is a bubble plot showing the most commonly significant pathways found in cross-sectional cohorts of the ASD gut microbiome and controls. Each point is colored by the Log 2 fold-change between ASD and control subjects and sized by whether the association passed a significance threshold of a P<0.05 via ranksum testing without adjusting for multiple-hypothesis testing. Among the most commonly different metabolic flux differences between ASD and control subjects were nicotinamide mononucleotide (NMN), 3-methyl-2-oxopentanoate, L-phenylalanine, nicotinamide, fumarate, chorismate, L-tyrosine, L-serine, glycyl-L-tyrosine, and glycylphenylalanine.

Upon examining different features for their potential to correspond to subgroups of ASD subjects, proton flux appeared to be a promising candidate where 14% of ASD subjects' microbiome indicated a high influx of free protons. This phenotype was particularly pronounced in the Ding2020 and Kong2019 cohorts as well as among the studies conducted in Ecuador, South Korea, and using the V3V4 variable region to profile the gut microbiome. FIG. 10B shows histograms with overlaid KDE curves describing the distribution of the influx (when present) of protons by the gut microbiome of ASD and control subjects. An optimal discriminatory boundary for classifying ASD vs. controls was determined and plotted in FIG. 10B.

FIG. 10C is a heat map describing the fractional abundance of ASD or control subjects containing more than a threshold influx of protons by their gut microbiome, with the data set divided by cohorts and study factors. In addition, the difference between the ASD fractional abundance and the control fractional abundance is plotted, showing the cohorts and study factors where this feature was highly enriched in ASD subjects.

The metabolic influx analyses further support claims based on the functional analyses of the gut microbiome of ASD (modulating chorismate-pathway molecules for ASD treatment), as chorismate-related metabolites are frequently identified as molecules with differential intake by the gut microbiota of ASD subjects. These molecules include L-Phenylalanine, L-Tyrosine, and chorismate. The results further suggest that NMN, fumarate, and/or protons may be important to ASD pathology. For example, the increased proton flux found in some ASD subjects may indicate their intestinal pH is more acidic. A potential intervention for these subjects could be decreasing the GI pH. Accordingly, in some embodiments, a therapeutic intervention comprises administering one or more compositions to decrease the GI pH of a subject having or at risk of ASD.

### Cross-Comparison of Major Subtypes

Having identified several promising biomarkers of ASD, subjects within each group were cross compared to identify if these were distinct subtypes of ASD. For this analysis, ASD subjects beyond the set threshold for the super-pathway of chorismate metabolism were compared to ASD subjects containing the biomarker “Sarcina” and to patients containing high levels of proton influx. The results found very little overlap in these ASD populations, suggesting that they may be distinct subtypes of ASD that may necessitate a diagnostic approach for identifying each subtype to identify which targeted therapeutic may work best for the individual. For the largest subtype, it was estimated that at least 18% of ASD subjects may benefit from an intervention based on chorismate. It was further estimated that at least 13.8% of ASD subjects may benefit from an intervention based on GI pH adjustment and 13.5% of ASD subjects may benefit from interventions targeting Clostridium ventriculi.

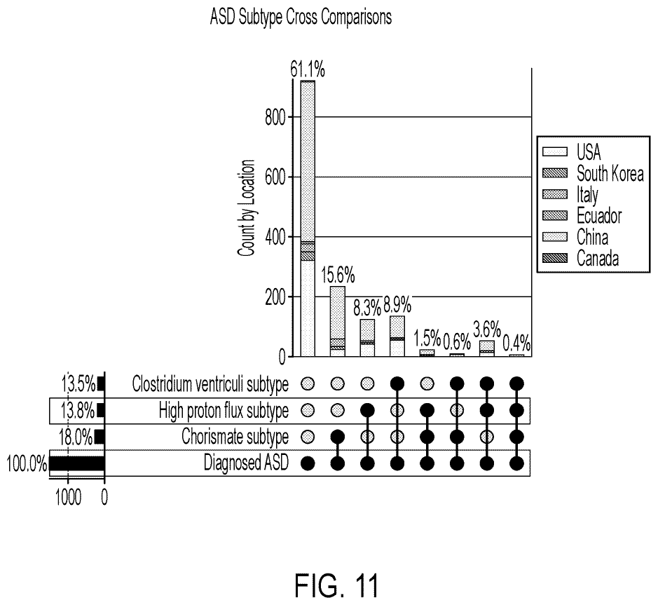

FIG. 11 shows an upset plot cross-comparing the patient populations of the three leading subtypes of ASD from this Example. On the bottom left, a bar plot shows the number of patients in each subtype with corresponding percentages. On the bottom right are indications of the participation in a particular subtype corresponding to each bar plot above. Each bar plot shows the number of ASD subjects with participation within each subtype, and each bar is colored depending on the country of the ASD subject. Percentages of the total population represented by each subpopulation are plotted above each bar.

### Chorismate Intervention

Having identified a subpopulation of ASD which had a higher capacity for metabolizing chorismate and the molecules derived from chorismate, the impact that increasing chorismate in the diet of these patients would have on the metabolic intake of patients' microbiomes was evaluated. For this analysis, the dietary flux of chorismate was increased by an order of magnitude, and MICOM simulations were re-run on all subjects in the meta-analysis study. The total influx of each metabolite by the gut microbiota was compared to the influx obtained from the standard diet. The difference in influx among the chorismate subtype of ASD was compared to all other ASD subjects, and the highest statistically significant metabolites between these groups were ranked. The analysis identified strikingly significant differences in several metabolites that may be key in the pathology of ASD, including indole, a derivative of tryptophan with behavioral impacts, folate, chorismate, L-tyrosine, and menaquinone 8. These studies also identified differences in other interesting metabolites, including 3-methyl-2-oxopentanoate, which is a known neurotoxin, and NMN. The mean influx of each of these metabolites was decreased by chorismate intervention in these subjects while not changing significantly in the greater population. This decreased influx of these metabolites by the gut microbiota may indicate that the increased availability of chorismate precludes the necessity of certain species to degrade the metabolites downstream of chorismate. This further indicates that a chorismate intervention on this patient population may have the intended mechanism of action by decreasing the amount of degradation of various metabolites downstream of chorismate in compensation for the decreased availability of chorismate due to glyphosate exposure or other mechanisms.

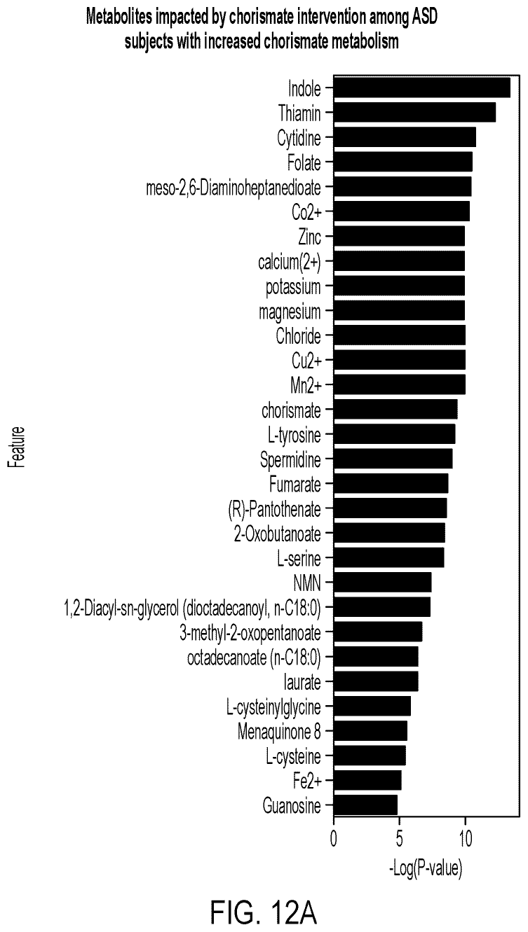

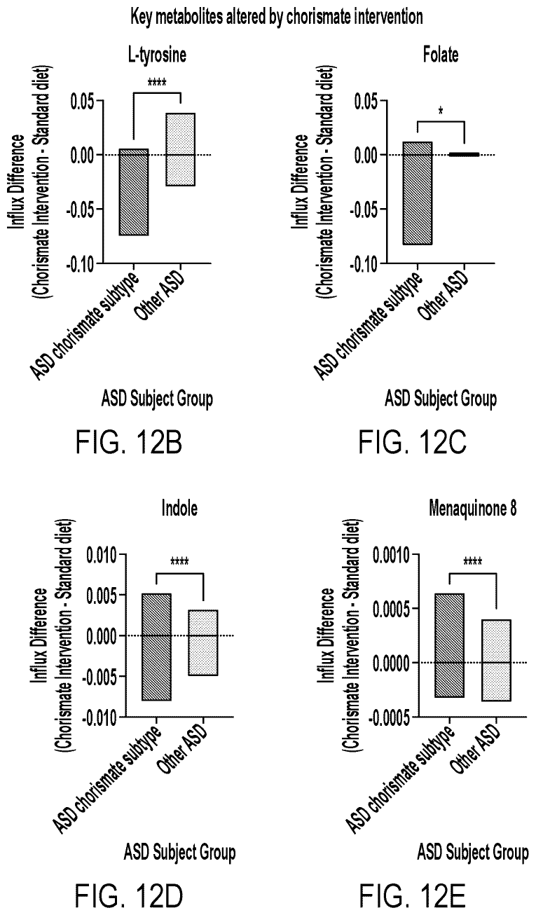

FIG. 12A shows the top 30 metabolites altered among ASD subjects with increased chorismate metabolism genes when given increased chorismate. A barplot is depicted of the −Log 10 (FDR-adjusted p-values) based on ranksum statistical comparison of the means between flux differences on and off of chorismate modified western diet for chorismate and non-chorismate ASD subtypes.

FIGS. 12B-12E show boxplots showing the distribution of flux differences for ASD of the chorismate subtype or other subjects when given increased chorismate in their simulated diet. Above each boxplot are significance markers based on unpaired Welch-corrected T tests.

The simulated chorismate intervention studies indicate that chorismate may be utilized to alter levels of key metabolites (e.g., L-tyrosine, folate, indole, menaquinone 8) in certain ASD subjects. They demonstrate that administration of chorismate may be likely to decrease reliance of gut microbes on intaking metabolites downstream of chorismate. Given the prior findings regarding these downstream metabolites on various symptoms or comorbidities of ASD, it suggests that chorismate may intervene through these mechanisms.

### Example 3

In this Example, chorismate is administered to treat two distinct rodent models of ASD: prenatal exposure to either glyphosate (also referred to as Roundup) or valproic acid.

Autism spectrum disorder emerges from distinct etiologies: altered microbiome, viral infection, and genetics. A subset of ASD patients may possess alterations along the chorismate metabolic pathway encoded by their gut microbiota. Chorismate supplementation may ameliorate ASD symptoms and may be a possible treatment for at least a “chorismate subtype” of ASD. In this Example, chorismate is administered to treat two distinct rodent models of ASD: prenatal exposure to either glyphosate (also referred to as Roundup) or valproic acid. Glyphosate directly interferes with the synthesis of chorismate by the Shikimate pathway and has been shown to induce ASD-like behaviors in rodents. The exact mechanism of action of valproic acid remains unclear, but rodents born to mothers exposed to valproic acid also exhibit many ASD-like behavioral phenotypes.

This Example focuses on the cardinal behavioral symptom of rodent models of ASD: lack of sociability. This is measured using the 3-chamber assay described below. Secondary measures include the metabolic and microbial profile of the gut microbiome.

### Experimental Methods

### Induction—Valproic Acid

Timed pregnant C57Bl/6 females are administered a vehicle (0.9% saline) or valproic acid (VPA) (500 mg/kg; 500 μL; intraperitoneal) on Gestational Day 12.5. Male pups are weaned at 3 weeks of age (P21); female pups are euthanized.

### Induction—Glyphosate

Timed pregnant C57Bl/6 females are administered regular drinking water or Glyphosate-infused water (175 mg/kg; about 0.1%) from Embryonic Day 5 (E5) until Postnatal Day 21 (P21) for a total of 37 days. Male pups are weaned at 3 weeks of age (P21); female pups are euthanized.

### Treatment—Chorismate

Upon weaning (P21), animals are group housed, weighed and monitored daily, and fecal samples are collected weekly. Half of the animals in each group are administered either regular drinking water or Chorismic acid in the drinking water. Drinking water is changed every 24 hours.

### Readouts—Sociability

The primary behavioral readout is animal sociability using the 3-chamber assay. In this assay, a rectangular arena has 3 chambers, and the subject mouse is allowed to freely walk between the chambers.

On P56, the mouse is allowed to habituate for 5 minutes in the chamber. Then, a pair of empty, small wire cages is placed on either side of the left and right chambers. In one of the wire cages, an unfamiliar (stranger) mouse is introduced. The subject mouse is allowed 10 minutes to freely explore between the three chambers. Video tracking records the amount of time the subject mouse spends in each chamber.

On P66, animals are euthanized. Blood (plasma), colon contents (flash-frozen), cecum contents (flash-frozen), and brain (flash-frozen) are collected at euthanasia.

### Expected Results

Previous studies have shown that control subject mice (Controls for Induction and Treatment) prefer to spend 2-5× more time in the chamber with the stranger mouse than the chamber with the empty cage.

These studies have also shown that subject mice with prenatal exposure to valproic acid or glyphosate spend roughly equal times between chambers-exhibiting no social preference: a key ASD-like behavioral phenotype.

In contrast, subject mice with prenatal exposure to valproic acid or glyphosate but treated with chorismic acid should exhibit social preference similar to that of control mice: spending 2-5× more time in the chamber with the stranger mouse than the chamber with the empty cage.

It is also hypothesized that chorismate is necessary for normal neurodevelopment, but is not a substrate for promoting sociability. That is, control mice (Controls for Induction) treated with chorismic acid do not exhibit a heighted sociability when compared to control mice (Controls for Induction and Treatment).

TABLE 1
Genes involved in the superpathway of chorismate metabolism.

| | Gene | Gene | | | |
|---|---|---|---|---|---|
| Gene ID | Accession | name | | Reaction EC | Enzymatic activity |
| EG10508 | b0421 | ispA | GPPSYN-RXN | 2.5.1.1 | geranyl diphosphate synthase |
| EG10079 | b0754 | aroG | DAHPSYN-RXN | 2.5.1.54 | 3-deoxy-7-phosphoheptulonate |
| | | | | | synthase |
| EG10080 | b1704 | aroH | DAHPSYN-RXN | 2.5.1.54 | 3-deoxy-7-phosphoheptulonate |
| | | | | | synthase |
| EG10078 | b2601 | aroF | DAHPSYN-RXN | 2.5.1.54 | 3-deoxy-7-phosphoheptulonate |
| | | | | | synthase |
| EG10262 | b0583 | entD | ENTDB-RXN | 2.7.8.7 | phosphopantetheinyl |
| | | | | | transferase |
| EG12221 | b3475 | acpT | ENTDB-RXN | 2.7.8.7 | holo-[acyl carrier protein] |
| | | | | | synthase |
| EG10247 | b2563 | acpS | ENTDB-RXN | 2.7.8.7 | holo-[acyl-carrier-protein] |
| | | | | | synthase |
| EG10264 | b0586 | entF | ENTF-RXN | No EC# | L-serine--[holo EntF] ligase |
| EG10260 | b0595 | entB | ENTF-RXN | No EC# | enterobactin synthase |
| EG10262 | b0583 | entD | ENTF-RXN | No EC# | enterobactin synthase |
| EG10264 | b0586 | entF | ENTF-RXN | No EC# | enterobactin synthase |
| EG10263 | b0594 | entE | ENTF-RXN | No EC# | enterobactin synthase |
| EG10262 | b0583 | entD | RXN-15889 | 2.7.8.7 | phosphopantetheinyl |
| | | | | | transferase |
| EG11375 | b2153 | folE | GTP-CYCLOHYDRO-I-RXN | 3.5.4.16 | GTP cyclohydrolase I |
| EG11138 | b1865 | nudB | H2NEOPTERINP3PYROPHOSPHOHYDRO-RX | 3.6.1.67 | dihydroneopterin triphosphate |
| | | | | | diphosphatase |
| EG11673 | b3058 | folB | H2NEOPTERINALDOL-RXN | 4.1.2.25 | dihydroneopterin aldolase |
| EG11374 | b0142 | folK | H2PTERIDINEPYROPHOSPHOKIN-RXN | 2.7.6.3 | 2-amino-4-hydroxy-6- |
| | | | | | hydroxymethyldihydropteridine |
| | | | | | diphosphokinase |
| EG10264 | b0586 | entF | RXN-15890 | No EC# | L-serine--[holo EntF] ligase |
| EG10260 | b0595 | entB | RXN-15890 | No EC# | enterobactin synthase |
| EG10262 | b0583 | entD | RXN-15890 | No EC# | enterobactin synthase |
| EG10264 | b0586 | entF | RXN-15890 | No EC# | enterobactin synthase |
| EG10263 | b0594 | entE | RXN-15890 | No EC# | enterobactin synthase |
| EG10074 | b3389 | aroB | 3-DEHYDROQUINATE-SYNTHASE-RXN | 4.2.3.4 | 3-dehydroquinate synthase |
| EG10076 | b1693 | aroD | 3-DEHYDROQUINATE-DEHYDRATASE- | 4.2.1.10 | 3-dehydroquinate dehydratase |
| | | | RXN | | |
| EG10077 | b3281 | aroE | SHIKIMATE-5-DEHYDROGENASE-RXN | 1.1.1.25 | shikimate dehydrogenase |
| EG10082 | b0388 | aroL | SHIKIMATE-KINASE-RXN | 2.7.1.71 | shikimate kinase |
| EG10081 | b3390 | aroK | SHIKIMATE-KINASE-RXN | 2.7.1.71 | shikimate kinase |
| EG10073 | b0908 | aroA | 2.5.1.19-RXN | 2.5.1.19 | 3-phosphoshikimate 1- |
| | | | | | carboxyvinyltransferase |
| EG10075 | b2329 | aroC | CHORISMATE-SYNTHASE-RXN | 4.2.3.5 | chorismate synthase |
| EG11028 | b1264 | trpE | ANTHRANSYN-RXN | 4.1.3.27 | anthranilate synthase |
| EG11027 | b1263 | trpD | ANTHRANSYN-RXN | 4.1.3.27 | anthranilate synthase |
| EG11027 | b1263 | trpD | PRTRANS-RXN | 2.4.2.18 | anthranilate phosphoribosyl |
| | | | | | transferase |
| EG11026 | b1262 | trpC | PRAISOM-RXN | 5.3.1.24 | phosphoribosylanthranilate |
| | | | | | isomerase |
| EG11026 | b1262 | trpC | IGPSYN-RXN | 4.1.1.48 | indole-3-glycerol phosphate |
| | | | | | synthase |
| EG11024 | b1260 | trpA | RXN0-2381 | 4.1.2.8 | indoleglycerol phosphate |
| | | | | | aldolase |
| EG11024 | b1260 | trpA | RXN0-2381 | 4.1.2.8 | tryptophan synthase |
| EG11025 | b1261 | trpB | RXN0-2381 | 4.1.2.8 | tryptophan synthase |
| EG11025 | b1261 | trpB | RXN0-2382 | 4.2.1.122 | tryptophan synthase |
| EG11024 | b1260 | trpA | RXN0-2382 | 4.2.1.122 | tryptophan synthase |
| EG11025 | b1261 | trpB | RXN0-2382 | 4.2.1.122 | tryptophan synthase |
| EG10707 | b2599 | pheA | CHORISMATEMUT-RXN | 5.4.99.5 | chorismate mutase |
| EG11039 | b2600 | tyrA | CHORISMATEMUT-RXN | 5.4.99.5 | chorismate mutase |
| EG10707 | b2599 | pheA | PREPHENATEDEHYDRAT-RXN | 4.2.1.51 | prephenate dehydratase |
| EG10497 | b3770 | ilvE | RXN-10814 | 2.6.1.1/2.6.1.27/2.6.1.57 | phenylalanine transaminase |
| EG10096 | b0928 | aspC | RXN-10814 | 2.6.1.1/2.6.1.27/2.6.1.57 | phenylalanine transaminase |
| EG11040 | b4054 | tyrB | RXN-10814 | 2.6.1.1/2.6.1.27/2.6.1.57 | phenylalanine aminotransferase |
| EG11039 | b2600 | tyrA | PREPHENATEDEHYDROG-RXN | 1.3.1.12 | prephenate dehydrogenase |
| EG10096 | b0928 | aspC | TYROSINE-AMINOTRANSFERASE-RXN | 2.6.1.1/2.6.1.5/2.6.1.27/2.6.1.57 | tyrosine aminotransferase |
| EG11040 | b4054 | tyrB | TYROSINE-AMINOTRANSFERASE-RXN | 2.6.1.1/2.6.1.5/2.6.1.27/2.6.1.57 | tyrosine aminotransferase |
| EG10261 | b0593 | entC | ISOCHORSYN-RXN | 5.4.4.2 | isochorismate synthase |
| EG12362 | b2265 | menF | ISOCHORSYN-RXN | 5.4.4.2 | isochorismate synthase |
| EG10579 | b2264 | menD | 2.5.1.64-RXN | 2.2.1.9 | 2-succinyl-5-enolpyruvyl-6- |
| | | | | | hydroxy-3-cyclohexene-1- |
| | | | | | carboxylate synthase |
| EG12438 | b2263 | menH | RXN-9310 | 4.2.99.20 | (1R,6R)-2-succinyl-6-hydroxy- |
| | | | | | 2,4-cyclohexadiene-1- |
| | | | | | carboxylate synthase |
| EG11532 | b2261 | menC | O-SUCCINYLBENZOATE-COA-SYN-RXN | 4.2.1.113 | o-succinylbenzoate synthase |
| EG12437 | b2260 | menE | O-SUCCINYLBENZOATE-COA-LIG-RXN | 6.2.1.26 | o-succinylbenzoate--CoA |
| | | | | | ligase |
| EG11368 | b2262 | menB | NAPHTHOATE-SYN-RXN | 4.1.3.36 | 1,4-dihydroxy-2-naphthoyl- |
| | | | | | CoA synthase |
| G6912 | b1686 | menI | RXN-9311 | 3.1.2.28 | 1,4-dihydroxy-2-naphthoyl- |
| | | | | | CoA hydrolase |
| EG11880 | b3930 | menA | DMK-RXN | 2.5.1.74 | 1,4-dihydroxy-2-naphthoate |
| | | | | | octaprenyltransferase |
| EG11473 | b3833 | ubiE | ADOMET-DMK-METHYLTRANSFER-RXN | 2.1.1.163 | demethylmenaquinone |
| | | | | | methyltransferase |
| EG10260 | b0595 | entB | ISOCHORMAT-RXN | 3.3.2.1 | isochorismatase |
| EG10259 | b0596 | entA | DHBDEHYD-RXN | 1.3.1.28 | 2,3-dihydro-2,3- |
| | | | | | dihydroxybenzoate |
| | | | | | dehydrogenase |
| EG10263 | b0594 | entE | DHBAMPLIG-RXN | No EC# | 2,3-dihydroxybenzoate-[aryl- |
| | | | | | carrier protein] ligase |
| EG10263 | b0594 | entE | RXN0-6442 | No EC# | 2,3-dihydroxybenzoate-[aryl- |
| | | | | | carrier protein] ligase |
| EG10264 | b0586 | entF | RXN-15891 | No EC# | 2,3-dihydroxybenzoyl- |
| | | | | | [EntB]:seryl-[EntF] 2,3- |
| | | | | | dihydroxybenzoate transferase |
| EG10260 | b0595 | entB | RXN-15891 | No EC# | enterobactin synthase |
| EG10262 | b0583 | entD | RXN-15891 | No EC# | enterobactin synthase |
| EG10264 | b0586 | entF | RXN-15891 | No EC# | enterobactin synthase |
| EG10263 | b0594 | entE | RXN-15891 | No EC# | enterobactin synthase |
| EG10264 | b0586 | entF | ENTG-RXN | No EC# | enterobactin synthase |
| EG10260 | b0595 | entB | ENTG-RXN | No EC# | enterobactin synthase |
| EG10262 | b0583 | entD | ENTG-RXN | No EC# | enterobactin synthase |
| EG10264 | b0586 | entF | ENTG-RXN | No EC# | enterobactin synthase |
| EG10263 | b0594 | entE | ENTG-RXN | No EC# | enterobactin synthase |
| EG10683 | b1812 | pabB | PABASYN-RXN | 2.6.1.85 | 4-amino-4-deoxychorismate |
| | | | | | synthase |
| EG10682 | b3360 | pabA | PABASYN-RXN | 2.6.1.85 | 4-amino-4-deoxychorismate |
| | | | | | synthase |
| EG11493 | b1096 | pabC | ADCLY-RXN | 4.1.3.38 | aminodeoxychorismate lyase |
| EG50011 | b3177 | folP | H2PTEROATESYNTH-RXN | 2.5.1.15 | dihydropteroate synthase |
| EG10327 | b2315 | folC | DIHYDROFOLATESYNTH-RXN | 6.3.2.12 | dihydrofolate synthetase |
| EG10326 | b0048 | folA | DIHYDROFOLATEREDUCT-RXN | 1.5.1.3 | dihydrofolate reductase |
| G6862 | b1606 | folM | DIHYDROFOLATEREDUCT-RXN | 1.5.1.3 | dihydrofolate reductase |
| EG10508 | b0421 | ispA | FPPSYN-RXN | 2.5.1.10 | farnesyl diphosphate synthase |
| EG10017 | b3187 | ispB | RXN-8992 | 2.5.1.90 | all-trans-octaprenyl- |
| | | | | | diphosphate synthase |
| EG11369 | b4039 | ubiC | CHORPYRLY-RXN | 4.1.3.40 | chorismate lyase |
| EG11370 | b4040 | ubiA | 4OHBENZOATE-OCTAPRENYLTRANSFER- | 2.5.1.39 | 4-hydroxybenzoate |
| | | | RXN | | octaprenyltransferase |
| EG11396 | b3843 | ubiD | 3-OCTAPRENYL-4-OHBENZOATE- | 4.1.1.98 | 3-octaprenyl-4- |
| | | | DECARBOX-RXN | | hydroxybenzoate |
| | | | | | decarboxylase |
| EG11333 | b2906 | ubiI | 2-OCTAPRENYLPHENOL-HYDROX-RXN | 1.14.13.240 | 2-octaprenylphenol 6- |
| | | | | | hydroxylase |
| EG11143 | b2232 | ubiG | 2-OCTAPRENYL-6-OHPHENOL-METHY- | 2.1.1.222 | 2-octaprenyl-6-hydroxyphenol |
| | | | RXN | | methylase |
| EG11324 | b2907 | ubiH | 2-OCTAPRENYL-6-METHOXYPHENOL- | 1.14.13.M56 | 2-octaprenyl-6-methoxyphenol |
| | | | HYDROX-RXN | | 4-hydroxylase |
| EG11473 | b3833 | ubiE | 2-OCTAPRENYL-METHOXY-BENZOQ- | 2.1.1.201 | 2-octaprenyl-6-methoxy-1,4- |
| | | | METH-RXN | | benzoquinol methylase |
| G6365 | b0662 | ubiF | OCTAPRENYL-METHYL-METHOXY- | 1.14.99.60 | 2-octaprenyl-3-methyl-6- |
| | | | BENZOQ-OH-RXN | | methoxy-1,4-benzoquinol |
| | | | | | hydroxylase |
| EG11143 | b2232 | ubiG | DHHB-METHYLTRANSFER-RXN | 2.1.1.64 | 3-demethylubiquinone-8 3-O- |
| | | | | | methyltransferase |

TABLE 2
Metabolites involved in the superpathway of chorismate metabolism.

| Molecule | Synonyms | Subpathway |
|---|---|---|
| D-erythrose 4- | erythrose-4P; threose 4-phosphate; erythrose-4-phosphate; | Shikimate |
| phosphate | erythrose-4-P; D-erythrose-4-P | Pathway |
| 3-deoxy-D-arabino- | 3-deoxy-D-arabino-heptulosonate-7-P, 3-deoxy-arabino- | Shikimate |
| heptulosonate 7- | heptulosonate 7-phosphate, 3-deoxy-arabino-heptulosonate-7- | Pathway |
| phosphate | P, 2-dehydro-3-deoxy-D-arabino-heptonate 7-phosphate, 3-deoxy- | |
| | D-arabino-hept-2-ulosonate 7-phosphate, 3-deoxy-arabino- | |
| | heptulonate 7-phosphate, DAHP | |
| 3-dehydroquinate | DEHYDROQUINATE; 3-dehydroquinic acid; 5-dehydroquinic | Shikimate |
| | acid; 5-dehydroquinate; 5-de-H-quinate; rel-(1R,3R,4S)-1,3,4- | Pathway |
| | trihydroxy-5-oxocyclohexanecarboxylate | |
| 3-dehydroshikimate | 3-dehydroshikimic acid; 5-dehydroshikimic acid; 5- | Shikimate |
| | dehydroshikimate | Pathway |
| shikimate | shikimic acid; (3R,4S,5R)--3,4,5-trihydroxycyclohex-1-ene-1- | Shikimate |
| | carboxylate | Pathway |
| shikimate 3-phosphate | shikimate 5-phosphate; shikimate-5-P; 3- | Shikimate |
| | phosphoshikimate; shikimate-3-P | Pathway |
| 5-enolpyruvoyl- | 3-enolpyruvyl-shikimate 5-phosphate; 3-enolpyruvyl-shikimate- | Shikimate |
| shikimate 3-phosphate | 5-P; 5-O-(1-carboxyvinyl)-3-phosphoshikimate; 5-enolpyruvyl- | Pathway |
| | shikimate 3-phosphate | |
| chorismate | chorismic acid | Shikimate |
| | | Pathway |
| anthranilate | anthranilic acid; 2-aminobenzoic acid; vitamin L1; o- | L-Tryptophan |
| | aminobenzoic acid; 2-aminobenzoate; o-aminobenzoate | Biosynthesis |
| N-(5-phosphoribosyl)- | N-(5-phospho-D-ribosyl)-anthranilate; N-(5-phospho-β-D- | L-Tryptophan |
| anthranilate | ribosyl)-anthranilate; 5-phosphoribosyl-anthranilate; 5-P-ribosyl- | Biosynthesis |
| | anthranilate; 5′-phosphoribosyl-anthranilate; 5′-P-ribosyl- | |
| | anthranilate; N-(5-phosphoribosyl)-anthranilate | |
| 1-(2- | 1-(o-carboxyphenylamino)-1-deoxyribulose-5′-P; 1-(2- | L-Tryptophan |
| carboxyphenylamino)- | carboxyphenylamino)-1-deoxy-D-ribulose-5-P; 1-(o- | Biosynthesis |
| 1-deoxy-D-ribulose 5- | carboxyphenylamino)-1′-deoxyribulose 5′-phosphate | |
| phosphate | | |
| (1S,2R)-1-C-(indol-3- | C1-(3-Indolyl)-glycerol 3-phosphate; indole-3-glycerol-P; 1- | L-Tryptophan |
| yl)glycerol 3- | (indol-3-yl)glycerol-3-P; 1-(indol-3-yl)glycerol-3- | Biosynthesis |
| phosphate | phosphate; indoleglycerol phosphate; indole-3-glycerol-phosphate | |
| Indole | | L-Tryptophan |
| | | Biosynthesis |
| L-serine | S; serine; L-ser | L-Tryptophan |
| | | Biosynthesis |
| L-tryptophan | W; tryptacin; trofan; tryptophan; 2-amino-3-indolylpropanic | L-Tryptophan |
| | acid; L-trp; trp | Biosynthesis |
| L-glutamine | Q; proglumide; glum; glutamine; glumin; 2-aminoglutaramic | L-Tryptophan |
| | acid; glutamic acid 5-amide; L-gln | Biosynthesis |
| 5-phospho-α-D-ribose | 5-phosphoribosyl 1-pyrophosphate; 5-phosphoribosyl | L-Tryptophan |
| 1-diphosphate | diphosphate; 5-phosphoribosyl-1-PP; 5-phosphoribosyl-PP; 5- | Biosynthesis |
| | phosphoribosyl-1-pyrophosphate; 5- | |
| | phosphoribosylpyrophosphate; phosphoribosylpyrophosphate; 5- | |
| | phospho-ribosyl-pyrophosphate; α-D-5-phosphoribosylPP; α-D-5- | |
| | P-RibosylPP | |

What is claimed is:

1. A method of assisting in the diagnosis of a subject having one or more symptoms of autism, the method comprising determining a level of chorismate and/or a molecule within a chorismate metabolic pathway in a sample obtained from the subject.

2. The method of claim 1, wherein the molecule within the chorismate metabolic pathway is an upstream or downstream metabolite of chorismate.

3. The method of claim 2, wherein the downstream metabolite of chorismate is tryptophan, tyrosine, phenylalanine, or a metabolite thereof.

4. The method of claim 3, wherein the downstream metabolite of chorismate is a downstream metabolite of tryptophan, wherein the downstream metabolite of tryptophan is serotonin, melatonin, kyneurenine, and/or an indole.

5. The method of claim 2, wherein the downstream metabolite of chorismate is folate or a derivative thereof.

6. The method of claim 2, wherein the downstream metabolite of chorismate is a siderophore.

7. The method of claim 6, wherein the siderophore is enterobactin.

8. The method of claim 2, wherein the downstream metabolite of chorismate is a ubiquinol or a menaquinol.

9. The method of any one of claims 1-8, wherein the sample is a fecal sample.

10. The method of any one of claims 1-8, wherein the sample is a gastrointestinal sample.

11. The method of claim 10, wherein the gastrointestinal sample is a gastric aspirate, biopsy, intestinal fluid, endoscopic brush, and/or laser capture microdissection sample.

12. The method of any one of claims 1-8, wherein the sample is a salivary, buccal, nasal, urine, serum, or cerebrospinal fluid sample.

13. The method of any one of claims 1-12, wherein determining the level of chorismate and/or the molecule within the chorismate metabolic pathway comprises amplifying and/or sequencing one or more portions of one or more nucleic acids present in the sample.

14. The method of claim 13, wherein the one or more portions of one or more nucleic acids comprise one or more regions of 16S ribosomal RNA present in the sample.

15. The method of claim 13, wherein amplifying and/or sequencing one or more portions of the one or more nucleic acids of the sample comprises conducting quantitative PCR on at least a portion of the sample.

16. The method of claim 13, wherein amplifying and/or sequencing one or more portions of the one or more nucleic acids of the sample comprises conducting shotgun metagenomic sequencing on at least a portion of the sample.

17. The method of any one of claims 1-16, wherein determining the level of chorismate and/or the molecule within the chorismate metabolic pathway comprises using HPLC, LC-MS, GC-MS, and/or MALDI-TOF.

18. The method of any one of claims 1-17, further comprising determining, if the level of chorismate and/or the molecule within the chorismate metabolic pathway is higher or lower than a threshold value, that the subject has ASD or is at risk of developing ASD.

19. The method of claim 18, further comprising modulating the level of chorismate and/or the molecule within the chorismate metabolic pathway in the subject to alleviate one or more symptoms of ASD and/or of comorbidities of ASD or to reduce the risk of developing ASD.

20. The method of claim 19, wherein modulating the level of chorismate and/or the molecule within the chorismate metabolic pathway comprises administering to the subject a composition comprising chorismate, chorismic acid, and/or a salt or ester of either thereof.

21. The method of any one of claims 19-20, wherein modulating the level of chorismate and/or the molecule within the chorismate metabolic pathway comprises administering to the subject a composition comprising a chorismate prodrug.

22. The method of any one of claims 19-21, wherein modulating the level of chorismate and/or the molecule within the chorismate metabolic pathway comprises administering to the subject a composition comprising a chorismate metabolite.

23. The method of any one of claims 19-22, wherein modulating the level of chorismate and/or the molecule within the chorismate metabolic pathway comprises administering to the subject a composition that modulates expression and/or activity of one or more enzymes in the chorismate metabolic pathway.

24. The method of claim 23, wherein the composition comprises an agonist, antagonist, activator, and/or inhibitor of the one or more enzymes in the chorismate metabolic pathway.

25. The method of any one of claims 23-24, wherein the one or more enzymes comprise chorismate lyase.

26. The method of any one of claims 19-25, wherein modulating the level of chorismate and/or the molecule within the chorismate metabolic pathway comprises modulating the level of chorismate.

27. The method of any one of claims 19-26, wherein modulating the level of chorismate and/or the molecule within the chorismate metabolic pathway comprises modulating the level of the molecule within the chorismate metabolic pathway.

28. A method of treating a subject having one or more symptoms of ASD or at risk of developing ASD, the method comprising modulating a level of chorismate and/or a molecule within a chorismate metabolic pathway in the subject.

29. The method of claim 28, wherein the level is modulated in the gut of the subject.

30. The method of any one of claims 28-29, wherein modulating the level of chorismate and/or the molecule within the chorismate metabolic pathway comprises administering to the subject a composition comprising chorismate, chorismic acid, a chorismate prodrug, and/or a chorismate metabolite.

31. The method of any one of claims 28-30, wherein modulating the level of chorismate and/or the molecule within the chorismate metabolic pathway comprises administering to the subject a composition that modulates expression and/or activity of one or more enzymes in the chorismate metabolic pathway.

32. The method of claim 31, wherein the one or more enzymes comprise a chorismate branchpoint enzyme.

33. The method of claim 32, wherein the chorismate branchpoint enzyme comprises chorismate lyase.

34. The method of any one of claims 31-33, wherein the composition comprises an agonist, antagonist, activator, and/or inhibitor of the one or more enzymes in the chorismate metabolic pathway.

35. The method of any one of claims 28-34, wherein modulating the level of chorismate and/or the molecule within the chorismate metabolic pathway comprises administering to the subject a composition that modulates an amount of a microbe that produces chorismate and/or the molecule within the chorismate metabolic pathway in a gastrointestinal tract of the subject.

36. A method comprising determining an abundance of one or more microbial features in a sample obtained from a subject to predict and/or diagnose an ASD status of the subject.

37. The method of claim 36, wherein the one or more microbial features comprise a Sarcina bacterium.

38. The method of any one of claims 36-37, wherein the one or more microbial features comprise Clostridium ventriculi.

39. The method of any one of claims 36-38, further comprising determining, if the abundance of the one or more microbial features is higher than a threshold value, that the subject has or is at risk of developing ASD.

40. The method of claim 39, further comprising reducing the abundance of the one or more microbial features in the subject to alleviate one or more symptoms of ASD and/or of comorbidities of ASD or to reduce the risk of developing ASD.

41. The method of any one of claims 36-40, wherein reducing the abundance of the one or more microbial features comprises administering one or more antibiotics and/or phage therapy to the subject.

42. The method of any one of claims 36-41, further comprising determining, if the abundance of the one or more microbial features is lower than a threshold value, that the subject has or is at risk of developing ASD.

43. The method of claim 42, further comprising increasing the abundance of the one or more microbial features in the subject to alleviate one or more symptoms of ASD and/or of comorbidities of ASD or to reduce the risk of developing ASD.

44. The method of claim 43, wherein increasing the abundance of the one or more microbial features comprises administering to the subject one or more prebiotics and/or probiotics.

45. A method of assisting in the diagnosis of a subject having one or more symptoms of autism, the method comprising determining a level of proton influx in a gastrointestinal tract of the subject from a sample obtained from the subject.

46. The method of claim 45, further comprising determining, if the level of proton influx is higher than a threshold value, that the subject has or is at risk of developing ASD.

47. The method of claim 46, further comprising modulating a gastrointestinal pH of the subject to alleviate one or more symptoms of ASD and/or of comorbidities of ASD or to reduce the risk of developing ASD.

48. A method for identifying a subtype of a subject with ASD, the method comprising determining a composition of fecal microbiome or a presence of one or more microbial features in a sample obtained from the subject.

49. A method comprising determining one or more microbial features in a sample obtained from a subject and using the one or more microbial features as biomarkers for medical decision making regarding therapeutic interventions.

50. A method comprising training a machine learning model to associate microbial features with a risk or risk level of ASD and/or with candidacy or non-candidacy for a treatment for ASD in a subject.

51. A non-transitory computer-readable storage medium having encoded thereon executable instructions that, when executed by at least one processor, cause the at least one processor to perform any one or any combination of the foregoing methods.

52. An apparatus comprising at least one processor and at least one non-transitory computer-readable storage medium having encoded thereon executable instructions that, when executed by the at least one processor, cause the at least one processor to perform any one or any combination of the foregoing methods.

53. A method, comprising:
determining an abundance of one or more molecules or microbial features in a sample obtained from a subject; and
administering one or more therapies based on the absence and/or low abundance of the one or more molecules or microbial features to alleviate one or more symptoms of ASD and/or comorbidities of ASD or to reduce the risk of developing ASD.

54. A method comprising administering one or more probiotics and/or prebiotics based on the absence and/or low abundance of one or more microbial features to alleviate autistic comorbidities and phenotypes.

55. A method comprising targeting one or more microbial features based on the presence and/or excess of one or more microbial features to alleviate one or more symptoms of ASD and/or of comorbidities of ASD in a subject or to reduce the subject's risk of developing ASD.

56. The method of claim 56, wherein targeting the one or more microbial features comprises administering one or more antibiotics and/or phage therapy to the subject.

57. A method comprising targeting one or more microbial molecules associated with one or more microbial features to alleviate one or more symptoms of ASD and/or of comorbidities of ASD in a subject.

58. The method of claim 57, wherein the one or more molecules comprise one or more toxins.

59. The method of any of claims 1-58, wherein the subject has one or more signs or symptoms of ASD or is at risk of developing ASD.
